(19) United States
(12) Patent Application Publication
(10) Pub. No.: US 2025/0281056 A1
(43) Pub. Date: Sep. 11, 2025

(54) WEARABLE DEVICE WITH PHYSIOLOGICAL MONITORING

(71) Applicant: Masimo Corporation, Irvine, CA (US)

(72) Inventors: Ammar Al-Ali, San Juan Capistrano, CA (US); Stephen Scruggs, Newport Beach, CA (US); Richard Priddell, Irvine, CA (US)

(21) Appl. No.: 19/211,874

(22) Filed: May 19, 2025

Related U.S. Application Data
(63) Continuation of application No. 17/812,104, filed on Jul. 12, 2022, now Pat. No. 12,336,796.
(60) Provisional application No. 63/221,385, filed on Jul. 13, 2021.
(60) Provisional application No. 63/230,239, filed on Aug. 6, 2021.
(60) Provisional application No. 63/252,893, filed on Oct. 6, 2021.

(51) Int. Cl. A61B 5/024; A61B 5/00

(52) U.S. Cl. CPC A61B 5/02427; A61B 5/6824

(57) ABSTRACT

An optical physiological sensor can be integrated into a wearable device and can comprise a frame defining a first emitter chamber housing a first group of emitters; a second emitter chamber housing a second group of emitters; and a plurality of detector chamber housing a plurality of detectors configured to detect optical radiation emitted from the first and second groups of emitters.

### CROSS-REFERENCE TO RELATED APPLICATIONS

This application is a continuation of U.S. patent application Ser. No. 17/812,104, filed Jul. 12, 2022 which claims the benefit of U.S. Provisional Application No. 63/252,893, filed Oct. 6, 2021, and U.S. Provisional Application No. 63/230,239, filed Aug. 6, 2021, and U.S. Provisional Application No. 63/221,385, filed Jul. 13, 2021. The entire disclosure of each of the above items is hereby made part of this specification as if set forth fully herein and incorporated by reference for all purposes, for all that it contains.

### FIELD

The present disclosure relates to a wearable health monitoring device incorporating a plurality of sensors worn on the wrist.

### BACKGROUND

Spectroscopy is a common technique for measuring the concentration of organic and some inorganic constituents of a solution. The theoretical basis of this technique is the Beer-Lambert law, which states that the concentration cᵢ of an absorbent in solution can be determined by the intensity of light transmitted through the solution, knowing the pathlength d_{λ}, the intensity of the incident light I_{0,λ}, and the extinction coefficient ε_{i,λ} at a particular wavelength λ.

In generalized form, the Beer-Lambert law is expressed as:

$${I}_{λ} = {I}_{0 , λ} {e}^{- {d}_{λ} \cdot {μ}_{a , λ}} \qquad ( 1 )$$

$${μ}_{a , λ} = {\sum }_{i = 1}^{n} {ε}_{i , λ} \cdot {c}_{i} \qquad ( 2 )$$

where μ_{a,λ} is the bulk absorption coefficient and represents the probability of absorption per unit length. The minimum number of discrete wavelengths that are required to solve equations 1 and 2 is the number of significant absorbers that are present in the solution.

A practical application of this technique is pulse oximetry or plethysmography, which utilizes a noninvasive sensor to measure oxygen saturation and pulse rate, among other physiological parameters. Pulse oximetry or plethysmography relies on a sensor attached externally to the patient (typically for example, at the fingertip, foot, ear, forehead, or other measurement sites) to output signals indicative of various physiological parameters, such as a patient's blood constituents and/or analytes, including for example a percent value for arterial oxygen saturation, among other physiological parameters. The sensor has at least one emitter that transmits optical radiation of one or more wavelengths into a tissue site and at least one detector that responds to the intensity of the optical radiation (which can be reflected from or transmitted through the tissue site) after absorption by pulsatile arterial blood flowing within the tissue site. Based upon this response, a processor determines the relative concentrations of oxygenated hemoglobin (HbO₂) and deoxygenated hemoglobin (Hb) in the blood so as to derive oxygen saturation, which can provide early detection of potentially hazardous decreases in a patient's oxygen supply, and other physiological parameters.

A patient monitoring device can include a plethysmograph sensor. The plethysmograph sensor can calculate oxygen saturation (SpO₂), pulse rate, a plethysmograph waveform, perfusion index (PI), pleth variability index (PVI), methemoglobin (MetHb), carboxyhemoglobin (CoHb), total hemoglobin (tHb), respiration rate, glucose, and/or otherwise. The parameters measured by the plethysmograph sensor can display on one or more monitors the foregoing parameters individually, in groups, in trends, as combinations, or as an overall wellness or other index.

A pulse oximetry sensor is described in U.S. Pat. No. 6,088,607 entitled Low Noise Optical Probe; pulse oximetry signal processing is described in U.S. Pat. Nos. 6,650,917 and 6,699,194 entitled Signal Processing Apparatus and Signal Processing Apparatus and Method, respectively; a pulse oximeter monitor is described in U.S. Pat. No. 6,584,336 entitled Universal/Upgrading Pulse Oximeter; all of which are assigned to Masimo Corporation, Irvine, CA, and each is incorporated by reference herein in its entirety.

### SUMMARY

A drawback to current pulse oximetry sensors is a need to be located near significant capillary beds on the body, including fingers, ears, toes, nose and forehead. Such locations are often inconvenient for monitoring a user during normal activities, outside of a healthcare facility. Further, although measuring through motion oxygen saturation technology exists, it is directed to the healthcare facility context and is not reliable for normal routines, which include sporting activities or other significant daily movement. Accordingly, the present disclosure provides a sensor which allows for measuring pulse oximetry at sparse capillary bed locations, including the wrist. The present disclosure also provides algorithms for measuring pulse oximetry though higher exertion everyday motion.

A physiological monitoring sensor or module, also referred to herein as a physiological parameter measurement sensor or module, or a module, can be integrated into a wearable device that is secured to a wrist of a person (the “wearer”), such as a wristwatch or watch. The sensor on the watch can be used to monitor the wearer's physiological parameters. The sensor can detect pulse rate, oxygen saturation, hydration status, respiratory rate, and/or other parameters, such as the parameters disclosed herein, of the wearer. The sensor can include a convex protrusion to improve pressure and contact, and therefore optical coupling, between the wearer's skin and the physiological parameter measurement sensor. The curvature of the sensor can be designed to balance the desired pressure by the watch on the wearer's wrist and the wearer's comfort. The sensor can include a light barrier between emitters and detectors of the module and/or light diffusing materials surrounding the emitters and the detectors, among other features, to improve signal strength and reduce noise. The sensor or the watch can include a connection port to receive another sensor, which can be configured to be coupled to the wearer at a different measurement site of the wearer's body than the wrist. The sensor can be configured to continuously, at certain time intervals, and/or upon the wearer's request, measure one or more of the physiological parameters. For example, the sensor can be configured to continuously measure the wearer's oxygen saturation and/or pulse rate when the watch is worn on the wearer's wrist.

An example optical physiological sensor of the present disclosure can be integrated into a watch configured to monitor health of a wearer. The optical physiological sensor can be configured to face tissue of the wearer when the watch is worn by the wearer. The optical physiological sensor can be configured to measure physiological parameters of the wearer using information from the optical physiological sensor. The optical physiological sensor can comprise: a printed circuit board (PCB); a first emitter group comprising a first plurality of light emitting diodes (LEDs) at a first location on the PCB; a second emitter group comprising a second plurality of LEDs at a second location on the PCB different from the first location, wherein the second emitter group comprises the same number and type of LEDs as the first emitter group; one or more first light blocks separating the first emitter group from the second emitter group; light diffusing material configured to diffuse light emitted by each of the first and second pluralities of LEDs; a plurality of detectors including six or more photodiodes on the PCB; one or more second light blocks separating each of the detectors of the plurality of detectors from (i) each of the other detectors of the plurality of detectors and from (ii) the first and second emitter groups; and a convex surface configured to be positioned between (i) the first and second emitter groups and the six or more photodiodes and (ii) the tissue of the wearer, the convex surface comprising one or more surface materials.

In some configurations, the first plurality of LEDs may be configured to emit light of at least five wavelengths and the second plurality of LEDs may be configured to emit light of at least five wavelengths.

In some configurations, the five wavelengths emitted by the first plurality of LEDs may be the same wavelengths as the five wavelengths emitted by the second plurality of LEDs.

In some configurations, the first plurality of LEDs may comprise LEDs of a same type as LEDs of the second plurality of LEDs.

In some configurations, the LEDs of the first plurality of LEDs may be arranged on the PCB to mirror, across a centerline of the sensor, an arrangement of the LEDs of the second plurality of LEDs on the PCB.

In some configurations, the first plurality of LEDs may comprise: a first LED configured to emit light of a first wavelength, a second LED configured to emit light of a second wavelength, a third LED configured to emit light of a third wavelength, a fourth LED configured to emit light of a fourth wavelength, and a fifth LED configured to emit light of a fifth wavelength, and the second plurality of LEDs may comprise: a sixth LED configured to emit light of the first wavelength, a seventh LED configured to emit light of the second wavelength, an eighth LED configured to emit light of the third wavelength, a ninth LED configured to emit light of the fourth wavelength, and a tenth LED configured to emit light of the fifth wavelength.

In some configurations, the first LED may be located on the PCB at a location that mirrors, across a centerline of the sensor, a location of the sixth LED on the PCB; the second LED may be located on the PCB at a location that mirrors, across a centerline of the sensor, a location of the seventh LED on the PCB; the third LED may be located on the PCB at a location that mirrors, across a centerline of the sensor, a location of the eighth LED on the PCB; the fourth LED may be located on the PCB at a location that mirrors, across a centerline of the sensor, a location of the ninth LED on the PCB; the fifth LED may be located on the PCB at a location that mirrors, across a centerline of the sensor, a location of the tenth LED on the PCB; and the centerline of the sensor may bisect at least one of the plurality of detectors.

In some configurations, the centerline of the sensor may bisect two of the plurality of detectors, and each of said two detectors may be configured to generate, responsive to detecting light emitted from the first and second plurality of emitters, one or more signals for normalizing a physiological parameter measurement of the sensor.

In some configurations, the first and sixth LEDs may each be located a same distance from at least one of the plurality of detectors; the second and seventh LEDs may each be located a same distance from at least one of the plurality of detectors; the third and eighth LEDs may each be located a same distance from at least one of the plurality of detectors; the fourth and ninth LEDs may each be located a same distance from at least one of the plurality of detectors; and the fifth and tenth LEDs may each be located a same distance from at least one of the plurality of detectors.

In some configurations, the plurality of detectors may comprise: a first detector group comprising at least two detectors; and a second detector group comprising at least two detectors, and the first emitter group may be proximal to the first detector group and distal to the second detector group, and the second emitter group may be proximal to the second detector group and distal to the first detector group.

In some configurations, the first and second emitter groups may be located at a central location of the PCB of the sensor.

In some configurations, the plurality of detectors may extend around a circumference of the PCB of the sensor.

An example optical physiological sensor of the present disclosure can be integrated into a watch configured to monitor health of a wearer. The optical physiological sensor can be configured to face tissue of the wearer when the watch is worn by the wearer. The optical physiological sensor can be configured to measure physiological parameters of the wearer using information from the optical physiological sensor. The optical physiological sensor can comprise: a printed circuit board (PCB); a first emitter group comprising a first plurality of light emitting diodes (LEDs) at a first location on the PCB; a second emitter group comprising a second plurality of LEDs at a second location on the PCB different from the first location, wherein the second emitter group comprises the same number and type of LEDs as the first emitter group; one or more first light blocks separating the first emitter group from the second emitter group; light diffusing material configured to diffuse light emitted by each of the first and second pluralities of LEDs; a plurality of detectors including six or more photodiodes on the PCB; one or more second light blocks separating each of the detectors of the plurality of detectors from each of the other detectors of the plurality of detectors; and a convex surface configured to be positioned between (i) the first and second emitter groups and the six or more photodiodes and (ii) the tissue of the wearer, the convex surface comprising one or more surface materials.

In some configurations, the first emitter group can define a first group of emitters, each emitter of the first group of emitters being located on the PCB less than 1.0 mm from each of the other emitters of the first group of emitters, and wherein the second emitter group can define a second group of emitters, each emitter of the second group of emitters being located on the PCB less than 1.0 mm from each of the other emitters of the second group of emitters.

In some configurations, the plurality of detectors can comprise: a first detector group which can comprise at least two detectors, wherein each detector of the at least two detectors can be proximal to the first emitter group; a second detector group which can comprise at least two detectors, wherein each detector of the at least two detectors can be distal to the first emitter group.

In some configurations, at least two of the plurality of detectors can each individually comprise an intermediate detector for the first emitter group and the second emitter group.

In some configurations, the first and second emitter groups can be located at a central location of the PCB of the sensor.

In some configurations, the plurality of detectors can extend around a circumference of the PCB of the sensor.

In some configurations, the PCB can comprise a conductive liquid adhesive.

In some configurations, the PCB can comprise a reflective surface.

In some configurations, the first plurality of LEDs can be configured to emit light of at least four wavelengths and wherein the second plurality of LEDs can be configured to emit light of at least four wavelengths.

In some configurations, the first plurality of LEDs can comprise: a first LED which can be configured to emit light of a first wavelength, a second LED which can be configured to emit light of a second wavelength, a third LED which can be configured to emit light of a third wavelength, and a fourth LED which can be configured to emit light of a fourth wavelength, and the second plurality of LEDs can comprise: a fifth LED which can be configured to emit light of the first wavelength, a sixth LED which can be configured to emit light of the second wavelength, a seventh LED which can be configured to emit light of the third wavelength, and an eighth LED which can be configured to emit light of the fourth wavelength.

In some configurations, the sensor can comprise a sensor processor configured to drive the first plurality of LEDs and the second plurality of LEDs at varying intensities.

In some configurations, the sensor processor can be configured to drive the intensities of the first plurality of LEDs and the second plurality of LEDs based, at least in part, on one or more of an attenuation of emitted light or a temperature of the first plurality of LEDs and the second plurality of LEDs.

In some configurations, the sensor may comprise a sensor processor configured to activate or deactivate some of the detectors of the plurality of detectors.

In some configurations, the sensor may comprise an ECG sensor, and the ECG sensor may comprise a reference electrode, a negative electrode, and a positive electrode and the reference and negative electrodes can be located on the sensor and a portion of a housing of the watch can form the positive electrode.

In some configurations, the reference and negative electrodes can be releasably coupled to the PCB of the sensor.

The present disclosure provides a system for monitoring physiological parameters. The system may comprise a wearable device which may comprise one or more physiological sensors configured to monitor one or more physiological parameters of a user. The system may comprise a computing device external to the wearable device and in wireless communication with the wearable device. The computing device may be configured to: receive, via an interactive user interface, user input; in response to receiving the user input, wirelessly transmit a first signal to the wearable device to instruct the wearable device to perform a physiological monitoring operation; receive, from the wearable device, physiological parameter data measured by the physiological monitoring operation; and cause presentation of the physiological parameter data in the interactive user interface.

In some configurations, the monitoring operation can comprise continuous measuring of the physiological parameters.

In some configurations, the monitoring operation can comprise: measuring the physiological parameters for a predetermined length of time; and ceasing to measure the physiological parameters upon expiration of the predetermined length of time.

In some configurations, the wearable device can be configured to: in response to receiving the first signal from the computing device, determine a power level of a battery of the wearable device; determine whether the power level is sufficient to perform the monitoring operation; and in response to determining that the power level is sufficient, perform the monitoring operation.

In some configurations, the wearable device can be configured to: in response to receiving the first signal from the computing device, determine a power level of a battery of the wearable device; determine whether the power level is sufficient to perform the monitoring operation; in response to determining that the power level is not sufficient, determine a modified monitoring operation based at least in part on the power level; and perform the modified monitoring operation.

In some configurations, the computing device can be further configured to: cause presentation, in the interactive user interface, of one or more of a graph, chart, or trend of historical and substantially real-time physiological parameter data.

In some configurations, the wearable device can be configured to: determine that one or more physiological parameters exceed a respective predetermined threshold; and in response to determining that the one or more physiological parameters exceed the respective predetermined threshold, transmit a second signal to the computing device to cause the computing device to generate an alarm.

An example optical physiological sensor of the present disclosure can be integrated into a watch configured to monitor health of a wearer. The optical physiological sensor can be configured to face tissue of the wearer when the watch is worn by the wearer and to measure physiological parameters of the wearer using information from the optical physiological sensor. The optical physiological sensor can comprise a first emitter group comprising a first plurality of light emitting diodes (LEDs) at a first location; a second emitter group comprising a second plurality of LEDs at a second location different from the first location, wherein the second emitter group can comprise the same number and type of LEDs as the first emitter groups; one or more light blocks separating the first emitter group from the second emitter group; light diffusing material configured to diffuse light emitted by each of the first and second pluralities of LEDs; a plurality of detectors including four or more photodiodes; and a convex surface configured to be positioned between (i) the first and second emitter groups and the four or more photodiodes and (ii) the tissue of the wearer, the convex surface comprising one or more surface materials.

In some configurations, the one or more surface materials can comprise at least a portion of the one or more light blocks and a light transmission material.

In some configurations, the emitters in the first or second emitter groups may not be electrically connected to one another.

In some configurations, the first or second emitter groups can define a group of emitters located in close proximity.

In some configurations, the plurality of detectors can be individually both a near detector and far detector for each emitter group.

In some configurations, the first and second emitter groups can be located at non-central locations of a printed circuit board (PCB) of the sensor.

In some configurations, the one or more light blocks can extend from a surface of the sensor positioning the first and second pluralities of LEDs towards a tissue of the wearer when the watch is worn.

In some configurations, each of the first or second emitter group can be surrounded by its own diffusing material.

In some configurations, the light diffusing material surrounding the first emitter group can be different from the light diffusing material surrounding the second emitter group.

In some configurations, at least some of the plurality of detectors can extend around a circumference of the sensor.

In some configurations, the plurality of detectors can be positioned in a grid pattern and/or across from one another.

In some configurations, locations of the emitter groups can be interleaved with the plurality of detectors.

In some configurations, at least one of the plurality of detectors can be located between the first plurality of LEDs and the second plurality of LEDs, and at least one of the plurality of detectors can be located on each of at least two sides of each of the first plurality of LEDs and the second plurality of LEDs.

In some configurations, the sensor can further comprise a processor configured to determine an oxygen saturation measurement based on signals from the optical physiological sensor.

An example optical physiological sensor of the present disclosure can be integrated into a watch configured to monitor health of a wearer. The optical physiological sensor can be configured to face tissue of the wearer when the watch is worn by the wearer and to measure physiological parameters of the wearer using information from the optical physiological sensor. The optical physiological sensor can comprise a plurality of emitters, the emitters configured to emit light of a plurality of different wavelengths, the plurality of different wavelengths comprising at least three different wavelengths; a plurality of detectors, the detectors configured to detect light emitted by the plurality of emitters and attenuated by tissue of the user when the watch is worn on the wrist of the wearer and output signals to a sensor processor for determining the physiological parameters of the wearer; and a sensor housing, the plurality of emitters and the plurality of detectors enclosed within the housing, wherein the sensor housing can comprise: a convex skin-facing light transmissive cover extending over the plurality of emitters and the plurality of detectors, the cover located at a first side of sensor housing, and a printed circuit board (PCB) located at a second side of the sensor housing opposite the first side, the plurality of emitters and detectors located on a skin-facing side of the PCB; and a plurality of light barriers extending from the PCB to the cover, the plurality of light barriers configured to form walls of chambers to block light or substantially all the light between the chambers, each chamber enclosing one or more emitters without detectors, or one or more detectors without emitters, wherein a skin-facing surface of the cover and at least one of the light barriers can define a skin-facing surface of the sensor, a surface area of the cover extending over the chambers that enclose one or more detectors is at least 50% of a surface area of the skin-facing surface of the sensor.

In some configurations, the surface area of the cover extending over the chambers that enclose one or more detectors can be at least 100 mm².

In some configurations, the surface area of the cover extending over the chambers that enclose one or more detectors can be at least 150 mm².

In some configurations, the surface area of the cover extending over the chambers that enclose one or more detectors can be at least 165 mm².

In some configurations, a surface area of the light transmissive cover that extends over the chambers that enclose one or more emitters can be at least 25 mm².

In some configurations, the surface area of the light transmissive cover that extends over the chambers that enclose one or more detectors can be at least 35 mm².

In some configurations, the skin-facing surface of the sensor can have a longer side and a shorter side, the longer side configured to be along a width of the wearer's wrist when the watch is worn.

In some configurations, more of the plurality of detectors can be located along the longer side than along the shorter side.

In some configurations, the plurality of emitters can comprise a first group of emitters and a second group of emitters, the chambers comprising a first emitter chamber enclosing the first group and a second emitter chamber enclosing the second group.

In some configurations, the plurality of detectors can comprise a first ring of detectors and a second ring of detectors, the first ring of detectors surrounding the first group of emitters and the second ring of detectors surrounding the second group of emitters.

In some configurations, at least one of the plurality of detectors can be located between the first and second group of emitters and can be shared by the first and second rings of detectors.

In some configurations, some of the plurality of detectors can be closer to the first group of emitters than a remainder of the plurality of detectors and some of the plurality of detectors can be closer to the second group of emitters than a remainder of the plurality of detectors.

In some configurations, the plurality of light barriers can extend to a skin-facing surface of the cover.

An example optical physiological sensor of the present disclosure can be integrated into a watch configured to monitor health of a wearer. The optical physiological sensor can be configured to face tissue of the wearer when the watch is worn by the wearer and to measure physiological parameters of the wearer using information from the optical physiological sensor. The optical physiological sensor can comprise a plurality of emitters, the emitters configured to emit light of a plurality of different wavelengths, the plurality of different wavelengths comprising at least three different wavelengths; a plurality of detectors, the detectors configured to detect light emitted by the plurality of emitters and attenuated by tissue of the user when the watch is worn on the wrist of the wearer and output signals to a sensor processor for determining the physiological parameters of the wearer; and a sensor housing, the plurality of emitters and the plurality of detectors enclosed within the housing, wherein the sensor housing can comprise: a convex skin-facing light transmissive cover extending over the plurality of emitters and the plurality of detectors, the cover located at a first side of sensor housing, and a printed circuit board (PCB) located at a second side of the sensor housing opposite the first side, the plurality of emitters and detectors located on a skin-facing side of the PCB; and a plurality of light barriers extending from the PCB to the cover, the plurality of light barriers configured to form walls of chambers to block light or substantially all the light between the chambers, each chamber enclosing one or more emitters without detectors, or one or more detectors without emitters, wherein at least one of the plurality of light barriers can extend to a skin-facing surface of the cover.

In some configurations, all of the plurality of light barriers can extend to the skin-facing surface of the cover.

In some configurations, the skin-facing surface of the cover and the at least one of the light barriers can define a skin-facing surface of the sensor.

In some configurations, the skin-facing surface of the sensor can comprise a continuous curvature.

In some configurations, the cover can be a single lens or cover.

In some configurations, the cover can comprise individual lenses, each lens or cover covering a single chamber.

In some configurations, the cover can comprise a lens or cover covering all the chambers that extend over one or more detectors.

In some configurations, the lens or cover covering all the chambers that extend over one or more detectors may not cover a chamber that extends over one or more emitters.

In some configurations, the plurality of light barriers can comprise colored sapphire glass.

An example optical physiological sensor of the present disclosure can be integrated into a watch configured to monitor health of a wearer. The optical physiological sensor can be configured to face tissue of the wearer when the watch is worn by the wearer and to measure physiological parameters of the wearer using information from the optical physiological sensor. The optical physiological sensor can comprise a first emitter comprising a first a plurality of light emitting diodes (LEDs) positioned on a surface of a substrate; a first photodiode positioned on the surface of the substrate; a curved surface extending over all the first plurality of LEDs and the first photodiode; and a first light barrier positioned between the first emitter and the first photodiode, and extending from the surface of the substrate to the curved surface.

In some configurations, the first light barrier can comprise one or more portions that together extend from the surface of the substrate to the curved surface.

In some configurations, the sensor can further comprise: a second emitter comprising a second plurality of LEDs positioned on the surface of the substrate; a second photodiode positioned on the surface of the substrate; a second light barrier positioned between (i) both the first and second emitters and (ii) the second photodiode, and extending from the surface of the substrate to the curved surface, wherein the curved surface can extend over all the second plurality of LEDs and the second photodiode.

In some configurations, the second light barrier can comprise one or more portions that together extend from the surface of the substrate to the curved surface.

In some configurations, portions of the curved surface positioned above the first and second emitters can comprise at least a first material, portions of the curved surface positioned and the first and second photodiodes can comprise at least a second material, and portions of the first and second barriers extending to the curved surface can comprise at least a third material different from the first and second materials.

In some configurations, at least the first, second, and third materials together can make up the curved surface.

In some configurations, the first and second materials can comprise the same material.

An example optical physiological sensor of the present disclosure can be integrated into a watch configured to monitor health of a wearer. The optical physiological sensor can be configured to face tissue of the wearer when the watch is worn by the wearer and to measure physiological parameters of the wearer using information from the optical physiological sensor. The optical physiological sensor can comprise a plurality of light-emitting diodes (LEDs) configured to emit light to tissue of a wearer; a wall dividing the plurality of LEDs into at least a first group of LEDs and a second group of LEDs, the wall blocking at least some of the light emitted by the first group of LEDs from contacting the second group of LEDs; four or more photodiodes configured to detect the light emitted by the plurality of LEDs after attenuation by the tissue; and one or more covers covering the plurality of LEDs and the four or more photodiodes, the one or more covers together forming part of a convex surface configured to contact the tissue.

An example optical physiological sensor of the present disclosure can be integrated into a watch configured to monitor health of a wearer. The optical physiological sensor can be configured to face tissue of the wearer when the watch is worn by the wearer and to measure physiological parameters of the wearer using information from the optical physiological sensor. The optical physiological sensor can comprise a plurality of emitters, the emitters configured to emit light of a plurality of different wavelengths, the plurality of different wavelengths comprising at least three different wavelengths; a plurality of detectors, the detectors configured to detect light emitted by the plurality of emitters and attenuated by tissue of the user when the watch is worn on the wrist of the wearer and output signals to a sensor processor for determining the physiological parameters of the wearer; and a sensor housing, the plurality of emitters and the plurality of detectors enclosed within the housing, wherein the sensor housing can comprise: a convex skin-facing light transmissive cover extending over the plurality of emitters and the plurality of detectors, the cover located at a first side of sensor housing, and a printed circuit board (PCB) located at a second side of the sensor housing opposite the first side, the plurality of emitters and detectors located on a skin-facing side of the PCB; and a plurality of light barriers extending from the PCB to the cover, the plurality of light barriers configured to form walls of chambers to block light or substantially all the light between the chambers, each chamber enclosing one or more emitters without detectors, or one or more detectors without emitters, wherein the plurality of detectors can comprise a plurality of far detectors that are further from at least some of the plurality of emitters than a remainder of the plurality of detectors.

In some configurations, the plurality of emitters can comprise a first group of emitters and a second group of emitters, the chambers comprising a first emitter chamber enclosing the first group and a second emitter chamber enclosing the second group.

In some configurations, the plurality of detectors can comprise a first ring of detectors and a second ring of detectors, the first ring of detectors surrounding the first group of emitters and the second ring of detectors surrounding the second group of emitters.

In some configurations, at least one of the plurality of detectors can be located between the first and second group of emitters and is shared by the first and second rings of detectors.

In some configurations, some of the plurality of detectors can be closer to the first group of emitters than a remainder of the plurality of detectors and some of the plurality of detectors are closer to the second group of emitters than a remainder of the plurality of detectors.

In some configurations, the sensor can further comprise the sensor processor, wherein the sensor processor is configured to determine a hydration status of a user based on signals from the plurality of far detectors.

In some configurations, at least one of the emitters can be configured to emit light of a wavelength more sensitive to water than a remainder of the different wavelengths.

In some configurations, the wavelength more sensitive to water can be about 970 nm.

In some configurations, the sensor processor can be configured to compare signals of the reflected light of the wavelength more sensitive to water and another wavelength less sensitive to water from the plurality of far detectors.

In some configurations, the sensor processor can be configured to selectively drive some of the plurality of emitters and/or activate or deactivate some of the plurality of detectors.

An example optical physiological sensor of the present disclosure can be integrated into a watch configured to monitor health of a wearer. The optical physiological sensor can be configured to face tissue of the wearer when the watch is worn by the wearer and to measure physiological parameters of the wearer using information from the optical physiological sensor. The optical physiological sensor can comprise a plurality of emitters, the emitters configured to emit light of a plurality of different wavelengths, wherein at least one of the emitters can be configured to emit light of a reference wavelength; a plurality of detectors, the detectors configured to detect light emitted by the plurality of emitters and attenuated by tissue of the user when the watch is worn on the wrist of the wearer; a sensor processor, wherein the plurality of detectors can be configured to output signals to the sensor processor for determining at least some of the physiological parameters of the wearer based in part on a signal of the reflected light of the reference wavelength; and a sensor housing, the plurality of emitters and the plurality of detectors enclosed within the housing, wherein the sensor housing can comprise: a convex skin-facing light transmissive cover extending over the plurality of emitters and the plurality of detectors, the cover located at a first side of sensor housing, and a printed circuit board (PCB) located at a second side of the sensor housing opposite the first side, the plurality of emitters and detectors located on a skin-facing side of the PCB; and a plurality of light barriers extending from the printed circuit board to the cover, the plurality of light barriers configured to form walls of chambers to block light or substantially all the light between the chambers, each chamber enclosing one or more emitters without detectors, or one or more detectors without emitters.

In some configurations, the reference wavelength can be about 525 nm.

In some configurations, the light of the reference wavelength can be green or yellow.

In some configurations, the sensor processor can be configured to extract features from signals of other wavelengths based on the signal of the reflected light of the reference wavelength and calculate the at least some of the physiological parameters based on the extracted features.

In some configurations, at least one of the emitters can be configured to emit light of a wavelength more sensitive to oxygen saturation.

In some configurations, at least one of the emitters can be configured to emit light of a wavelength more sensitive to water.

In some configurations, at least one of the emitters can be configured to emit light of a normalizing wavelength.

In some configurations, the sensor processor can be configured to determine a hydration status of a user based on signals of the reflected light of the wavelength more sensitive to water and of the normalizing wavelength.

In some configurations, one or more physiological parameters can comprise a pulse rate, respiration rate, SpO2, PVI, PI, RRP, hydration, or a combination thereof.

In some configurations, the sensor can further comprise a thermistor located near the plurality of emitters.

In some configurations, the sensor can further comprise an accelerometer and/or gyroscope.

In some configurations, the sensor processor can be configured to selectively drive some of the plurality of emitters and/or activate or deactivate some of the plurality of detectors.

An example optical physiological sensor of the present disclosure can be integrated into a watch configured to monitor health of a wearer. The optical physiological sensor can be configured to face tissue of the wearer when the watch is worn by the wearer and to measure physiological parameters of the wearer using information from the optical physiological sensor. The optical physiological sensor can comprise a plurality of emitters, the emitters configured to emit light of a plurality of different wavelengths, the plurality of different wavelengths comprising at least three different wavelengths; a plurality of detectors, the detectors configured to detect light emitted by the plurality of emitters and attenuated by tissue of the user when the watch is worn on the wrist of the wearer and output signals to a sensor processor for determining the physiological parameters of the wearer; and a sensor housing, the plurality of emitters and the plurality of detectors enclosed within the housing, wherein the sensor housing can comprise: a convex skin-facing light transmissive cover extending over the plurality of emitters and the plurality of detectors, the cover located at a first side of sensor housing, and a printed circuit board (PCB) located at a second side of the sensor housing opposite the first side, the plurality of emitters and detectors located on a skin-facing side of the PCB; a plurality of light barriers extending from the PCB to the cover, the plurality of light barriers configured to form walls of chambers to block light or substantially all the light between the chambers, each chamber enclosing one or more emitters without detectors, or one or more detectors without emitters, wherein each chamber that encloses one or more emitters can be filled with a diffusing material such that there is no air gap between the plurality of emitters and the cover.

In some configurations, the light diffusing material can comprise glass microspheres.

In some configurations, the cover can comprise glass microspheres.

In some configurations, the sensor housing can comprise one or more openings configured to receive a flow of light diffusing solution.

In some configurations, the light diffusion solution can be UV-cured after being injected into each chamber that encloses one or more emitters.

In some configurations, the sensor housing can comprise one or more air vent openings configured to receive air displaced from the chamber(s) by the flow of light diffusing solution.

In some configurations, each chamber that encloses one or more detectors can be filled with the diffusing material such that there is no air gap between the plurality of detectors and the cover.

In some configurations, the diffusing material in each chamber that encloses one or more emitters can be configured to improve mixing of light such that light emitted by one of the emitter in the same chamber appears to be emitted from the entire same chamber.

An example watch of the present disclosure can be configured to monitor physiological parameters of a wearer. The watch can comprise any of the optical sensor or physiological parameter measurement sensor configurations disclosed above; a watch processor separate from and in electrical communication with the sensor processor; a power source configured to power the watch and the sensor, and a display in communication with the processor, the display configured to display the plurality of physiological parameters monitored by the sensor.

In some configurations, the display can be configured to display the wearer's SpO2 and pulse rate that are monitored by the sensor.

In some configurations, the sensor can be configured to continuously monitor the wearer's SpO2 and pulse rate.

In some configurations, the display can be configured to continuously display the wearer's SpO2 and pulse rate.

In some configurations, the watch can further comprise an ECG sensor.

In some configurations, the ECG sensor can comprise a reference electrode, a negative electrode, and a positive electrode.

In some configurations, the reference and negative electrodes can be located on the sensor.

In some configurations, a portion of a housing of the watch can form the positive electrode.

In some configurations, the ECG sensor can be in electrical communication with the sensor processor.

In some configurations, the watch can further comprise a wireless transmitter such that the watch is configured to wireless connect to external devices and/or external sensors.

In some configurations, the wireless transmitter can be a Bluetooth chip.

In some configurations, the external devices and/or external sensors can comprise a bedside monitor, a mobile communication device, a tablet, a nurses' station system, or a different medical device.

A health monitoring watch of the present disclosure can comprise a strap and a housing. The housing can comprise: a first chamber comprising a first well comprising a first depth below a first surface configured to be in contact with a skin of a user; a first plurality of light emitting diodes positioned at the first depth inside the first well, said first plurality of light emitting diodes comprising a first light emitting diode configured to emit light at a first wavelength, a second light emitting diode configured to emit light at a second wavelength different than the first wavelength, and a third light emitting diode configured to emit light at a third wavelength different than the first wavelength and the second wavelength, and a first wall surrounding the first well; a second chamber comprising a second well comprising a second depth below a second surface configured to be in contact with the skin of the user, a second plurality of light emitting diodes positioned at the second depth inside the second well, said second plurality of light emitting diodes comprising a fourth light emitting diode configured to emit light at the first wavelength, a fifth light emitting diode configured to emit light at the second wavelength different than the first wavelength, and a sixth light emitting diode configured to emit light at the third wavelength different than the first wavelength and the second wavelength, and a second wall surrounding the second well; and four or more light sensors.

A wearable health monitoring device can be configured to be worn on a wrist of a user and monitor one or more physiological parameters indicative of the user's health. The wearable health monitoring device can comprise: a first emitter group, the first emitter group comprising a first plurality of light-emitting diodes (LEDs) configured to emit light of one or more wavelengths, wherein the first emitter group can be arranged at a first location, the first location being spaced from an axis extending through a center of the wearable health monitoring device; a second emitter group, the second emitter group comprising a second plurality of LEDs configured to emit light of one or more wavelengths, wherein the second emitter group can be arranged at a second location, the second location being spaced from the first location and spaced from the axis extending through the center of the wearable health monitoring device; one or more light blocks separating the first emitter group from the second emitter group; a first light diffusing material, the first light diffusing material configured to be positioned between the first emitter group and tissue of the user when the wearable health monitoring device is in use, wherein the first light diffusing material can be configured to spread light emitted from one or more of the first plurality of LEDs before the emitted light reaches the tissue; a second light diffusing material, the second light diffusing material configured to be positioned between the second emitter group and the tissue of the user when the wearable health monitoring device is in use, wherein the second light diffusing material can be configured to spread light emitted from one or more of the second plurality of LEDs before the emitted light reaches the tissue; a plurality of photodiodes configured to detect at least a portion of the light emitted from one or more of the first plurality of LEDs or one or more of the second plurality of LEDs after attenuation through the user's tissue, the plurality of photodiodes configured to output one or more signals responsive to the detected light; and a processor configured to receive and process one or more signals responsive to the one or more signals outputted by the plurality of photodiodes and further configured to determine a physiological parameter of the user based on the received and processed one or more signals.

The present disclosure provides an optical physiological sensor integrated into a wearable device. The optical physiological sensor can comprise: a substrate having an optical center; a first emitter group of light emitting diodes (LEDs) positioned adjacent to the optical center of the substrate and spaced at an offset from the optical center; a second emitter group of LEDs positioned adjacent to the optical center of the substrate at an offset to the optical center and spaced at an offset from the optical center opposite the first emitter group of LEDs relative to the optical center; and a plurality of detectors arranged in a spatial configuration that surrounds the first and the second emitter group. Each of the plurality of detectors can be positioned on the substrate a same distance away from the optical center of the substrate.

In some configurations, the spatial configuration includes an annulus.

In some configurations, the annulus has a same optical center as the substrate.

In some configurations, the annulus is concentric with a perimeter of the sensor.

In some configurations, the annulus includes a radius of between about 6.2 mm and about 6.6 mm.

In some configurations, the annulus includes a radius of between about 6.15 mm and about 6.45 mm.

In some configurations, the sensor includes a radius of between about 15.0 mm and about 15.5 mm.

In some configurations, a ratio of a radius of the annulus to a radius of the sensor is between about 40% and about 45%.

In some configurations, the second emitter group is spaced less than 1.5 mm away from the first emitter group.

In some configurations, the second emitter group is spaced about 1.28 mm away from the first emitter group.

In some configurations, the second emitter group is spaced about 1.2 mm away from the first emitter group.

In some configurations, the sensor can further comprise a light barrier construct mounted on the substrate and configured to isolate the first emitter group, the second emitter group, and the plurality of detectors.

In some configurations, the light barrier construct is a single integrated unit comprising a plurality of light barriers defining one or more chambers.

In some configurations, the light barrier construct comprises a plurality of light barriers configured to isolate the first and second emitter groups from each of the plurality of detectors.

In some configurations, the light barrier construct comprises a light barrier configured to isolate the first emitter group from the second emitter group.

In some configurations, the light barrier includes a width of between about 1.25 mm and about 1.35 mm separating the first and second emitter groups.

In some configurations, the light barrier includes a width of between about 1.1 mm and about 1.3 mm separating the first and second emitter groups.

In some configurations, the light barrier includes a width of less than a distance from an emitter chamber defined by the light barrier construct and housing the first emitter group to a detector chamber defined by the light barrier construct and housing a detector bisected by a centerline of the sensor.

In some configurations, the light barrier construct includes a height of between about 2.80 mm and about 2.90 mm extending away from the optical center of the substrate.

In some configurations, the light barrier construct includes a height of between about 2.55 mm and about 2.65 mm extending away from the optical center of the substrate.

In some configurations, the light barrier construct includes a height of between about 1.25 mm and about 1.35 mm extending away from a perimeter of the substrate.

In some configurations, the light barrier construct includes a height of between about 1.75 mm and about 1.85 mm extending away from a perimeter of the substrate.

In some configurations, a ratio of a maximum height of the light barrier construct extending away from the substrate to a minimum height of the light barrier construct extending away from the substrate is between about 215% and about 225%.

In some configurations, a ratio of a maximum height of the light barrier construct extending away from the substrate to a minimum height of the light barrier construct extending away from the substrate is between about 145% and about 155%.

In some configurations, the light barrier construct includes a maximum height extending away from the substrate at the optical center of the substrate.

In some configurations, the light barrier defines a plurality of chambers, including, at least, a detector chamber housing a detector bisected by a centerline of the sensor; a first emitter chamber housing the first emitter group; and a second emitter chamber housing the second emitter group.

In some configurations, a distance from the first emitter chamber to the detector chamber extending along a length parallel to the centerline of the sensor is the same as a distance from the second emitter chamber to the detector chamber extending along a length parallel to the centerline of the sensor.

In some configurations, a distance from the first emitter chamber to the detector chamber extending along a length parallel to the centerline of the sensor is less than half a width of the first emitter chamber along a length parallel to the centerline of the sensor.

In some configurations, a distance from the first emitter chamber to the detector chamber extending along a length parallel to the centerline of the sensor is greater than half a width of the first emitter chamber along a length parallel to the centerline of the sensor.

In some configurations, the first emitter group includes two or more LEDs and wherein the second emitter group includes two or more LEDs.

In some configurations, the first emitter group includes three or more LEDs and wherein the second emitter group includes three or more LEDs.

In some configurations, the first emitter group includes four or more LEDs and wherein the second emitter group includes four or more LEDs.

In some configurations, the first emitter group includes five LEDs and wherein the second emitter group includes five LEDs.

In some configurations, each LED in the first emitter group emits light at a different wavelength than each of the other LEDs in the first emitter group.

In some configurations, each LED in the second emitter group emits light at a different wavelength that each of the other LEDs in the second emitter group.

In some configurations, the first emitter group is configured to emit a same plurality of light wavelengths as the second emitter group.

In some configurations, an arrangement of the LEDs of the first emitter group on the substrate mirrors an arrangement of the LEDs of the second emitter group on the substrate across a centerline of the sensor that bisects the sensor.

In some configurations, the optical center of the substrate is located on the centerline of the sensor.

In some configurations, the centerline of the sensor bisects at least one of the plurality of detectors.

In some configurations, a distance from the first emitter group to the at least one of the plurality of detectors is substantially similar to a distance from the second emitter group to the at least one of the plurality of detectors.

In some configurations, the sensor further comprises an ECG sensor, and the ECG sensor comprises a reference electrode, a negative electrode, and a positive electrode and the reference and negative electrodes are located on the sensor and the positive electrode is located on a housing of the wearable device.

In some configurations, the reference and negative electrodes are releasably coupled to the substrate of the sensor via one or more springs configured to bias the reference and negative electrodes away from the substrate.

In some configurations, the reference electrode extends along a circumference of the sensor on a first side of the sensor, and wherein the negative electrode extends along a circumference of the sensor on a second side of the sensor.

In some configurations, the reference electrode is substantially semi-annular and wherein the negative electrode is substantially semi-annular.

In some configurations, the reference and negative electrodes surround the plurality of detectors.

In some configurations, the substrate comprises a conductive liquid adhesive configured to facilitate a conductive electrical connection between the electrodes of the ECG sensor and the substrate.

In some configurations, the substrate comprises a printed circuit board (PCB).

In some configurations, the substrate comprises a reflective surface.

In some configurations, the sensor further comprises a first temperature sensor positioned on the substrate adjacent to the first emitter group of LEDs, and a second temperature sensor adjacent to the second emitter group of LEDs.

In some configurations, the first emitter group of LEDs is configured to emit light of at least four wavelengths and wherein the second emitter group of LEDs is configured to emit light of at least four wavelengths.

In some configurations, the four wavelengths emitted by the first emitter group of LEDs are the same wavelengths as the four wavelengths emitted by the second emitter group of LEDs.

In some configurations, the first emitter group of LEDs comprise LEDs of a same type as the second emitter group of LEDs.

In some configurations, the first emitter group of LEDs comprises: a first LED configured to emit light of a first wavelength, a second LED configured to emit light of a second wavelength, a third LED configured to emit light of a third wavelength, and a fourth LED configured to emit light of a fourth wavelength, and the second emitter group of LEDs comprises: a fifth LED configured to emit light of the first wavelength, a sixth LED configured to emit light of the second wavelength, a seventh LED configured to emit light of the third wavelength, and an eighth LED configured to emit light of the fourth wavelength.

In some configurations, the first LED is located on the PCB at a location that mirrors, across a centerline of the sensor, a location of the fifth LED on the PCB; the second LED is located on the PCB at a location that mirrors, across the centerline of the sensor, a location of the sixth LED on the PCB; the third LED is located on the PCB at a location that mirrors, across the centerline of the sensor, a location of the seventh LED on the PCB; and the fourth LED is located on the PCB at a location that mirrors, across the centerline of the sensor, a location of the eighth LED on the PCB.

In some configurations, the first and fifth LEDs are each located a same distance from at least one of the plurality of detectors; the second and sixth LEDs are each located a same distance from at least one of the plurality of detectors; the third and seventh LEDs are each located a same distance from at least one of the plurality of detectors; and the fourth and eighth LEDs are each located a same distance from at least one of the plurality of detectors.

In some configurations, light emitted from the first emitter group of LEDs travels along a light path to a first detector of the plurality of detectors that is substantially similar in length to a light path along which light emitted from the second emitter group of LEDs travels to the first detector.

In some configurations, the plurality of detectors includes a first detector group and a second detector group.

In some configurations, the first detector group includes at least two detectors housed in respective detector chambers, and wherein the second detector group includes at least two detectors housed in respective detector chambers.

In some configurations, a distance between the first emitter group and the first detector group is greater than a distance between the second emitter group and the first detector group.

In some configurations, a distance between the first emitter group and a first detector of the first detector group is substantially similar as a distance between the first emitter group and a second detector of the first detector group.

In some configurations, a distance between the first emitter group and the first detector group is substantially similar to a distance between the second emitter group and the second detector group.

In some configurations, light emitted from the first emitter group travels to the first detector group along a light path that is shorter than a light path along which light emitted from the second emitter group travels to the first detector group.

In some configurations, light emitted from the second emitter group and detected by first detector group penetrates deeper into a tissue of the wearer than light emitted from the first emitter group and detected by first detector group.

In some configurations, the sensor further comprises a light barrier construct mounted on the substrate and configured to isolate the first emitter group, the second emitter group, and the plurality of detectors, wherein the light barrier construct comprises a plurality of light barriers defining one or more chambers.

In some configurations, the one or more chambers includes a first emitter chamber configured to house the first emitter group of light emitting diodes (LEDs).

In some configurations, the one or more chambers includes a second emitter chamber configured to house the second emitter group of light emitting diodes (LEDs).

In some configurations, the one or more chambers includes a plurality of detector chambers configured to house the plurality of detectors.

In some configurations, the light barrier construct is further configured to prevent the transmission of light therethrough.

In some configurations, the sensor further comprises a convex surface configured to be positioned between the tissue of the wearer and the emitter and detector chambers, the convex surface comprising at least a portion of the light barrier construct.

In some configurations, the convex surface further includes one or more lenses defining a surface of the emitter and detector chambers, wherein the one more lenses are further configured to transmit light.

In some configurations, the one or more lenses include polycarbonate and are configured to diffuse light.

In some configurations, the emitter and detector chambers enclose light diffusing material or encapsulant.

In some configurations, the light diffusing material or encapsulant includes glass beads or microspheres.

In some configurations, a height of the convex surface from the optical center of the substrate is between about 2.80 mm and about 2.90 mm.

In some configurations, a height of the convex surface from the optical center of the substrate is between about 2.55 mm and about 2.65 mm.

In some configurations, a centerline of the sensor bisects a first detector of the plurality of detectors, and wherein said first detector is configured to generate, responsive to detecting light emitted from the first and second emitter groups, one or more signals for calibrating or normalizing a physiological parameter measurement of the sensor.

In some configurations, the sensor processor is further configured to calibrate or normalize the physiological parameter measurement of the sensor based at least in part on comparing a first signal generated by the first detector responsive to light emitted from the first emitter group with a second signal generated by the first detector responsive to light emitted from the second emitter group.

In some configurations, the sensor further comprises a sensor processor configured to drive the LEDs of the first and second emitter groups at varying intensities.

In some configurations, the sensor processor is configured to drive the intensities of the LEDs of the first and second emitter groups based, at least in part, on one or more of an attenuation of emitted light or a temperature of the LEDs of the first and second emitter groups.

In some configurations, the sensor further comprises a sensor processor configured to activate or deactivate some of the detectors of the plurality of detectors.

The present disclosure provides an optical physiological sensor integrated into a wearable device. The optical physiological sensor can comprise: a first emitter group of light emitting diodes (LEDs) mounted on a substrate; a second emitter group of LEDs mounted on the substrate and spaced away from the first emitter group of LEDs, wherein the first emitter group is isolated from the second emitter group; and at least six detectors mounted on the substrate and arranged in a spatial configuration that surrounds the first and the second emitter group and isolated from each other, the first emitter group, and the second emitter group.

In some configurations, the spatial configuration includes an annulus.

In some configurations, the annulus has a same geometric center as the substrate or as the sensor.

In some configurations, the first and second emitter groups are located at a central location of the substrate.

In some configurations, the first and second emitter groups are located adjacent to a geometric center of the substrate or a geometric center of the sensor.

In some configurations, the sensor further comprises a light barrier construct mounted on the substrate and configured to isolate the first emitter group, the second emitter group, and the at least six detectors.

In some configurations, the sensor further comprises an ECG sensor, the ECG sensor comprises a reference electrode, a negative electrode, and a positive electrode and the reference and negative electrodes are located on the sensor and the positive electrode is located on a housing of the wearable device.

In some configurations, the reference and negative electrodes are releasably coupled to the substrate of the sensor via one or more springs configured to bias the reference and negative electrodes away from the substrate.

In some configurations, the reference electrode extends along a circumference of the sensor on a first side of the sensor, and wherein the negative electrode extends along a circumference of the sensor on a second side of the sensor.

In some configurations, the reference electrode is substantially semi-annular and the negative electrode is substantially semi-annular.

In some configurations, the reference and negative electrodes surround the plurality of detectors.

In some configurations, the substrate comprises a conductive liquid adhesive configured to facilitate a conductive electrical connection between the electrodes of the ECG sensor and the substrate.

In some configurations, the substrate comprises a printed circuit board (PCB).

In some configurations, the substrate comprises a reflective surface.

In some configurations, the first emitter group includes five LEDs and wherein the second emitter group includes five LEDs.

In some configurations, the sensor further comprises a first temperature sensor positioned on the substrate adjacent to the first emitter group of LEDs, and a second temperature sensor adjacent to the second emitter group of LEDs.

In some configurations, a centerline of the sensor bisects a first detector of the plurality of detectors, and said first detector is configured to generate, responsive to detecting light emitted from the first and second emitter groups, one or more signals for calibrating or normalizing a physiological parameter measurement of the sensor.

In some configurations, the sensor processor is further configured to calibrate or normalize the physiological parameter measurement of the sensor based at least in part on comparing a first signal generated by the first detector responsive to light emitted from the first emitter group with a second signal generated by the first detector responsive to light emitted from the second emitter group.

In some configurations, the sensor further comprises a sensor processor configured to drive the LEDs of the first and second emitter groups at varying intensities.

In some configurations, the sensor processor is configured to drive the intensities of the LEDs of the first and second emitter groups based, at least in part, on one or more of an attenuation of emitted light or a temperature of the LEDs of the first and second emitter groups.

In some configurations, the sensor further comprises a sensor processor configured to activate or deactivate some of the detectors of the plurality of detectors.

The present disclosure provides an optical physiological sensor integrated into a wearable device. The optical physiological sensor can comprise: a first emitter group of light emitting diodes (LEDs) mounted on a substrate; a second emitter group of LEDs mounted on the substrate and spaced away from the first emitter group of LEDs; and a plurality of detectors mounted on the substrate and arranged in an annular configuration that surrounds the first and the second emitter group, the detectors can be substantially rectangular having a first side of a length and a second side of a width, wherein the detectors are oriented on the substrate such that the first side of each detector having the length is substantially orthogonal to a radius of the annular configuration.

The present disclosure provides an optical physiological sensor integrated into a wearable device. The optical physiological sensor can comprise: a first emitter group of light emitting diodes (LEDs) mounted on a substrate; a second emitter group of LEDs mounted on the substrate and spaced away from the first emitter group of LEDs, wherein the LEDs of the first emitter group are arranged on the substrate to mirror, across a centerline of the sensor, an arrangement of the LEDs of the second emitter group on the substrate; and a plurality of detectors mounted on the substrate and arranged in a spatial configuration that surrounds the first and the second emitter group.

In some configurations, the centerline of the sensor bisects at least one of the plurality of detectors.

In some configurations, the first emitter group of LEDs is configured to emit light of at least four wavelengths and wherein the second emitter group of LEDs is configured to emit light of at least four wavelengths.

In some configurations, the four wavelengths emitted by the first emitter group of LEDs are the same wavelengths as the four wavelengths emitted by the second emitter group of LEDs.

In some configurations, the first emitter group of LEDs comprise LEDs of a same type as the second emitter group of LEDs.

In some configurations, the first emitter group of LEDs comprise: a first LED configured to emit light of a first wavelength, a second LED configured to emit light of a second wavelength, a third LED configured to emit light of a third wavelength, and a fourth LED configured to emit light of a fourth wavelength, and the second emitter group of LEDs comprises: a fifth LED configured to emit light of the first wavelength, a sixth LED configured to emit light of the second wavelength, a seventh LED configured to emit light of the third wavelength, and an eighth LED configured to emit light of the fourth wavelength.

In some configurations, the first LED is located on the PCB at a location that mirrors, across a centerline of the sensor, a location of the fifth LED on the PCB; and the second LED is located on the PCB at a location that mirrors, across the centerline of the sensor, a location of the sixth LED on the PCB; the third LED is located on the PCB at a location that mirrors, across the centerline of the sensor, a location of the seventh LED on the PCB; and the fourth LED is located on the PCB at a location that mirrors, across the centerline of the sensor, a location of the eighth LED on the PCB.

In some configurations, the first and fifth LEDs are each located a same distance from at least one of the plurality of detectors; the second and sixth LEDs are each located a same distance from at least one of the plurality of detectors; the third and seventh LEDs are each located a same distance from at least one of the plurality of detectors; and the fourth and eighth LEDs are each located a same distance from at least one of the plurality of detectors.

In some configurations, light emitted from the first emitter group of LEDs travels along a light path to a first detector of the plurality of detectors that is substantially similar in length to a light path along which light emitted from the second emitter group of LEDs travels to the first detector.

In some configurations, a centerline of the sensor bisects a first detector of the plurality of detectors, and wherein said first detector is configured to generate, responsive to detecting light emitted from the first and second emitter groups, one or more signals for calibrating or normalizing a physiological parameter measurement of the sensor.

In some configurations, the sensor processor is further configured to calibrate or normalize the physiological parameter measurement of the sensor based at least in part on comparing a first signal generated by the first detector responsive to light emitted from the first emitter group with a second signal generated by the first detector responsive to light emitted from the second emitter group.

In some configurations, the sensor further comprises a sensor processor configured to drive the LEDs of the first and second emitter groups at varying intensities.

In some configurations, the sensor processor is configured to drive the intensities of the LEDs of the first and second emitter groups based, at least in part, on one or more of an attenuation of emitted light or a temperature of the LEDs of the first and second emitter groups.

In some configurations, the sensor further comprises a sensor processor configured to activate or deactivate some of the detectors of the plurality of detectors.

The present disclosure provides an optical physiological sensor integrated into a wearable device. The optical physiological sensor can comprise: a first emitter group of light emitting diodes (LEDs) mounted on a substrate; a second emitter group of LEDs mounted on the substrate and spaced away from the first emitter group of LEDs, wherein the second emitter group is isolated from the first emitter group; and a plurality of detectors mounted on the substrate, wherein the plurality of detectors is arranged in a spatial configuration that surrounds the first and the second emitter group and isolated from each other, wherein the plurality of detectors includes a first detector group and a second detector group, and the first emitter group can be proximal to the first detector group and distal to the second detector group, and the second emitter group can be proximal to the second detector group and distal to the first detector group.

In some configurations, the first detector group includes at least two detectors housed in respective detector chambers, and wherein the second detector group includes at least two detectors housed in respective detector chambers.

In some configurations, a distance between the first emitter group and the first detector group is greater than a distance between the second emitter group and the first detector group.

In some configurations, a distance between the first emitter group and a first detector of the first detector group is substantially similar as a distance between the first emitter group and a second detector of the first detector group.

In some configurations, a distance between the first emitter group and the first detector group is substantially similar to a distance between the second emitter group and the second detector group.

In some configurations, light emitted from the first emitter group travels to the first detector group along a light path that is shorter than a light path along which light emitted from the second emitter group travels to the first detector group.

In some configurations, light emitted from the second emitter group and detected by first detector group penetrates deeper into a tissue of the wearer than light emitted from the first emitter group and detected by first detector group.

In some configurations, a centerline of the sensor bisects a first detector of the plurality of detectors, and wherein said first detector is configured to generate, responsive to detecting light emitted from the first and second emitter groups, one or more signals for calibrating or normalizing a physiological parameter measurement of the sensor.

In some configurations, the sensor processor is further configured to calibrate or normalize the physiological parameter measurement of the sensor based at least in part on comparing a first signal generated by the first detector responsive to light emitted from the first emitter group with a second signal generated by the first detector responsive to light emitted from the second emitter group.

In some configurations, the sensor further comprises a sensor processor configured to drive the LEDs of the first and second emitter groups at varying intensities.

In some configurations, the sensor processor is configured to drive the intensities of the LEDs of the first and second emitter groups based, at least in part, on one or more of an attenuation of emitted light or a temperature of the LEDs of the first and second emitter groups.

In some configurations, the sensor further comprises a sensor processor configured to activate or deactivate some of the detectors of the plurality of detectors.

The present disclosure provides an optical physiological sensor integrated into a wearable device. The optical physiological sensor can comprise: first and second emitter groups of light emitting diodes mounted on a substrate; a first emitter chamber housing the first emitter group of light emitting diodes (LEDs); a second emitter chamber housing the second emitter group of LEDs wherein the first emitter group is isolated from the second emitter group; a plurality of detector chambers housing detectors mounted on the substrate and arranged in a spatial configuration that surrounds the first and the second emitter chambers; a light barrier construct mounted on the substrate and including one or more light barriers configured to prevent the transmission of light therethrough, wherein the light barriers of the light barrier construct isolate the emitter chambers and the detector chambers; and a convex surface configured to be positioned between the tissue of the wearer and the emitter and detector chambers, the convex surface comprising at least a portion of the light barrier construct.

In some configurations, the convex surface further includes one or more lenses defining a surface of the emitter and detector chambers, wherein the one more lenses are further configured to transmit light.

In some configurations, the one or more lenses include polycarbonate and are configured to diffuse light.

In some configurations, the emitter and detector chambers enclose light diffusing material or encapsulant.

In some configurations, the light diffusing material or encapsulant includes glass beads or microspheres.

In some configurations, a height of the convex surface from a center of the substrate is between about 2.80 mm and about 2.90 mm.

In some configurations, a height of the convex surface from a center of the substrate is between about 2.55 mm and about 2.65 mm.

In some configurations, a centerline of the sensor bisects a first detector of the detectors, and wherein said first detector is configured to generate, responsive to detecting light emitted from the first and second emitter groups, one or more signals for calibrating or normalizing a physiological parameter measurement of the sensor.

In some configurations, the sensor processor is further configured to calibrate or normalize the physiological parameter measurement of the sensor based at least in part on comparing a first signal generated by the first detector responsive to light emitted from the first emitter group with a second signal generated by the first detector responsive to light emitted from the second emitter group.

In some configurations, the sensor further comprises a sensor processor configured to drive the LEDs of the first and second emitter groups at varying intensities.

In some configurations, the sensor processor is configured to drive the intensities of the LEDs of the first and second emitter groups based, at least in part, on one or more of an attenuation of emitted light or a temperature of the LEDs of the first and second emitter groups.

In some configurations, the sensor further comprises a sensor processor configured to activate or deactivate some of the detectors of the plurality of detectors.

The present disclosure provides an optical physiological sensor integrated into a wearable device. The optical physiological sensor can comprise: a circuit layer; a light barrier construct positioned proximate said circuit layer, the light barrier construct comprising a plurality of openings defining at least a first emitter chamber, a second emitter chamber, and a plurality of detector chambers. The first and second emitter chambers can be spaced from one another and are spaced inward from and surrounded by the plurality of detector chambers; and the first emitter chamber, the second emitter chamber, and the plurality of detector chambers can be isolated from one another by portions of the light barrier construct. In some configurations, the sensor comprises: a first plurality of emitters mounted on said circuit layer and positioned within the first emitter chamber of the light barrier construct; a second plurality of emitters mounted on said circuit layer and positioned within the second emitter chamber of the light barrier construct; and a plurality of detectors, each of the plurality of detectors mounted on said circuit layer and positioned within a different one of the plurality of detector chambers of the light barrier construct. In some configurations, each of the plurality of detector chambers includes only one detector.

In some configurations, the sensor further comprises a first electrocardiogram (ECG) electrode and a second ECG electrode, and wherein the first and second ECG electrodes partially surround the first plurality of emitters, second plurality of emitters, and plurality of detectors.

In some configurations, each of the first and second ECG electrodes comprises a semi-annular shape having a first end and a second end; and the first ends of the first and second ECG electrodes are separated from one another by a first gap and the second ends of the first and second ECG electrodes are separated from one another by a second gap.

In some configurations, the light barrier construct further comprises: a first recess sized and shaped to receive the first ECG electrode; and a second recess sized and shaped to receive the second ECG electrode; each of the first and second recesses comprises a semi-annular shape having a first end and a second end; the first ends of the first and second recesses are separated from one another by said first gap and the second ends of the first and second recesses are separated from one another by said second gap; and the first and second recesses partially surround the first emitter chamber, second emitter chamber, and plurality of detector chambers.

In some configurations, the plurality of detector chambers are spaced inward from the first and second recesses.

In some configurations, the sensor does not include any emitters positioned outward from the first and second ECG electrodes.

In some configurations, the sensor does not include any detectors positioned outward from the first and second ECG electrodes.

In some configurations, the first and second gaps are substantially equal.

In some configurations, first and second ends of the first and second ECG electrodes comprise a rounded shape.

In some configurations, each of the first and second gaps are between approximately 1.5 mm and approximately 1.75 mm.

In some configurations, each of the first and second gaps are between approximately 0.5 mm and approximately 0.75 mm.

In some configurations, the sensor does not include any emitters positioned within any of the plurality of detector chambers.

In some configurations, the sensor does not include any detectors positioned within any of the first and second emitter chambers.

In some configurations, the plurality of detector chambers are arranged in a circular configuration around said first and second emitter chambers.

In some configurations, said sensor does not include any emitters other than the first and second plurality of emitters.

In some configurations, said sensor does not include any detectors other than the plurality of detectors.

In some configurations, said circuit layer comprises a printed circuit board.

In some configurations, each of the first and second plurality of emitters comprises a light emitting diode (LED).

In some configurations, each of the plurality of detectors comprises a photodiode.

In some configurations, each of the first and second plurality of emitters are configured to emit light into tissue of a user; each of the plurality of detectors are configured to: (i) detect at least a portion of light emitted by at least one of the first and second plurality of emitters after attenuation by said tissue; and (ii) output at least one signal responsive to detected light; and the sensor further comprises one or more processors configured to receive and process the outputted at least one signal and determine at least one physiological parameter of the user.

The present disclosure provides a system for monitoring physiological parameters. The system can comprise: a wearable device comprising one or more physiological sensors configured to monitor one or more physiological parameters of a user; and a computing device external to the wearable device and in wireless communication with the wearable device, the computing device configured to: receive, via an interactive user interface, user input; in response to receiving the user input, wirelessly transmit a first signal to the wearable device to instruct the wearable device to perform a physiological monitoring operation; receive, from the wearable device, physiological parameter data measured by the physiological monitoring operation; and cause presentation of the physiological parameter data in the interactive user interface.

In some configurations, the monitoring operation comprises continuous measuring of the physiological parameters.

In some configurations, the monitoring operation comprises: measuring the physiological parameters for a predetermined length of time; and ceasing to measure the physiological parameters upon expiration of the predetermined length of time.

In some configurations, the wearable device is configured to: in response to receiving the first signal from the computing device, determine a power level of a battery of the wearable device; determine whether the power level is sufficient to perform the monitoring operation; and in response to determining that the power level is sufficient, perform the monitoring operation.

In some configurations, the wearable device is configured to: in response to receiving the first signal from the computing device, determine a power level of a battery of the wearable device; determine whether the power level is sufficient to perform the monitoring operation; in response to determining that the power level is not sufficient, determine a modified monitoring operation based at least in part on the power level; and perform the modified monitoring operation.

In some configurations, the wearable device is configured to: determine that one or more physiological parameters exceed a respective predetermined threshold; and in response to determining that the one or more physiological parameters exceed the respective predetermined threshold, transmit a second signal to the computing device to cause the computing device to generate an alarm.

In some configurations, the computing device is further configured to: cause presentation, in the interactive user interface, of one or more of a graph, chart, or trend of historical and substantially real-time physiological parameter data.

It is noted that “plethysmograph” as used herein (commonly referred to as “photoplethysmograph”), encompasses its broad ordinary meaning known to one of skill in the art, which includes at least data representative of a change in the absorption of particular wavelengths of light as a function of the changes in body tissue resulting from pulsing blood. Moreover, “oximetry” as used herein encompasses its broad ordinary meaning known to one of skill in the art, which includes at least those noninvasive procedures for measuring parameters of circulating blood through spectroscopy.

For purposes of summarization, certain aspects, advantages and novel features are described herein. Of course, it is to be understood that not necessarily all such aspects, advantages or features need to be present in any particular aspect.

### BRIEF DESCRIPTION OF THE DRAWINGS

The drawings and the associated descriptions are provided to illustrate aspects of the disclosure and not to limit the scope of the claims. In the present disclosure, “bottom” refers to the side facing a wearer's wrist when an example wearable device disclosed herein is worn on the wearer's wrist and “top” refers to the side facing away from the wearer's wrist.

FIG. 1A illustrates a first view of an example wearable device including a physiological parameter measurement sensor or module worn on a wrist using straps.

FIG. 1B illustrates a second view of the example wearable device of FIG. 1A worn on the wrist.

FIG. 1C illustrates an example fingertip sensor that can be coupled to the wearable device of the present disclosure.

FIG. 1D illustrates a top perspective view of the example wearable device of FIGS. 1A-1C with a partial view of the straps.

FIG. 1E illustrates a bottom perspective view of the example wearable device of FIG. 1D.

FIG. 1F illustrates a side view of an example wearable device without the straps when the device is interfacing with a wearer's skin.

FIG. 1G illustrates a top perspective view of the example wearable device of FIG. 1F.

FIG. 1H illustrates a bottom perspective view of an example wearable device.

FIG. 1I illustrates a perspective view of an example strap configured to secure the wearable device disclosed herein to a wearer's wrist.

FIGS. 1J-1K illustrate perspective views of an example wearable device and physiological sensor or module removably secured to a wearable apparatus.

FIG. 2 is a diagram illustrating schematically a network of non-limiting examples of devices that can communicate with the wearable device disclosed herein.

FIG. 3 illustrates an example display of physiological parameter measurements on the wearable device disclosed herein.

FIG. 4 illustrates an example physiological parameter measurement module of the wearable device.

FIG. 5A illustrates a side view of an example wearable device incorporating an example physiological parameter measurement module.

FIG. 5B illustrates a cross-sectional view of the example wearable device of FIG. 5A.

FIG. 5C illustrates a perspective view of the wearable device of FIG. 5A.

FIG. 5D illustrates a bottom view of the wearable device of FIG. 5A.

FIG. 6 illustrates schematically arteries and capillaries of a human hand and a proximal portion of a human forearm.

FIG. 7A illustrates a schematic system diagram of a wearable device including a physiological parameter measurement module.

FIG. 7B illustrates a partially exploded view of an example wearable device.

FIG. 7C illustrates an example light transmissive cover of the physiological parameter measurement module of FIG. 7B.

FIG. 7D illustrates an exploded view of ECG electrodes, light transmissive cover(s), and a opaque frame of the physiological parameter measurement module of FIG. 7B.

FIG. 7E illustrates a bottom perspective view of a physiological parameter measurement module incorporating the ECG electrodes, light transmissive cover(s), and a opaque frame of FIG. 7C or 7D.

FIG. 7F illustrates a top perspective view of the example physiological parameter measurement module of FIG. 7E.

FIGS. 7G and 7H illustrate schematically top and bottom views of an example device processor board of the wearable device disclosed herein.

FIGS. 8A and 8B illustrate schematically top and bottom views of an example sensor or module processor board of an example physiological parameter measurement module.

FIGS. 8C-8E illustrate various view of bonding of detectors to a PCB substrate of a physiological parameter measurement module.

FIG. 8F illustrates a perspective view of a PCB substrate of a physiological parameter measurement module with different wire bonding arrangements than shown in FIGS. 8C-8E.

FIGS. 9A and 9B illustrate light diffusing material fill channels and air venting channels in a opaque frame of an example physiological parameter measurement module.

FIG. 10 illustrates a longitudinal cross-sectional view of an example physiological parameter measurement module and example light paths between emitters and detectors of the module.

FIG. 11A illustrates a schematic system diagram of an example wearable device including a physiological parameter measurement module.

FIG. 11B illustrate a schematic diagram of an example device processor shown in FIG. 11A.

FIG. 11C illustrates a schematic system diagram of an example sensor or module processor shown in FIG. 11A.

FIG. 11D illustrates a block diagram of an example front end circuitry of the sensor or module processor of FIG. 11C.

FIG. 12A illustrates a bottom view of an example physiological parameter measurement module with first and second ECG electrodes.

FIG. 12B illustrates a top perspective view of the example wearable device including a third ECG electrode.

FIG. 12C illustrates a partial top perspective view of the example wearable device of FIG. 12B with the third ECG electrode shown as transparent to illustrate contact springs underneath the third ECG electrode.

FIG. 13A illustrates an example block diagram of LED drive circuitry of the physiological parameter measurement module disclosed herein.

FIG. 13B illustrates an example block diagram of emitters circuitry of the physiological parameter measurement module disclosed herein.

FIG. 13C illustrates an example block diagram of detectors circuitry of the physiological parameter measurement module disclosed herein.

FIG. 13D illustrates an example block diagram of temperature sensors circuitry of the physiological parameter measurement module disclosed herein.

FIGS. 14A and 14B are example block diagrams illustrating signal processing of a conventional plethysmograph sensor.

FIGS. 15A and 15B illustrate example schematic input and output flow diagrams of a physiological parameter measurement module disclosed herein.

FIG. 15C illustrates an example schematic input and output flow diagram of the gyroscope and accelerometer of a physiological parameter measurement module disclosed herein.

FIG. 15D illustrates an example schematic block diagram for determining pulse rate using a physiological parameter measurement module disclosed herein.

FIG. 15E illustrates an example decision logic for determining pulse rate using a physiological parameter measurement module disclosed herein.

FIG. 15F illustrates an example schematic input and output flow diagram for determining oxygen saturation using a physiological parameter measurement module disclosed herein.

FIG. 15G illustrates an example decision logic for determining oxygen saturation using a physiological parameter measurement module disclosed herein.

FIG. 16A illustrates schematically an example plethysmograph sensor arrangement on a sensor or module processor board of a physiological parameter measurement module of a wearable device.

FIG. 16B illustrates a bottom view of an example physiological parameter measurement module incorporating the plethysmograph sensor arrangement of FIG. 16A.

FIG. 16C illustrates a side view of the example physiological parameter measurement module of FIG. 16B.

FIG. 16D illustrates a bottom perspective view of the example physiological parameter measurement module of FIG. 16B.

FIG. 16E illustrates a bottom view of a variation of the example physiological parameter measurement module of FIG. 16B including ECG electrodes.

FIG. 16F illustrates a side view of the example physiological parameter measurement module of FIG. 16E.

FIG. 16G illustrates a bottom perspective view of the example physiological parameter measurement module of FIG. 16E with the opaque frame and light transmissive cover hidden to show ECG electrodes assembled with the sensor or module processor board.

FIG. 17A illustrates a bottom perspective view of an example physiological parameter measurement module incorporating the plethysmograph sensor arrangement of FIG. 16A.

FIG. 17B illustrates a bottom view of the example physiological parameter measurement module of FIG. 17A.

FIG. 17C illustrates a side view of the example physiological parameter measurement module of FIG. 17A.

FIG. 18A illustrates schematically an example plethysmograph sensor arrangement on a sensor or module processor board of a physiological parameter measurement module of a wearable device.

FIG. 18B illustrate schematically an example plethysmograph sensor arrangement on a sensor or module processor board of a physiological parameter measurement module of a wearable device.

FIG. 19A illustrate schematically an example plethysmograph sensor arrangement on a sensor or module processor board of a physiological parameter measurement module of a wearable device.

FIG. 19B illustrates a bottom view of an example physiological parameter measurement module incorporating the plethysmograph sensor arrangement of FIG. 19A.

FIG. 19C illustrates a side view of the physiological parameter measurement module of FIG. 19B.

FIG. 20A illustrates a bottom view of an example physiological parameter measurement module of a wearable device as worn on a schematic representation of a wearer's wrist.

FIG. 20B illustrates a side view of the physiological parameter measurement module of FIG. 20A.

FIGS. 20C and 20D illustrate exploded views of the physiological parameter measurement module of FIG. 20A.

FIG. 20E illustrates a first side view of an example wearable device incorporating the physiological parameter measurement module of FIGS. 20A-20D.

FIG. 20F illustrates a bottom view of the wearable device of FIG. 20E.

FIG. 20G illustrates a second side view of the wearable device of FIG. 20E.

FIG. 20H illustrates a third side view of the wearable device of FIG. 20E.

FIG. 20I illustrates a bottom perspective view of the wearable device of FIG. 20E.

FIG. 20J illustrates a top perspective view of the wearable device of FIG. 20E.

FIGS. 21A and 21B illustrate perspective views of an example physiological parameter measurement module with alternative light transmissive cover curvatures from the module in FIG. 20A.

FIG. 21C illustrates a longitudinal cross-sectional view of the physiological parameter measurement module of FIGS. 21A and 21B.

FIGS. 22A and 22B illustrate perspective views of an example physiological parameter measurement module with another alternative light transmissive cover curvatures from the module in FIG. 20A.

FIG. 22C illustrates a longitudinal cross-sectional view of the physiological parameter measurement module of FIGS. 22A and 22B.

FIG. 23A illustrates a bottom perspective view of an example wearable device incorporating the physiological parameter measurement module of FIGS. 20A-20D.

FIG. 23B illustrates a side view of the wearable device of FIG. 23A.

FIG. 23C illustrates a top perspective view of the wearable device of FIG. 23A.

FIG. 23D illustrates a top view of the wearable device of FIG. 23A.

FIG. 23E illustrates a bottom view of the wearable device of FIG. 23A.

FIG. 24A illustrates a bottom view of another example physiological parameter measurement module of a wearable device.

FIG. 24B illustrates a side view of the physiological parameter measurement module of FIG. 24A.

FIG. 25A illustrates a bottom view of another example physiological parameter measurement module of a wearable device.

FIG. 25B illustrates a side view of the physiological parameter measurement module of FIG. 25A.

FIG. 25C illustrates a first side view of another example wearable device incorporating the physiological parameter measurement module of FIGS. 25A-25B.

FIG. 25D illustrates a bottom view of the wearable device of FIG. 25C.

FIG. 25E illustrates a second side view of the wearable device of FIG. 25C.

FIG. 25F illustrates a top perspective view of the wearable device of FIG. 25C.

FIG. 25G illustrates a third side view of the wearable device of FIG. 25C.

FIG. 25H illustrates a bottom perspective view of the wearable device of FIG. 25C.

FIG. 26A illustrates schematically a microneedle inserted into skin of a wearer.

FIG. 26B illustrates schematically a microneedle patch coupled to a body of the wearable device disclosed herein.

FIG. 26C illustrates schematically a microneedle patch coupled to a strap of the wearable device disclosed herein.

FIG. 26D illustrates schematically a simplified system diagram of the microneedle patch and the wearable device.

FIG. 27A illustrates a front view of an example aspect of a physiological parameter measurement sensor or module.

FIG. 27B illustrates an exploded view of an example aspect of a physiological parameter measurement sensor or module.

FIGS. 27C and 27D illustrate light diffusing material fill channels and air venting channels in an opaque frame of an example physiological parameter measurement sensor or module.

FIG. 27E illustrates an example physiological parameter measurement sensor or module and example light paths between emitters and detectors of the module.

FIG. 27F illustrates a perspective view of PCB substrate of a physiological parameter measurement sensor or module with example plethysmograph sensor arrangement.

FIG. 27G illustrates a longitudinal cross-sectional view of an example physiological parameter measurement sensor or module.

FIG. 27H illustrates a longitudinal cross-sectional, that is orthogonal to the view of FIG. 27G, of an example physiological parameter measurement sensor or module.

FIG. 27I illustrates a perspective view of PCB substrate of a physiological parameter measurement sensor or module with example plethysmograph sensor arrangement.

FIG. 27J illustrates a perspective cross-sectional view of an example physiological parameter measurement sensor or module.

FIGS. 27K-27L illustrate an example physiological parameter measurement sensor or module and example light paths between emitters and detectors of the module.

FIGS. 27M-27P illustrate an example physiological parameter measurement sensor or module and example light barriers or light blocks between emitter and detector chambers of the module.

FIG. 27Q illustrates an example physiological parameter measurement sensor or module and example light diffusing material and light transmissive lens(es) or cover(s).

FIG. 28 shows a block diagram illustrating an example aspect of the wearable device in communication with an external device via a network.

FIG. 29 illustrates an example dashboard user interface of the health application.

FIGS. 30A-30D illustrate example spot check monitoring user interfaces of the health application.

FIGS. 30E-30H illustrate example continuous monitoring user interfaces of the health application.

FIGS. 31A-31D illustrate example measurement settings user interfaces of the health application.

FIG. 32 illustrates an example activity user interface of the health application.

FIGS. 33A-33C illustrate example workout user interfaces of the health application.

FIGS. 34A-34C illustrate example sleep user interfaces of the health application.

FIGS. 35A-35B illustrate example history user interfaces of the health application.

FIG. 36A illustrates an example aspect of a wearable device with a display screen.

FIGS. 36B-36E illustrate example display screens of a wearable device.

FIG. 37A illustrates an example aspect of a wearable device with a display screen.

FIGS. 37B-37E illustrate example display screens of a wearable device.

FIG. 38A-38B illustrate example display screens of a wearable device.

FIG. 39 is a flowchart illustrating an example process for performing a monitoring operation of the wearable device.

FIG. 40 illustrates a block diagram of an example computing device that may implement one or more aspects of the present disclosure, according to various aspects of the present disclosure.

### DETAILED DESCRIPTION

Although certain aspects and examples are described below, those of skill in the art will appreciate that the disclosure extends beyond the specifically disclosed aspects and/or uses and obvious modifications and equivalents thereof based on the disclosure herein. Thus, it is intended that the scope of the disclosure herein disclosed should not be limited by any particular aspects described below.

### Overview of Wearable Device Including a Physiological Parameter Measurement Sensor or Module

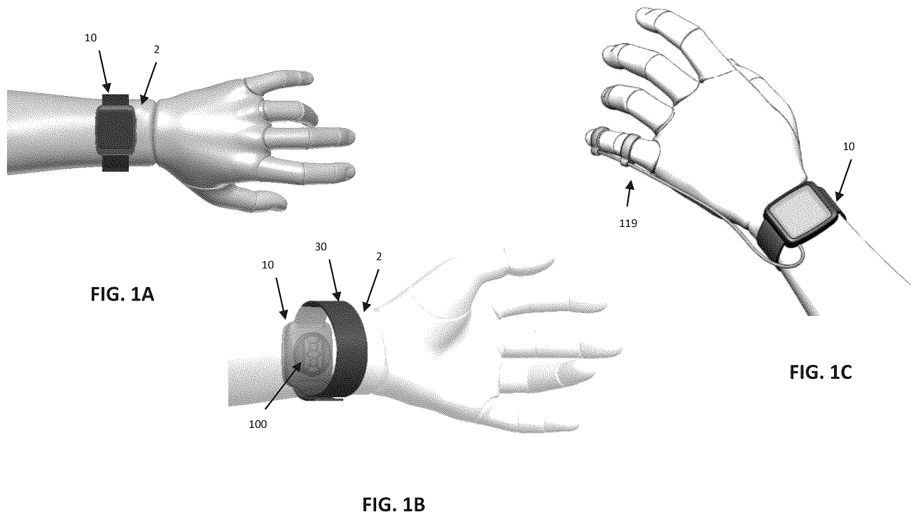

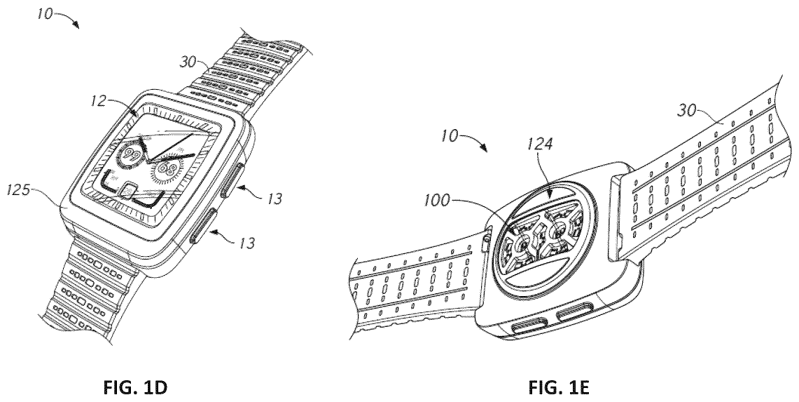

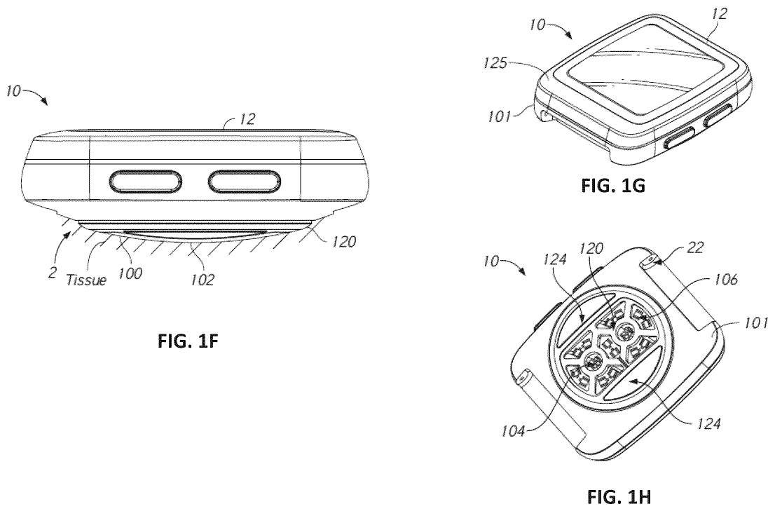

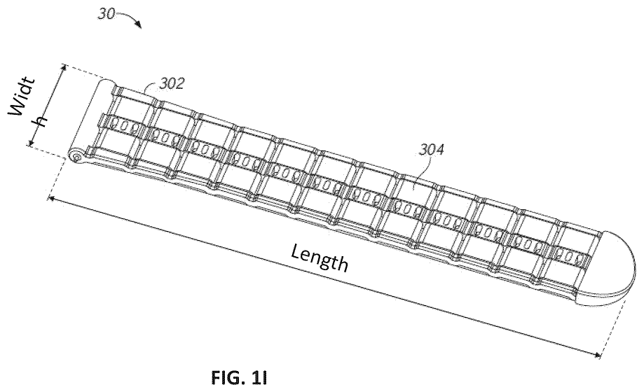

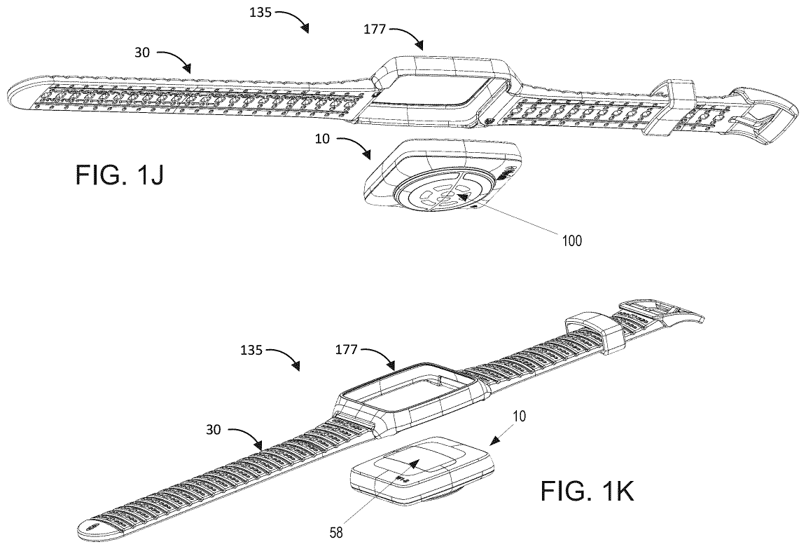

Daily use of a wearable healthcare monitoring device, which can include oximetry- or plethmosmograph-based and/or ECG physiological parameters, can be beneficial to the wearer. The device, such as a device 10 as shown in FIGS. 1A-1H, can be a wristwatch incorporating a physiological parameter measurement sensor 100 or a wrist-worn physiological parameter measurement sensor with built-in watch or time-indicating functions. The device 10 can include an adjustable strap 30. Accordingly, the wearer needs not wear an additional sensor when going about daily activities and the appearance of the device attracts less attention from the general public so that the wearer may feel less self-conscious about wearing a pulse oximeter sensor on the wearer's body. The wearer can also connect additional sensors (for example, a fingertip plethysmograph sensor shown in FIG. 1C) and/or other physiological monitoring devices to the wearable device to expand the functionality of the wearable device.

The wearer can be informed of physiological parameters, such as vital signs including but not limited to heart rate (or pulse rate), and oxygen saturation by the wearable device 10. The device 10 can display one or more of the measured physiological parameters on its display 12. The information can be helpful in providing feedback to the wearer and/or a third party user, for example, a healthcare professional or the wearer's family member, when the wearer is exercising, or otherwise for warning the wearer of possible health-related conditions, including but not limited to changes in the wearer's physiological parameters in response to medication that is being administered to the wearer.

As shown in FIGS. 1A-1H, the wearable device 10 can be a watch, which can include a physiological parameter measurement sensor or module 100 configured to measure an indication of the wearer's physiological parameters, which can include, for example, pulse rate, respiration rate, oxygen saturation (SpO2), Pleth Variability Index (PVI), Perfusion Index (PI), Respiration from the pleth (RRp), hydration, glucose, blood pressure, and/or other parameters. The physiological parameter measurement sensor or module 100 can be an optical sensor. Additionally, the sensor or module 100 can optionally calculate a wellness index based on more than one individual physiological parameter measured by the module and/or received by the sensor or module 100 based on externally connected sensors and/or patient monitoring devices. The sensor or module 100 can perform intermittent and/or continuous monitoring of the measured parameters. The sensor or module 100 can additionally and/or alternatively perform a spot check of the measured parameters, for example, upon request by the wearer.

As shown in FIGS. 1E and 1H, a bottom side of a device (or watch) housing 101 can include an opening sized to retain the physiological parameter measurement module 100 while still allowing the tissue-facing surface of the sensor or module 100 to be exposed. The retaining of the sensor or module 100 in the device housing 101 can be aided by any suitable retaining mechanisms. As shown in FIGS. 1F and 1H, the physiological parameter measurement module 100 can include a skin-interfacing light transmissive cover 102 that encloses a plurality of light emitters 104 (such as LEDs) and one or more photodetectors (also referred to as “detectors”) 106. Additionally, the sensor or module 100 can optionally include an electrocardiogram (ECG) sensor, which can include a plurality of ECG electrodes 124, 125. As shown in FIGS. 1G and 1H, some of the ECG electrodes 125 can be located away from the sensor or module 100 and some of the ECG electrodes 124 can be located on the sensor or module 100. The cover 102 can include a plurality of lenses or covers or a single construct of lens or cover. The physiological parameter measurement module 100 is designed to reduce noise in the signals detected by the detectors 106, for example, by reducing mixing of the emitted light and the reflected light using light barriers that are substantially opaque. As shown in FIG. 1F, the light barrier 120 can include a first light barrier which can be placed between the emitters and the detectors of the sensor or module 100. The first light barrier can extend (for example, entirely extend) along an inner portion of the cover 102. The first light barrier can also suppress light emitted by the emitters at an angle. The sensor or module 100 can include additional light barriers, including for example, a side perimeter wall and additional light barriers to separate the detectors from the emitters, and/or separate different detector groups from one another.

FIG. 1F illustrates the device 10 being worn on the wrist 2 of the wearer, with the physiological parameter measurement module 100 facing the wrist 2. The physiological parameter measurement module 100 on the device 10 is designed so as to reduce and/or eliminate a gap between a surface of the physiological parameter measurement module 100 and the wearer's skin at the measurement site where the device 10 is worn. At the wrist, if the device 10 is worn too loosely (which can be the case when the device 10 is able to slide over the skin when the device 10 is moved), the gap between the tissue-facing surface of the physiological parameter measurement module 100 and the wearer's skin can cause inaccurate measurements. This is because the gap can result in both light-piping and in the emitted light not penetrating deep enough into the wearer's tissue, for example, by going no deeper than within a top skin layer (for example, the epidermis) of the wearer's tissue, which typically does not have any blood vessels present. Therefore, light cannot reach and or interact with tissues, such as the arterial blood in the dermis, located below the top skin layer. The gap can also result in loss of the attenuated and reflected light through the gap so that less of the attenuated and reflected light can arrive at the detectors 106.

The tightness of the device 10 on the wearer's body (for example, the wrist) can be adjusted by adjusting any suitable strap(s) 30 used to secure the device to the wearer's body. The strap(s) can be connected to the device 10 using any suitable strap connections 22. For example, the strap connections 22 can be compatible with third party watch bands, wearable blood pressure monitors, and/or the like. As shown in FIG. 1I, an example strap 30 can be stretchable and evenly distribute the pressure of the device 10 around the wrist so as to provide better contact between the sensor or module 100 and the wrist 2 while not compromising the comfort of the wearer and/or reducing the blood flow across the wrist 2 in a way that reduces the accuracy of the measurement by the sensor or module 100. As shown in FIG. 1L, a rubber base 302 can be molded through a plurality of metal loops 304 arranged along a length of a strap 30 to form the strap 30. The metal loops 304 can include a thin (for example, less than about 1 mm) wall of metal forming a closed loop with a through-hole in a direction generally transverse to the length (that is, along a width) of the strap 30 and perpendicular to a thickness of the strap 30. During the overmolding process, the rubber material can fill up or substantially fill up the space in the through-hole. The metal loops 304 can be arranged in two rows along the length of the strap 30. Alternatively, the metal loops can include a partial loop with an opening, or the strap may include more than one partial metal loop snapped onto each other around the rubber base. Additional details of the strap 30 are described in U.S. Provisional Application No. 63/068,256, filed Aug. 20, 2020 and titled “WEARABLE PHYSIOLOGICAL MONITORING DEVICE WITH ADJUSTABLE STRAPS”, the entirety of which is incorporated herein by reference.

Additionally, the gap between a surface of the physiological parameter measurement module 100 and the wearer's skin at the measurement site can be reduced by the design of the light transmissive cover 102. As shown in FIG. 1F, a cover 102 of the physiological parameter measurement module 100 can include a convex curvature or convex protrusion on its skin-interfacing cover 102. As will be described in greater detail below, the curvature of the cover 102 of the sensor or module 100, which can include a plurality of lenses or covers or a single lens or cover, can be discontinuous or continuous.

As shown in FIG. 1F, when the device 10 is worn by the wearer, the convex cover 102 can be pressed onto the skin and the tissue 2 of the wearer can conform around the convex curvature. The contact between the convex cover 102 and the tissue 2 of the wearer can leave no air gaps between the tissue 2 and the convex cover 102. And as the emitters and/or detectors can be surrounded by a light-diffusing material (as will be described below), the physiological parameter measurement module 100 may leave no air gap between the tissue 2 and any of the emitters and/or detectors. Optionally, certain portion(s) of the cover 102 can protrude more into the skin than the remainder of the cover. The pressure exerted by the curvature of the cover 102 on the skin and/or the absence of air gap can increase a light illuminated and/or detection area, improve the optical coupling of the emitted light and the blood vessels and/or of the reflected light and the detectors, reduce light piping, and/or reduce stagnation of the blood. The cover curvature can be configured so as to balance the pressure needed to improve contact between the cover 102 and the skin, and the comfort of the wearer.

FIGS. 1J-1K illustrate perspective views of an example wearable device 10 removably secured to a wearable apparatus 135. The wearable apparatus 135 includes one or more straps 30 and a frame 177. The wearable device 10 may include a physiological sensor or module 100 and an electrode 58. The electrode may be a positive electrode. In some aspects, the wearable device 10 may include a screen or display. In some aspects, the wearable device 10 may not include a screen or display.

The device 10 may be removably secured to the frame 177. The frame 177 may hold the device 10 within the frame 177 via force-fit, friction-fit, snap-fit, or the like. The straps 30 may secure the apparatus 135 to a body part of a wearer. The wearable apparatus 135 may be configured to hold the wearable device 10 (e.g., the sensor or module 100) in contact with the skin of a wearer when the device 10 is secured within the frame 177 and the wearable apparatus 135 is secured to the body of a wearer. The wearable apparatus 135 (e.g., straps 30) may be sized and/or shaped to secure to a wrist of a wearer. In some aspects, the wearable apparatus 135 (e.g., straps 30) may be sized and/or shaped to secure to other portions of a wearer's body, including an ankle, a leg, an arm, a finger, a chest, a torso, a stomach, a back, a neck, a head, a forehead, as non-limiting examples.

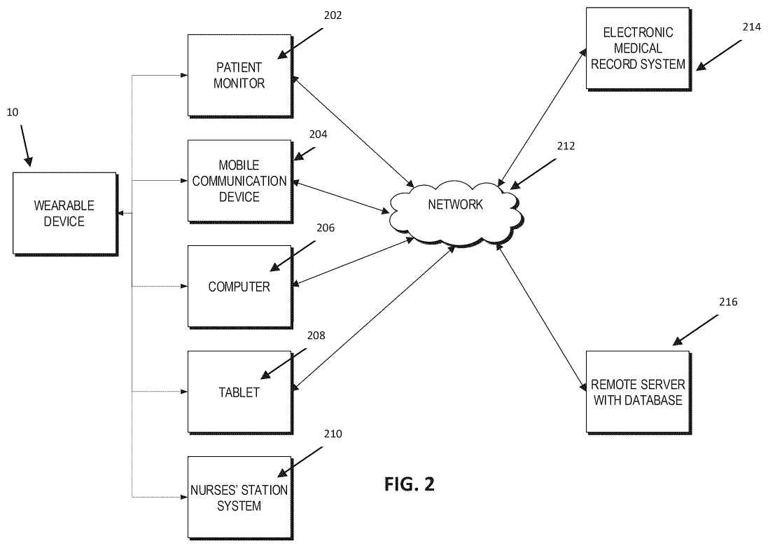

The wearable device 10 can be used in a standalone manner and/or in combination with other devices and/or sensors. As shown in FIG. 2, the device 10 can connect (for example, wirelessly) with a plurality of devices, including but not limited to a patient monitor 202 (for example, a bedside monitor such as Masimo's Radical-7®, Rad-97® (optionally with noninvasive blood pressure or NomoLine capnography), and Rad-8® bedside monitors, a patient monitoring and connectivity hub such as Masimo's Root® Platform, any handheld patient monitoring devices, and any other wearable patient monitoring devices), a mobile communication device 204 (for example, a smartphone), a computer 206 (which can be a laptop or a desktop), a tablet 208, a nurses' station system 201, glasses such as smart glasses configured to display images on a surface of the glasses and/or the like. The wireless connection can be based on Bluetooth technology, near-field communication (NFC) technology, and/or the like. Additionally, the wearable device 10 can connect to a computing network 212 (for example, via any of the connected devices disclosed herein, or directly). The network 212 may comprise a local area network (LAN), a personal area network (PAN) a metropolitan area network (MAN), a wide area network (WAN) or the like, and may allow geographically dispersed devices, systems, databases, servers and the like to connect (e.g., wirelessly) and to communicate (e.g., transfer data) with each other. The wearable device 10 can establish connection via the network 212 to one or more electronic medical record system 214, a remote server with a database 216, and/or the like.

Optionally, the device 10 can be integrated with more sensors and/or configured to connect to a plurality of external sensors, wirelessly or with a connecting cable. The connecting cable can be a universal connector configured to connect to any of the medical devices and/or sensors disclosed herein to provide communication between the wearable device 10 and the connected medical devices and/or sensors. The cable can optionally include a board-in-cable device that includes its own processor, but may not include its own display.

The device 10 can act as hub for the external sensors, for example, the sensors described in U.S. Patent Publication No. 2020/0138288, published on May 7, 2020 (the entirety of which is hereby incorporated herein by reference). The sensors described in U.S. Patent Publication No. 2020/0138288 can collect patient physiological data and provide power for a reusable pairing device. The reusable pairing device can establish wireless communication with a patient monitoring device. The wearable device 10 can replace the patient monitoring device in U.S. Patent Publication No. 2020/0138288. As another example, the device 10 can replace a patient monitor device described in U.S. Patent Publication No. 2020/0329993, published on Oct. 22, 2020, the entirety of which is hereby incorporated herein by reference. By replacing the patient monitor device in U.S. Patent Publication No. 2020/0329993, the wearable device 10 performs all the computations based on the sensor data so that the connected external sensors, for example, the ECG sensors disclosed in U.S. Patent Publication No. 2020/0329993, do not require heavy computing power.

The device 10 can include open architecture to allow connection of third party wireless sensor, and/or allow third party access to a plurality of sensors on the wearable device 10 or connected to the wearable device 10. The plurality of sensors can include, for example, a temperature sensor, an altimeter, a gyroscope, an accelerometer, emitters, LEDs, etc. Third party applications can be installed on the wearable device 10 and can use data from one or more of the sensors on the wearable device 10 and/or in electrical communication with the wearable device.

Optionally, the wearable device 10 can communicate with any other suitable noninvasive sensor, such as an acoustic sensor, a blood pressure sensor, temperature sensor, movement sensor, ECG sensor, etc. Examples of some of these devices include Masimo's Radius PPG™ sensor, Radius T™ sensor, and Centroid™ sensor, or otherwise. One or more of those sensors, for example, the Centroid™ sensor, can be used for stroke detection. The wearable device 10 can output an alert of stroke detection of the wearer and/or automatically initiate communication with a first respondent and/or the wearer's guardian or next-of-kin upon stroke detection.

The wearable device 10 can optionally communicate with chemical sensors, which can detect, for example, chemicals on the wearer's skin, and/or sweat, and/or the odor of certain chemicals in the air. The chemical sensors can include electrochemical sensors or any other suitable types of chemical sensors. A chemical sensor configured to analyze compositions of sweat can output measurements aiding the wearable device 10 in detecting stress and/or the wearer's hydration status. The wearable device 10 can optionally communicate with a skin impedance sensor, which can be used for monitoring the hydration status of the wearer.

Another example sensor that can be integrated into or connected to the device 10 and/or the sensor or module 100 can include a toxin and/or radiation detector configured to detect toxins in air (for example, pollution or contaminant particulates, carbon monoxide, smoke, and the like in the air). The toxin detection can aid care providers and/or firefighters who wear the device 10. Alternatively, the device 10 can be connected wirelessly to an external toxin and/or radiation detector. The toxin and/or radiation detector can be used with a smart mask. For example, the external toxin and/or radiation detector can be located on the mask, which can allow the mask to output a warning to the wearer of the mask when the mask filter or cartridge needs replacement.

Optionally, the wearable device 10 can communicate with glucose monitors, which can be invasive or minimally invasive such as finger prick type of glucose monitors, or a continuous noninvasive glucose monitor. The wearable device 10 can receive and display the wearer's glucose level from the glucose monitor. The wearable device 10 can also optionally be in communication with an insulin pump. The wearable device 10 can send a control signal to dispense insulin from the insulin pump to the wearer based on the monitored glucose level of the wearer.

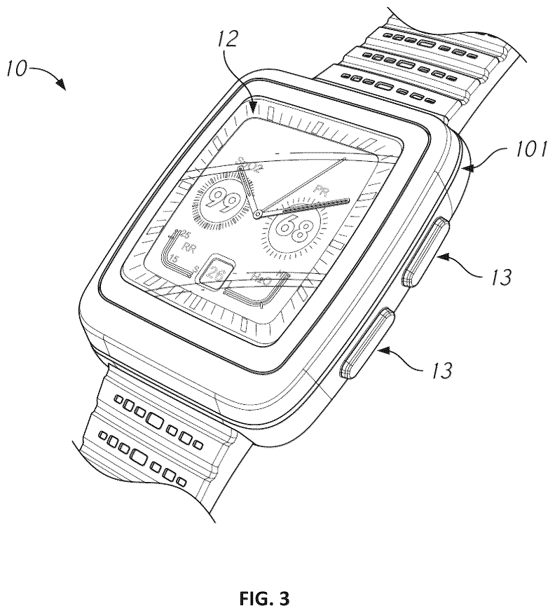

As shown in FIG. 3, the device 10 can include a display screen 12 positioned at a top side of the device housing 101. In addition to time and date indicators, one display layout (for example, the default display layout) of the display screen 12 can display the wearer's SpO2 measurement, the pulse rate (PR) measurement, the respiration rate (RR) measurement, and/or hydration status (H2O). The format of the measurement displayed is not limiting. For example, some measurements, such as the SpO2 measurement and the PR measurements, can be displayed as numerical values. As another example, some measurements, such as the RR measurements and hydration status, can be displayed as a sliding scale. In the illustrated example, the hydration status can be displayed as having three levels from low (L) to high (H). In the illustrated example, the respiration rate can be displayed as ranging from 5 bpm to 25 bpm. The wearer can optionally view individual display layouts for each measurements or a group of measurements by tapping on the display screen 12, which can be a touch screen, and/or pressing a button on the device 10. Each of the measurements can be displayed constantly, at certain intervals, and/or upon receiving instructions for display (for example, by the wearer tapping on the display screen 12 and/or pressing a button on the device 10). Each of the measurements can be configured to be displayed with different or the same frequencies. Time and certain physiological parameters (for example, SpO2 and pulse rate) can be immediately and/or intermittently available, and/or continuously measured (for example, at least every 5 to 10 measurements per minute or more) and the displayed values constantly updated. Optionally, the display can further show a trend line for some parameters, such as SpO2 and pulse rate. In one example, the display of the wearable device can be configured to display only time, SpO2, and pulse rate.

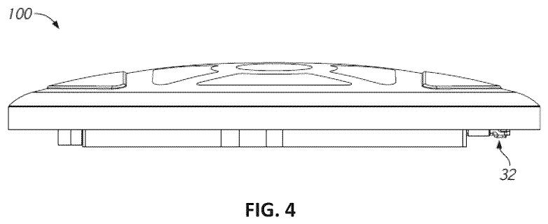

As shown in FIG. 4, the physiological parameter measurement module 100 can be preassembled before being integrated into the device 10. The physiological parameter measurement module 100 can be characterized before being assembled with the rest of the device 10. The preassembled physiological parameter measurement module 100 can be secured within the device housing 101 using various mechanical assembly mechanisms, for example, one or more screws or other fasteners. The sensor or module 100 of a wearable device 10 can be interchangeable and be replaced without replacing the memory in the device 10. For example, the sensor or module 100 can include a quick-connect (and/or quick-release) feature for attaching the sensor or module 100 to the remainder of the device 10, such as being attachable to the device 10 by magnets. An electrical connection can be established between the physiological parameter measurement sensor or module processor board and the circuit of the rest of the device 10, including for example, a device processor and the display 12. Optionally, the electrical connection can include a connector 32 on the sensor or module 100. The connector 32 is configured to be electrically connected to a flex circuit. The wearable device 10 and the sensor or module 100 are portable and can be moved from place to place. As described above, the functionality of the wearable device 10 can be integrated and/or interchangeable with various other patient monitoring devices, displays, etc.

The sensor or module 100 can be applied to locations on the body other than the wrist. Alternatively or additionally, multiple modules 100 can be applied to different locations of the body of the wearer. Other types of straps or fastening mechanism may be used to attach the multiple modules 100 onto other parts of the body. The other types of straps or fastening mechanism can optionally include a power source (for example, battery) to power a module 100 that is not integrated into the wearable device 10, but may not have its own display. For example, an optical sensor can be placed on the wearer's neck to measure arterial and venous oxygen saturation, which can be transmitted to and displayed on the wearable device 10. The wearer can view his or her oxygen consumption information on the wearable device 10 based on the signals from the optical sensor on the neck and/or the signals from the sensor or module 100 that is located on the wearable device 10.

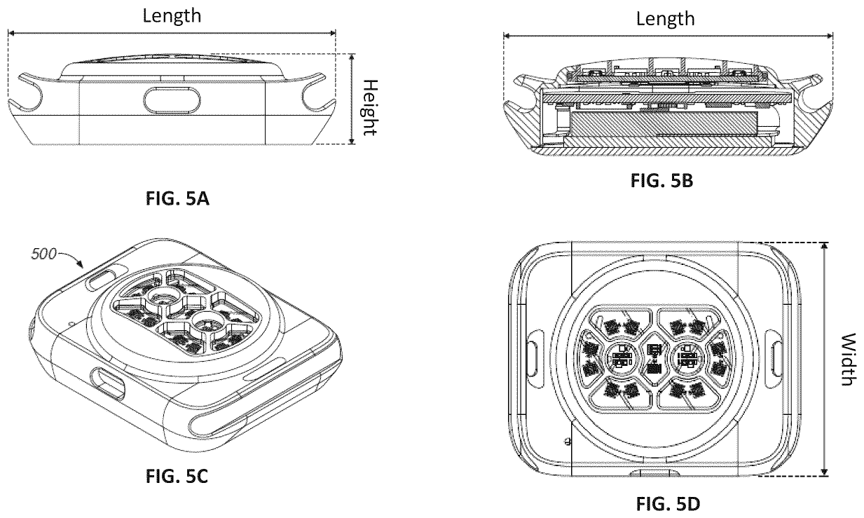

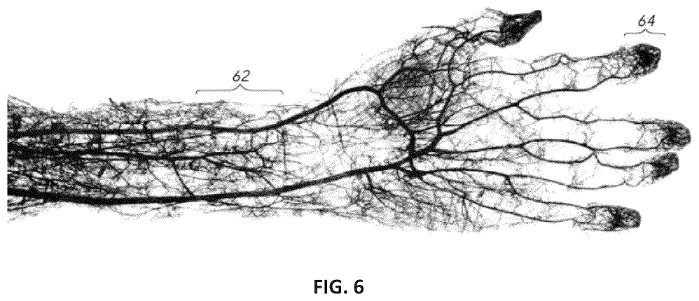

As shown in FIGS. 5A-5D, an example wearable device 500 can include a watch housing 501. Features of the device 500 can be incorporated into features of the device 10 and features of the device 10 can be incorporated into features of the device 500. The watch housing 501 can have a length, for example, between about 40 mm and 50 mm, or between about 42 mm and 46 mm. The watch housing can have a width, for example, between about 32 mm to about 40 mm, or between about 35 mm to about 38 mm. When fully assembled, the watch 500 can have a thickness or height, for example, between 10 mm to about 15 mm, or between 12 mm to about 14 mm.

As described above, the physiological parameter measurement module can include a plurality of emitters and a plurality of detectors. The emitters can transmit optical radiation of a plurality of wavelengths into a tissue site (near the wrist of the wearer) and the detectors can respond to the intensity of the optical radiation (which can be reflected from the tissue site) after absorption by pulsatile arterial blood flowing within the tissue site. In addition to the light being attenuated by blood in the arteries, light interaction also happens at the capillary level. Arteries are located deeper below the skin surface than the capillaries, requiring LED emitters of greater light intensity and thus greater power consumption in order for the emitted light to reach the arteries. Moreover, measuring the light intensities signal of the light after attenuation by blood in the artery requires more selective placement of the emitters and detectors directly above the arteries to capture the pulsation of the blood. The physiological parameter measurement module disclosed herein is designed to utilize attenuation by blood in the capillaries and is not reliant on the blood flow in arteries. The patient parameter measurements made by the module disclosed herein can be accurate enough for clinical use. The module disclosed herein can provide plethysmograph-based patient parameter measurements with an accuracy of within about 4% error, or about 2% error. As shown in FIG. 6, the wrist 62 has fewer capillaries per volume than the fingertip 64. Accordingly, the module is designed to have a width to provide greater coverage area of the wearer's wrist, which can boost the signal from the sensors located on the module (which will be described in greater detail below).

When measuring oxygen saturation based on attenuation by blood in the capillaries, it is desirable to avoid veins. Because venous blood contains less oxygen, intensity signals of light attenuated by venous blood can cause errant readings oxygen saturation measurement. Optionally, the sensor or module processor of the physiological parameter measurement modules disclosed herein can reduce the effect of pulsing vein on the signal by comparing the signals from the plurality of detectors to determine which detectors receive better and/or clearer signals and deactivating the detectors that are more likely to cover and/or be around the pulsing veins. The sensor or module processor can dynamically adjust which detectors to deactivate. Deactivating the detectors can include deactivating operation of that detector and/or ignoring signals from that detector.

Optionally, the sensor or module processor of the physiological parameter measurement module can map the physiological parameter measurements calculated from signals received at the detectors and/or clusters of detectors located at different regions of the module. Variations (for example, if outside a certain range) in the mapped measurements can be an indication that the pressure distribution of the wearable device on the body of the wearer is unbalanced, and therefore the pressure of the device on the wearer is either too high or too low and/or the wearable device is tilted on the wrist. The wearable device can output an instruction to the wearer to readjust the tightness of the straps and/or to re-center of the wearable device on the wrist. Variations (for example, if outside a certain range) in the mapped measurements can additionally or alternatively provide an indication that a certain detector or cluster of detectors is/are placed over a large pulsing vein as described above. Readings from that certain detector or cluster of detectors can be ignored or the detector(s) suspected to be cover a pulsing vein may be deactivated. When two or more physiological parameter measurements, such as oxygen saturation measurements, do not agree among two or more detectors (for example, having a variation exceeding a certain range), the sensor or module processor can use the higher or highest measurement value, or alternatively use a combination of the measurement values from the two or more detectors (for example, using one of the two measurement values at different times or otherwise).

Alternatively or additionally, the mapped measurements can be compared with experimentally determined data at the same detector location or detector cluster location. The experimentally determined data can be obtained using, for example, a conventional reflectance type pulse oximeter taped over the corresponding detector location, or any other suitable known methods for making the same measurements, including the same wrist-based sensor arrangements described herein. The comparison between the mapped measurements and the experimentally determined data can provide indication of whether the device has achieved a desired pressure on the body of the wearer, whether certain detectors and/or clusters of detectors are placed over or near a pulsing vein, which may interfere with the physiological parameter measurements, or otherwise. For example, if the difference between the mapped measurements and the experimental data at a certain location falls outside a predetermined range, the sensor or module processor can determine that pressure is too high or too low at that location, and/or that the pressure distribution over the body is not sufficiently balanced to make accurate measurements, and/or a detector or cluster of detectors is/are placed over the wearer's pulsing vein. The experimental data can be stored in a memory device of the sensor or module processor.

The comparison among the mapped measurements and/or between the mapped measurements and the experimental data can be performed when the wearer first puts on the device and/or at certain time intervals in the duration when the device is worn on the wearer. Additionally, running the comparison-based diagnostics can allow the sensor or module processor to determine, at the start of the measurement and/or dynamically during use of the device, which detector(s) provide the most accurate and/or reliable measurements.

### Various Example Components of the Wearable Device

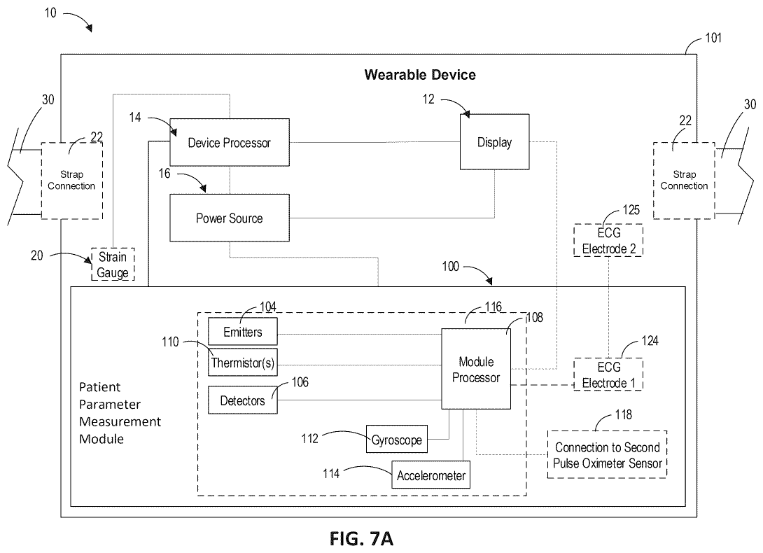

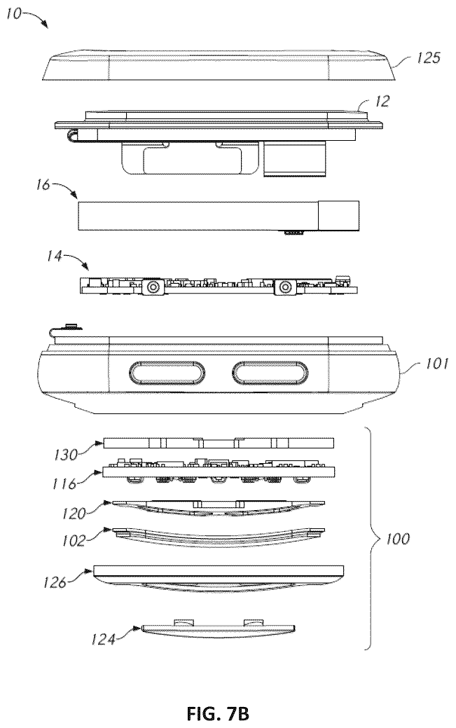

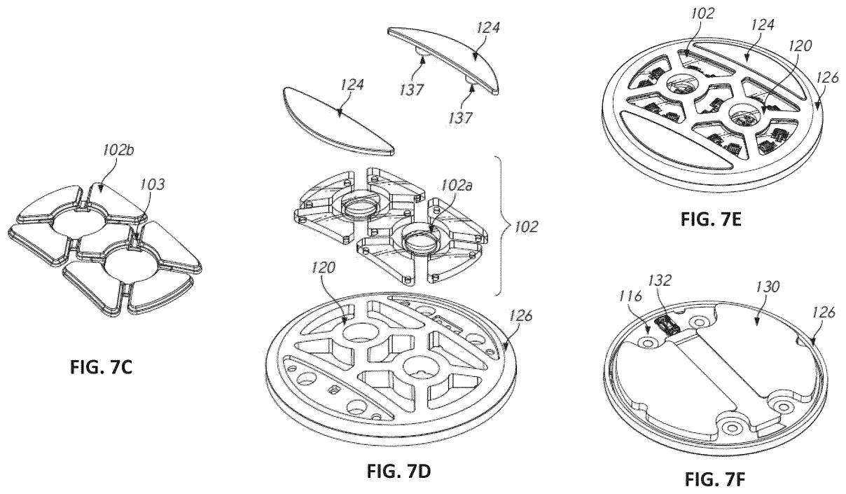

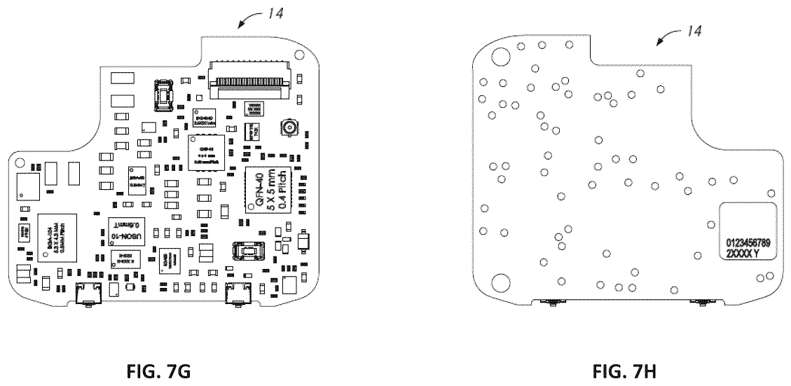

Components of the wearable device will now be described. As shown in FIGS. 7A and 7B, the device 10 can include its own device processor 14, which can be a digital/analog chip or other processor(s), such as a digital watch processor or a smartwatch processor. As shown in FIGS. 7B, 7G, and 7H, the device processor 14 can be located on a PCB. FIGS. 7G and 7H illustrate example layouts of the PCB for the device processor 14. As shown in FIGS. 7A and 7B, the device 10 can include a power source 16, which can be a battery, for powering the device processor 14, the display screen 12, and/or the physiological parameter measurement module 100. The battery 16 can last at least 10 hours, or at least 12 hours, or at least 14 hours, or at least about 16 hours after each charge, with continuous measurements and/or displaying of certain physiological parameters, such as SpO2 and pulse rate.

The device 10 can be configured to display time after the battery 16 has been depleted, even if other features (for example, measuring physiological parameters using the module) may not be available when the battery 16 has been depleted. Additionally, when the device 10 is used clinically, the display 12 can also continue displaying critical patient information (for example, the patient's name, date of admission, etc.) after the battery 16 has been depleted. The device 10 may include nonvolatile memory to store the critical patient information. The device 10 can include a dual-battery configuration with a main battery and a backup battery. Power management of the device 10 may switch automatically for the device 10 to be powered by the backup battery when the main battery has been depleted. The device can additionally or alternatively be configured to be solar-powered, for example, by including a solar panel on the dial or elsewhere of the wearable device 10. The display 12 of the device 10 can use e-ink or ULP (ultra low power screen) technology, which draws little amount of current for displaying information. The display 12 may automatically adjust the brightness, being brighter when outdoors and dimmer when indoors to further prolong battery life.

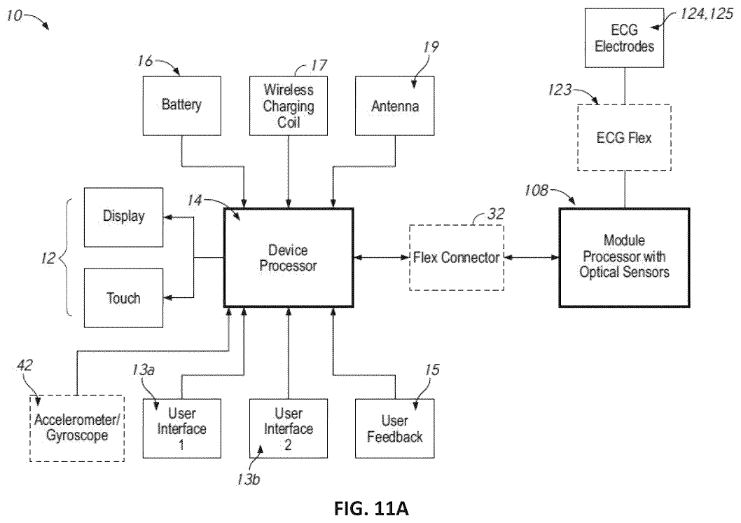

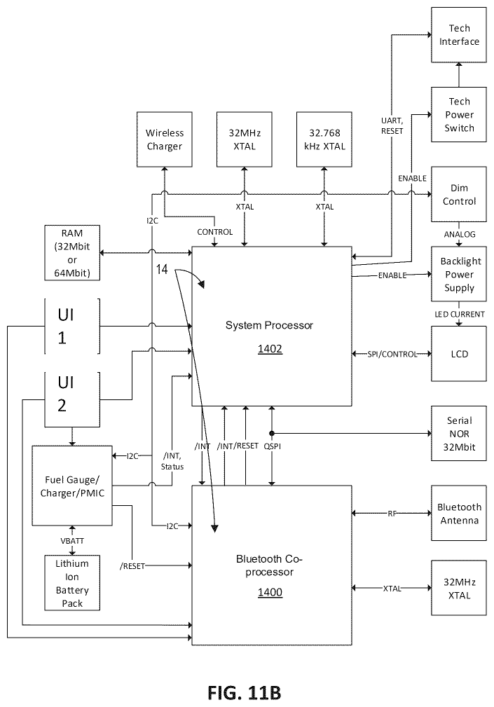

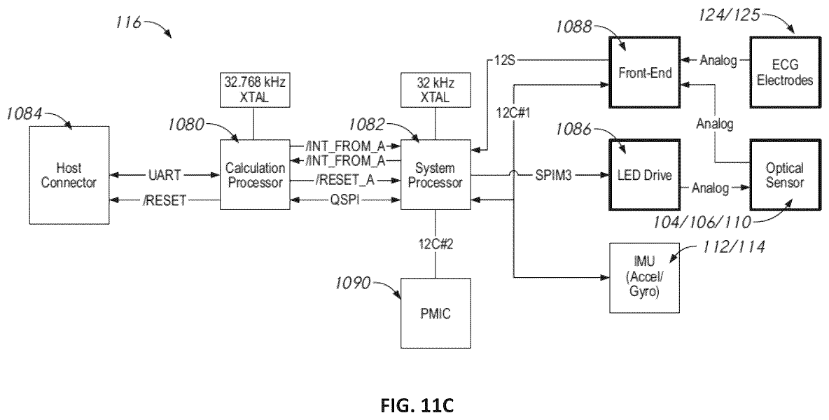

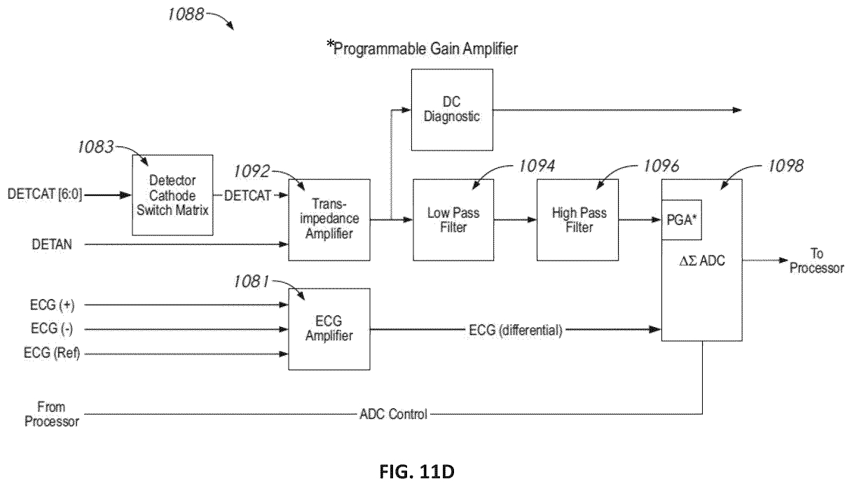

As shown in FIGS. 7A and 7B, the sensor or module 100 of the wearable device 10 can include a sensor or module processor 108 (which can include a memory and/or other electronics, such as shown in FIG. 11C). The sensor or module processor 108 can process signals from one or more of the sensors in the sensor or module 100 (or optionally other sensors in communication with the device 10) to determine a plurality of physiological parameters. All the processing of the raw sensor data of the sensors in communication (via a wired and/or wireless connection) with the sensor or module processor 108 is performed by the sensor or module processor 108. The sensor or module processor 108 can be configured to drive the emitters 104 to emit light of different wavelengths and/or to process signals of attenuated light after absorption by the body tissue of the wearer from the detectors 106. The sensor or module processor 108 can determine and output for display on the device display screen 12 the physiological parameters based on the detected signals. Optionally, the sensor or module 100 can send the signals from the detectors 106 (for example, preprocessed signals) to the device processor 14, which can determine and output for display the physiological parameters based on the detected signals. The absorption of light can be via transreflectance by the wearer's body tissue, for example, by the pulsatile arterial blood flowing through the capillaries (and optionally also the arteries) within a tissue site where the device 10 is worn (for example, the wrist). The sensor or module processor 108 can be located on a PCB 116, such as shown in FIG. 7B.

The sensor or module 100 can include more than one group or cluster of light emitters (such as LEDs) 104 and more than one group of photodetectors (also referred to as “detectors”) 106. Each group of emitters 104 can be configured to emit four (or three) different wavelengths described herein. The sensor or module 100 can include one or more thermistors 110 or other types of temperature sensors. The thermistor(s) 110 can be placed near one or more groups of emitters 104. There can be at least one thermistor 110 near each group of emitters 104. The thermistor(s) 110 can provide for wavelength correction of the light emitted by the emitters 104. Optionally, the thermistor(s) 110 can additionally measure a temperature of the wearer of the device 10. Optionally there can be one or more thermistors 110 located at other places of the sensor or module 100. The emitters 104, the thermistor(s) 110, and/or the detectors 106 can be positioned on the PCB 116.

As shown in FIG. 7A, the device 100 can include a gyroscope 112, an accelerometer 114, and/or other position and/or posture detection sensor(s). The gyroscope 112 and/or the accelerometer 114 can be in electrical communication with the sensor or module processor 108. The sensor or module processor 108 can determine motion information from signals from the gyroscope 112 and/or the accelerometer 114. The motion information can provide noise reference for analysis of the pleth information and other signal processing (for example, processing of ECG signals) performed by the sensor or module processor 108. The gyroscope 112 and/or the accelerometer 114 can be located on the PCB 116.

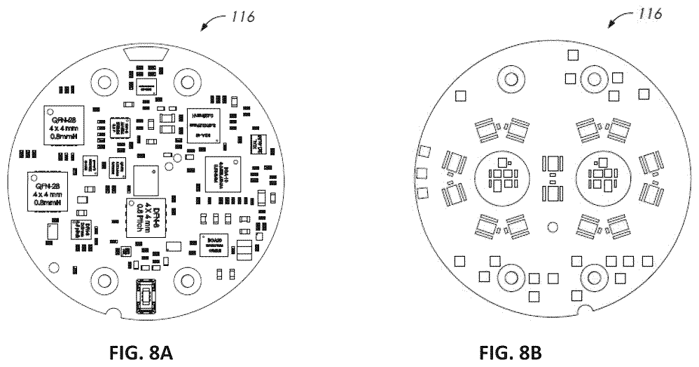

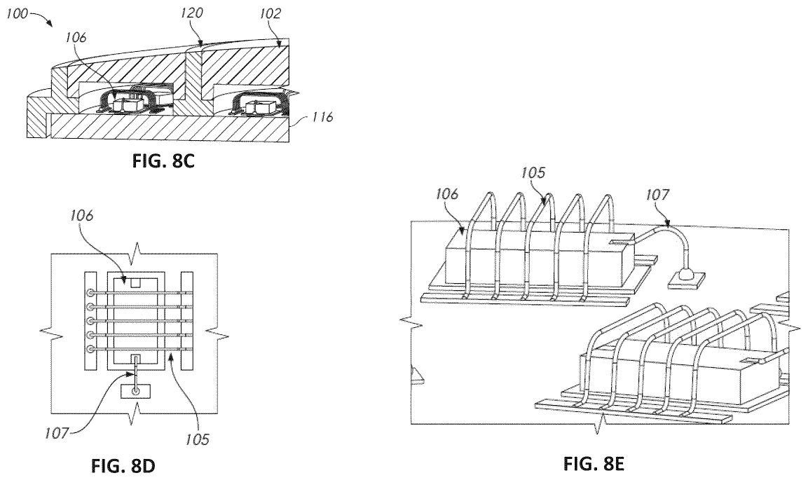

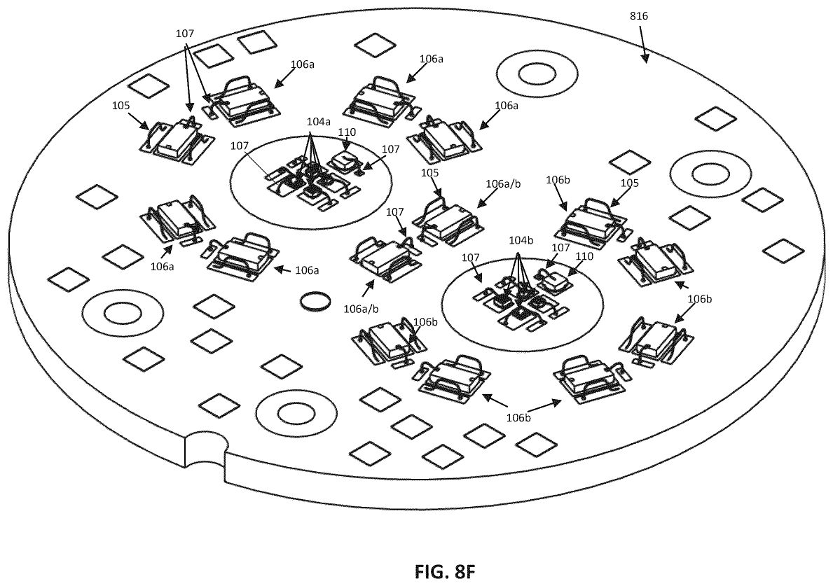

FIG. 8A illustrates example layouts of a top side of the PCB 116. FIG. 8B illustrates example layouts of a bottom side of the PCB 116. The first or bottom side of the PCB 116 can include the emitters 104, the detectors 106, the temperature sensor 110, and any other sensors, for example, the gyroscope, the accelerometer, and/or the like. FIGS. 8C-8E illustrate the detectors 106 being connected electrically to the PCB 116 via wire bonds 107. The module can include wires 105 extending over the detector 106 for shielding purposes. The number of wires 105 extending over the detector 106 may vary. The manner in which the wires 105 extend over the detector 106 may vary. The wires 105 may not extend all the way over the detectors 106 across the detector's width or length. For example, as shown in FIG. 8F, the detectors of detector groups 106a, 106b, 106a/b can each be connected electrically to the first side of the PCB 816 via wire bonds 107. A wire 105 can extend along each side of the detector for noise shielding. In the illustrated example, the wire 105 can extend along each long side of the detector. The wire 105 may extend parallel with the length of the detector. The wire 105 may not extend over the body of the detector 106a, 106b, 106a/b. In some aspects, a single wire 105 may extend along all four sides of the emitter for shielding. In some aspects, four wires 15 may each extend along one of the four sides of the emitter for shielding. In some aspects, a metal cover, such as a can shaped metal cover, may cover each of the detectors and/or emitters for shielding. The emitters in the emitter groups 104a, 104b can each be electrically connected to the first side of the PCB 816 via wire bonds 107. The thermistors 110 at each of the emitter groups 104a, 104b can be electrically connected to the first side of the PCB 816 via wire bonds 107. The detectors, emitters, and/or thermistor can alternatively be electrically connected to the PCB 116 via other suitable types of electrical connectors.

The second or top side of the PCB 116 can include the sensor or module processor 108 and other circuit hardware. The second side of the PCB 116 can be electrically noisy and is isolated from the sensors on the first side of the PCB 116 by the board. Electronics on the same side of the PCB 116 can be substantially entirely overmoulded to reduce or avoid components shifting in place or being damaged during use. On the second side of the PCB 116, which faces away from the light transmissive cover 102, the PCB 116 can be covered by melt plastic or other suitable electronics protective material 130, such as shown in FIGS. 7B and 7F. As shown in FIG. 7F, the electronic components on the second side of the PCB 116 can be generally sealed by the protective material 130 except that a connector 132 can extend from the second side of the PCB 116 and be exposed. The connector 132 can electronically connect the sensor or module 100 to circuitry of the wearable device 10.

Optionally, as shown in FIGS. 7A, 7B, and 7D, the device 10 can include an electrocardiogram (ECG) sensor including a plurality of electrodes 124, 125 configured to make contact with the wearer's skin. One or more ECG electrodes 124 may be located on the sensor or module 100 (such as shown in FIGS. 7B and 7E). One or more ECG electrodes 125 may be located elsewhere on the device (for example, an ECG electrode 125 can form a part of the housing of the wearable device 10 as shown in FIG. 7B). The ECG sensor can be in electrical communication with the sensor or module processor 108 via an ECG connector.

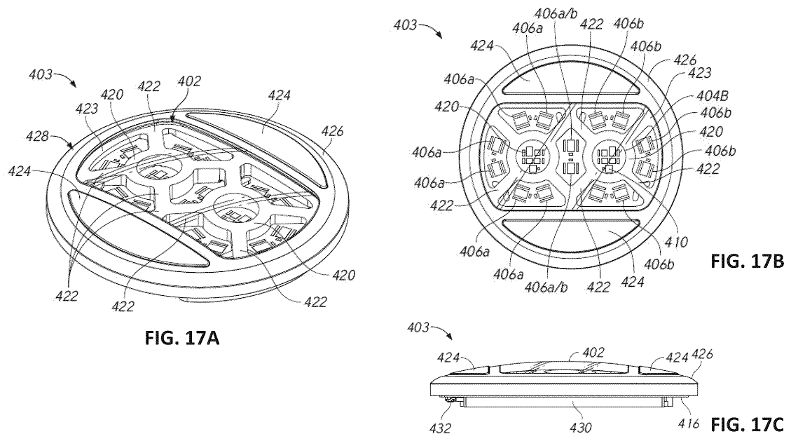

As shown in FIGS. 7B-7E, the physiological parameter measurement module 100 can include a skin-interfacing light transmissive cover 102 that encloses the first side of the PCB 116, which positions the plurality of light emitters 104 and detectors 106. The sensor or module 100 can include a light barrier construct 120 that is configured to divide the emitters 104 and the detectors 106 into different chambers such that light cannot travel or substantially cannot travel between the chambers. The light transmissive cover 102 can extend over the various emitter and detector chambers formed by the light barrier construct 120 and the PCB 116. The light transmissive cover 102 can include individual lenses or covers such as shown in FIG. 7D, a single lens or cover such as shown in FIGS. 17A-17C, or a combination of individual emitter chamber covering lenses or covers and a single lens or cover covering a plurality of detector chambers, such as shown in FIG. 7C. In the example lens or cover 102b shown in FIG. 7C, the individual lenses or covers that are configured to cover the detector chambers such as shown in FIG. 7D can be interconnected with bridging portions 103 between the detector chambers, forming a single piece of lens or cover. The lens or cover 102b can be combined with the lenses or covers 102a covering the emitter chambers to cover all the openings in the light barrier construct 120 for forming sealed emitter and detector chambers. The light barrier construct 120 can be overmoulded to the lens or cover 102b and the lenses or covers 102a. The lens or cover 102b may not be configured to cover the emitter chambers, which can be covered by individual lenses, so as to avoid any light traveling between an emitter chamber and a detector chamber.

As shown in FIG. 7B, the physiological parameter measurement module 100 can include an opaque frame 126. The opaque frame 126 can accommodate the light barrier construct 120. Alternatively, the opaque frame 126 and the light barrier construct 120 can form an integral piece, such as shown in FIG. 7D. The opaque frame 126 can include indentations having the shape and size to accommodate the ECG electrodes 124 or other components with a suitable shape and size. A front side of the electrodes 124 can have one or more posts 137 extending past openings in the opaque frame 126 into corresponding openings on the PCB 116. The posts 137 of the electrodes 124 can establish an electrical connection with the corresponding openings of the PCB 116. A plurality of screws (or other types of fasteners) can extend into the corresponding openings of the PCB 116 from the front side of the PCB 116 to secure the electrodes 124 to the sensor or module 100 by threadedly mating or otherwise with the posts 137. When a wearer puts the wearable device incorporating the sensor or module 100 onto the wearer's wrist, the electrodes 124 can make contact with the wearer's skin.

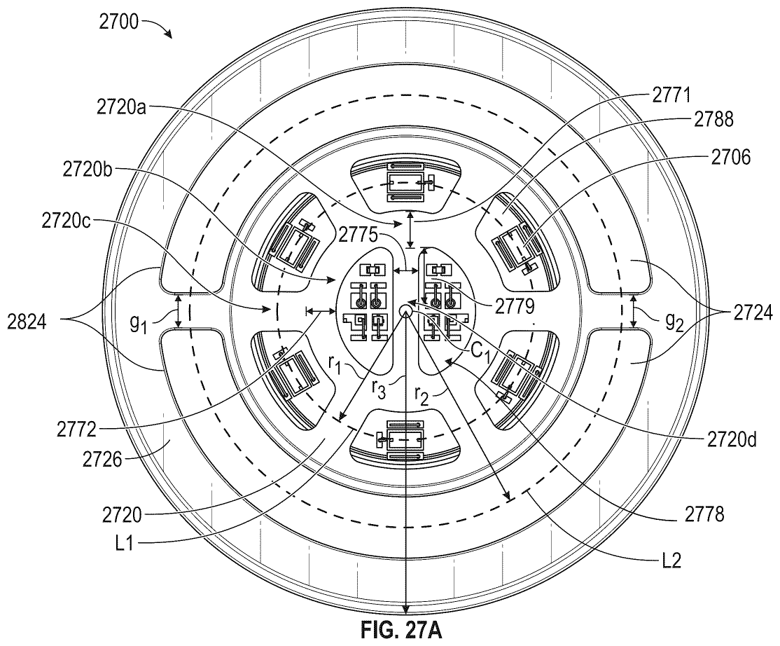

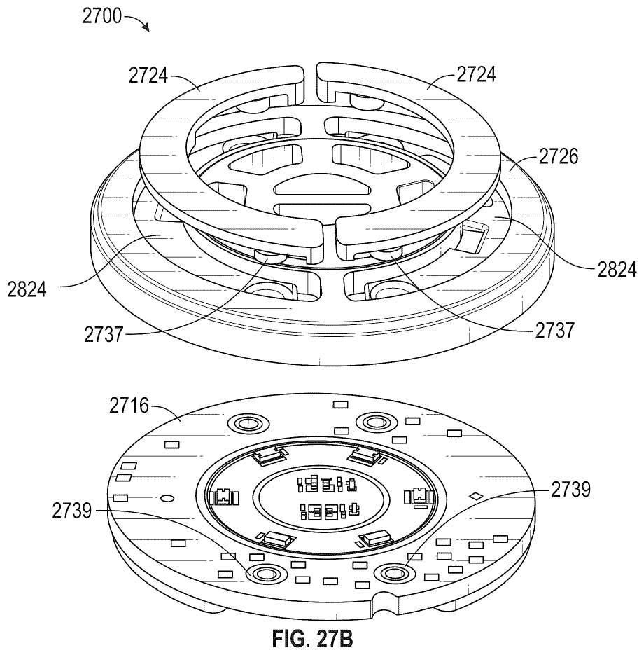

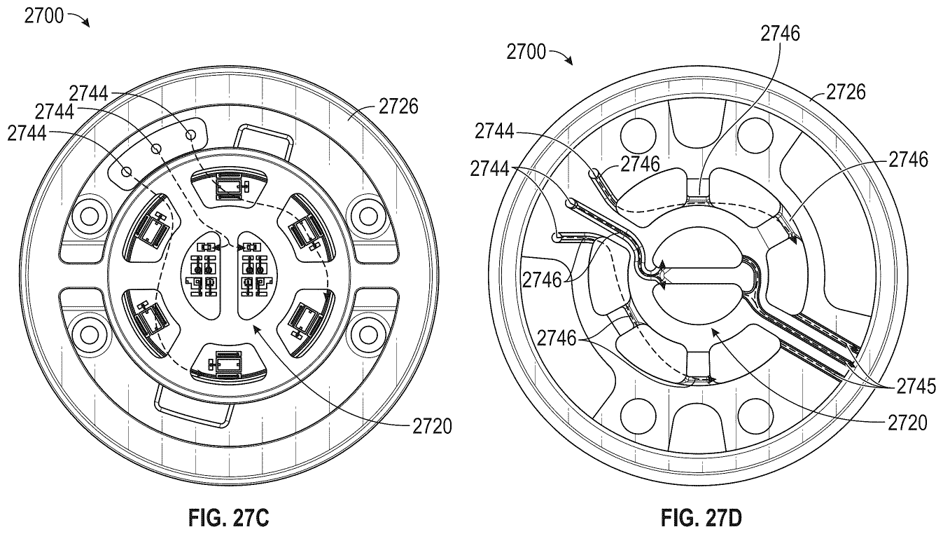

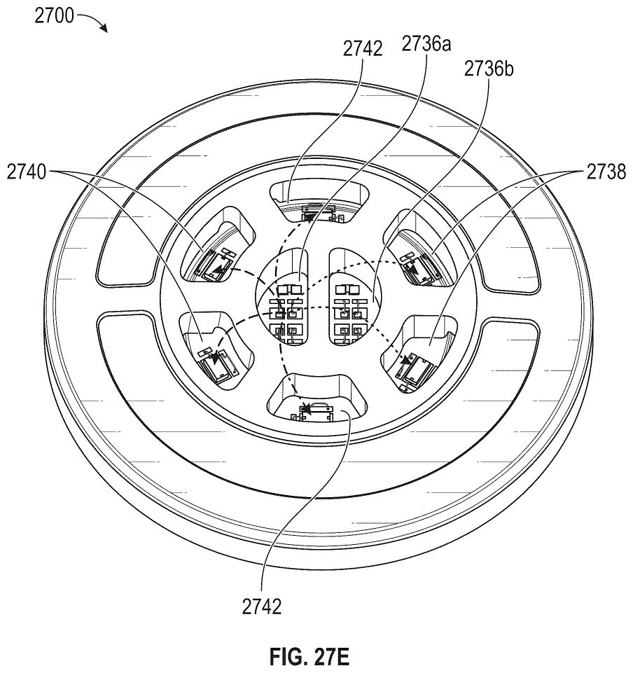

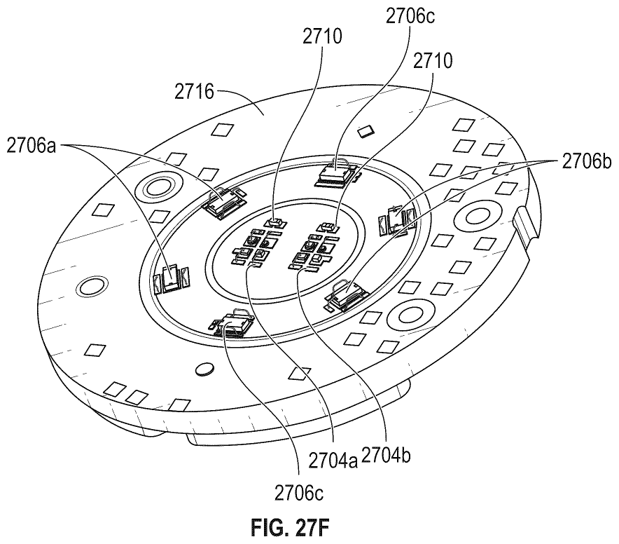

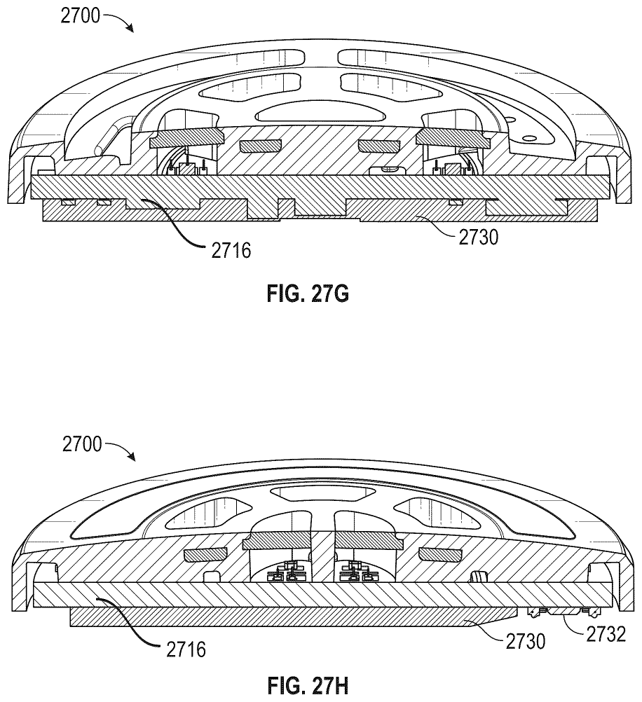

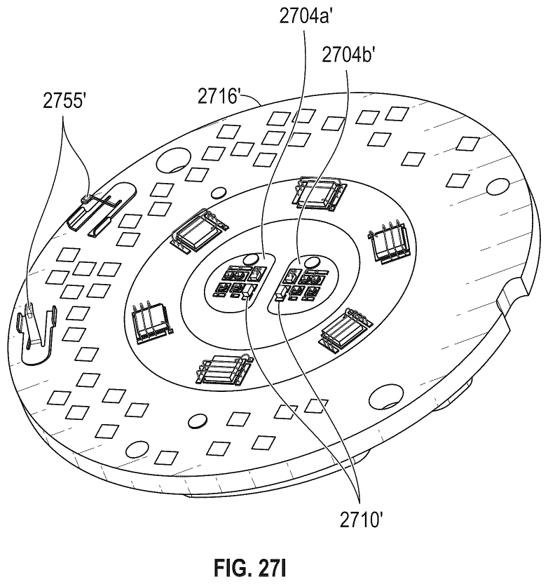

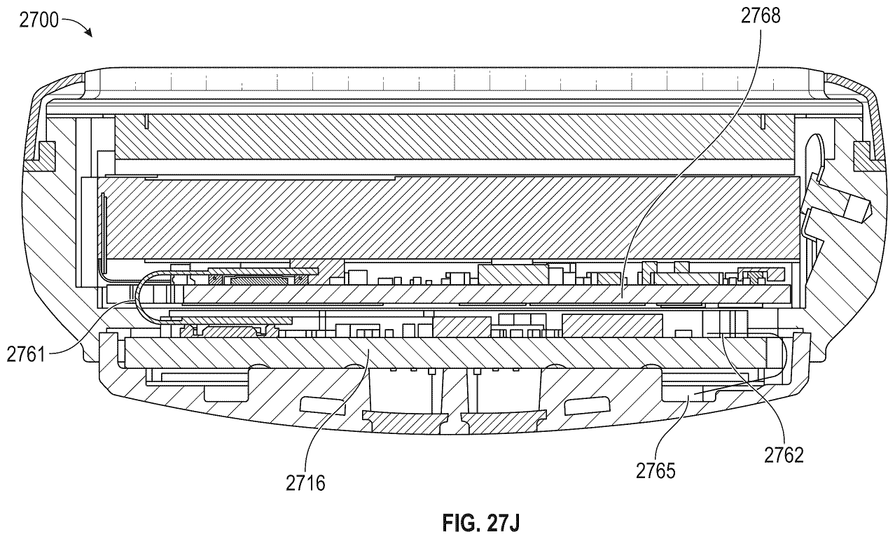

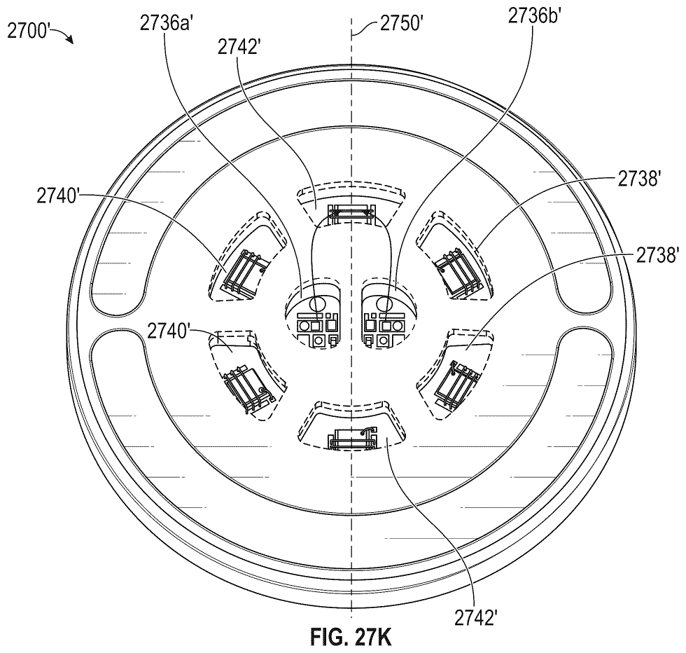

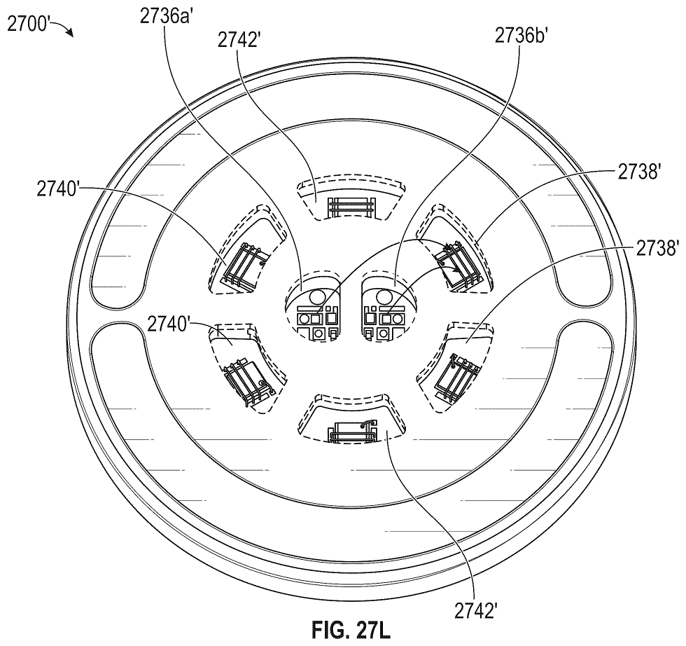

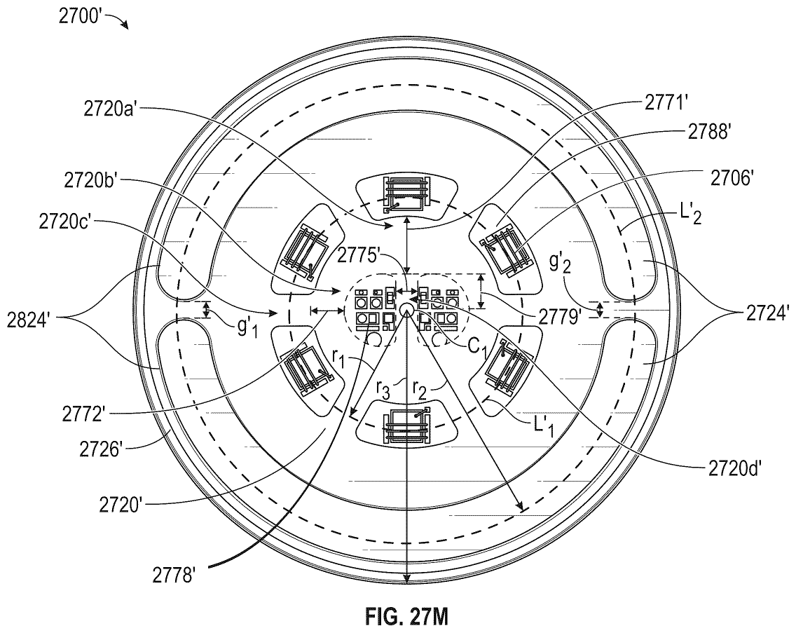

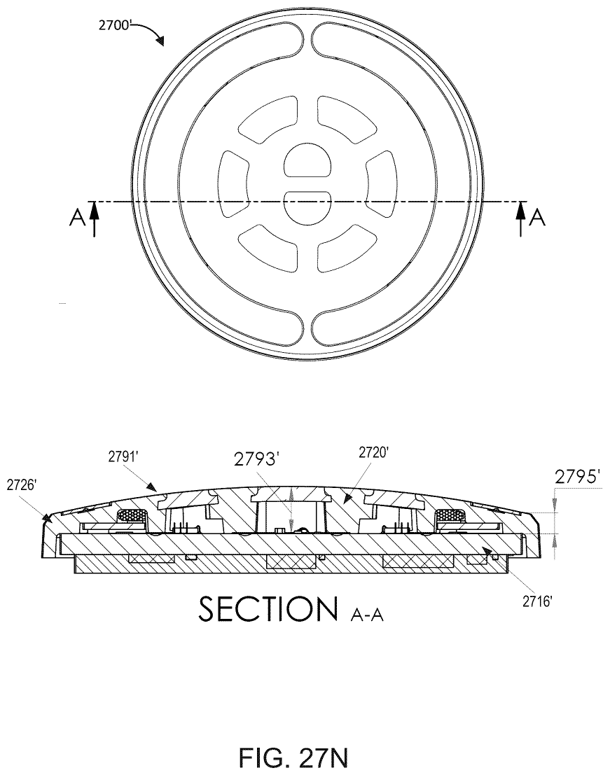

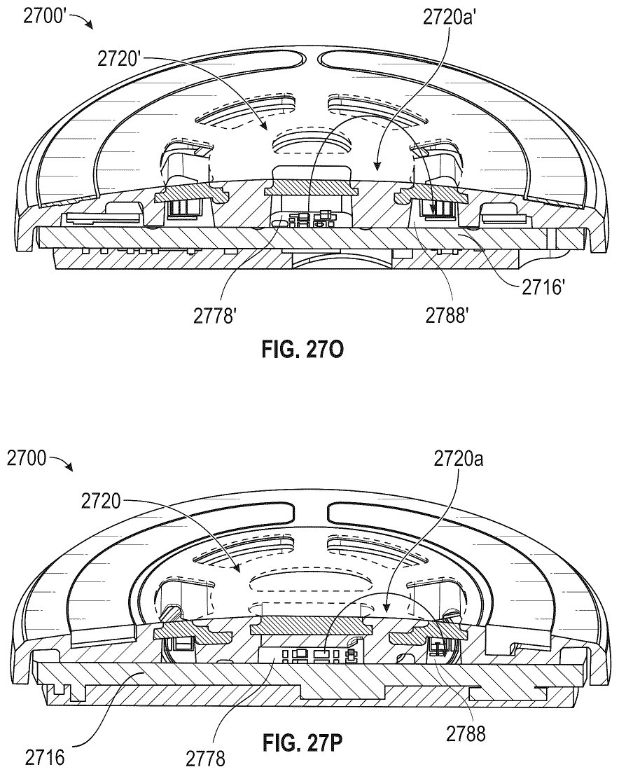

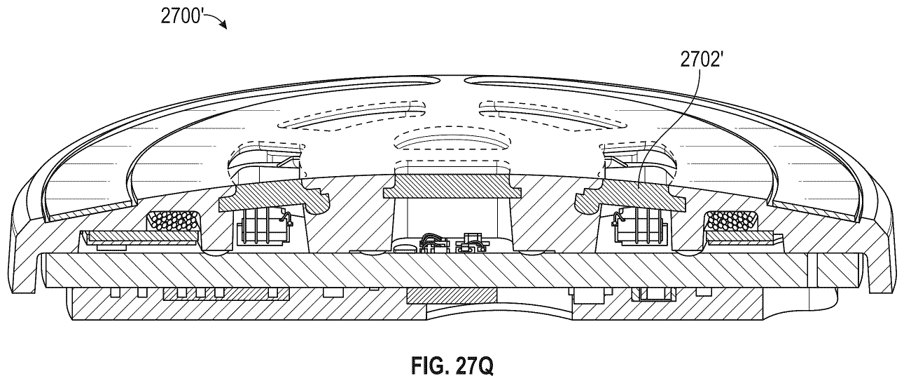

FIGS. 27A, 27B, and 27E illustrate an additional example aspect of an optional electrocardiogram (ECG) sensor. The electrocardiogram (ECG) sensor may include a plurality of electrodes 2724 configured to make contact with the wearer's skin. The plurality of ECG electrodes 2724 may be located on the sensor or module 2700 (such as shown in FIGS. 27A, 27B and 7E). As disclosed herein, the wearable device incorporating the module can include another ECG electrode 125 located on the housing of the wearable device configured to make contact with the wearer's skin.

FIG. 27B is an exploded perspective view of an example aspects of a sensor or module 2700. As shown in FIG. 27B, the opaque frame 2726 can include recesses (which may also be referred to as “indentations”) having the shape and size to accommodate the ECG electrodes 2724 or other components with a suitable shape and size. For example, in some implementations, frame 2726 includes recesses 2824. Recesses 2824 can be sized and/or shaped to receive ECG electrodes 2724. In some implementations, recesses 2824 have a depth (for example, measured from a plane of the frame 2726) that is substantially equal to a thickness of the ECG electrodes 2724. In some implementations, recesses 2824 have a size and/or shape that matches a size and/or shape of the ECG electrodes 2724. For example, in some implementations in which the ECG electrodes have a semi-annular shape (such as that illustrated in at least FIGS. 27A-27B), the recesses 2824 can have a semi-annular shape.

A front side of the electrodes 2724 can have one or more posts 2737 extending past openings in the opaque frame 2726 into corresponding openings on the substrate 2716. The posts 2737 of the electrodes 2724 can establish an electrical connection with the corresponding openings of the substrate 2716. A plurality of screws (or other types of fasteners) can extend into the corresponding openings of the substrate 2716 from the front side of the substrate 2716 to secure the electrodes 2724 to the sensor or module 2700 by threadedly mating or otherwise with the posts 2737. When a wearer puts the wearable device incorporating the sensor or module 2700 onto the wearer's wrist, the electrodes 2724 can make contact with the wearer's skin.

With continued reference to FIG. 27B, the substrate 2716 can include a printed circuit board (PCB). The substrate 2716 can include a conductive liquid adhesive 2739. The conductive liquid adhesive 2739 may be provided on the copper of the substrate 2716. The conductive liquid adhesive 2739 may facilitate conductive electrical connection between the electrodes 2724 and the substrate 2716.

With continued reference to FIG. 27B, one or more spring contacts (such as spring contacts 2755′ shown in FIG. 27I) may be located between the electrodes 2724 and the substrate 2716. The shape, size, and/or number of the spring contacts can vary. The spring contacts can establish an electrical connection between the electrodes 2724 and the substrate 2716. The spring contacts can be biased toward the electrodes 2724 to ensure a firm electrical connection between the spring contacts and the electrodes 2724 and the substrate 2716.

The physiological parameter measurement module 100 can include diffusing materials or encapsulant, which can include, for example, microspheres or glass microspheres. As described above, the encapsulant can eliminate air gaps between the surface of the light transmissive cover 102 and the emitters 104 and/or the detectors 106. The encapsulant can be included around the emitters 104 to more evenly spread the emitted light, which appears to be emitted from an entire emitter chamber rather than from a point source (that is, a single LED emitter) if the encapsulant is absent. The encapsulant can allow the emitted light to travel through a greater volume of the tissue at the tissue site. The diffusing material can act as a beam shaper that can homogenize the input light beam from the emitter, shape the output intensity profile of the received light, and define the way (for example, the shape or pattern) the emitted light is distributed to a tissue measurement site. Such diffuser materials can, for example, deliver substantially uniform illumination over a specified target area in an energy-efficient manner. According to the Beer-Lambert law, the amount of light absorbed by a substance is proportional to the concentration of the light-absorbing substance in the irradiated solution (for example, the arterial blood). Therefore, by irradiating a larger volume of tissue and/or by increasing the amount of detected light, a larger sample size of light attenuated by the wearer's tissue can be measured. The larger sample size provides a data set that can be more representative of the complete interaction of the emitted light as it passes through the patient's blood as compared to a smaller sample size.

The diffusing materials can be any suitable materials, for example, glass, ground glass, glass beads, opal glass, greyed glass, polytetrafluoroethylene, or a microlens-based, band-limited, engineered diffuser that can deliver efficient and uniform illumination UV-cured flow glass microspheres injected into one or more openings on the sensor or module 100 (for example, after the sensor or module 100 has been assembled). Examples of engineered diffusers can include molded plastics with specific shapes, patterns, and/or textures designed to diffuse the emitter light across the entirety of a tissue surface. The diffusing material can be made of ground glass, which spreads the emitted light with a Gausian intensity profile. The diffusing material can include glass beads. The diffusing material can be constructed so as to diffuse the emitted light in a Lambertian pattern. A Lambertian pattern is one in which the radiation intensity is substantially constant throughout the area of dispersion. One such diffusing material can be made from opal glass. Opal glass is similar to ground glass, but has one surface coated with a milky white coating to diffuse light evenly. The diffusing material can be capable of distributing the emitted light on the surface of a plane (for example, the surface of the tissue measurement site) in a predefined geometry (for example, a rectangle, square, circle, or otherwise), and with a substantially uniform intensity profile and energy distribution. The efficiency, or the amount of light transmitted by the diffusing material, can be greater than 70% of the light emitted by the emitter. The efficiency can be greater than 90% of the emitted light. Additional examples of the diffusing material are described in U.S. Pat. No. 10,448,871, the entirety of which is hereby incorporated herein by reference and should be considered part of the disclosure.

Additionally or alternatively, the physiological parameter measurement module 100 can include encapsulant or light diffusing materials in the detector chambers to more evenly spread the reflected light to so as to increase the amount of the reflected light reaching the detectors. The module can include light diffusing materials positioned around the detectors to scatter and/or deflect the reflected light so that more reflected light can be detected by the detectors. For example, the reflected light can keep bouncing off the diffusing materials until the reflected light reaches the detector. Accordingly, the light detecting surface area in the module can be greater than the surface area of the detectors. Having the light diffusing materials can reduce the power needed to drive the LEDs of the emitters and/or the number of detectors at a particular location of the module, which can reduce the power consumption of the module.

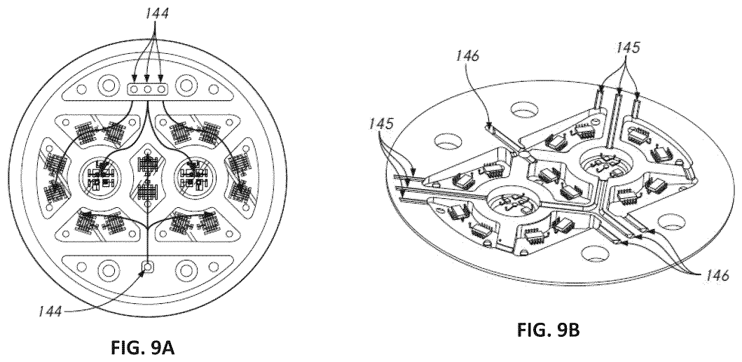

As shown in FIG. 9A, the opaque frame 126 of the sensor or module 100 can include a plurality of light diffusing material(s) (or encapsulant) fill holes 144. Light diffusing material(s) or encapsulant (for example, a flow of glass microspheres) can be injected into the plurality of chambers via the fill holes 144, and be directed to the respective emitter or detector chambers as illustrated by the arrows in FIG. 9A along a plurality of the fill channels 146 (see FIG. 9B) which are interconnected with the fill holes 144. The fill channels 146 can be located at a side of the opaque frame 126 facing away from the tissue of the wearer. As shown in FIG. 9B, the side of the opaque frame 126 facing away from the tissue of the wearer can further include a plurality of air vent channels 145. Air can escape into the vent channels 145 as the diffusing material solution or encapsulant is injected into the respective chambers via the fill holes 144, making it easier for the injected solution to flow into the respective chamber. As shown in FIG. 9B, the module 100 may not have air vent channels or fill channels between emitter and detector chambers to avoid light piping along such a channel. The encapsulant can be UV-cured after being injected into the respective chambers.

The opaque frame 126 may be configured such that the fill holes 144 and channels 146 allow the light diffusing materials to fill only the emitter chambers, or only the detector chambers, or both the emitter and detector chambers. Optionally, in addition or alternative to the light diffusing materials, the detector chamber can include light transmissive lens(es) or covers on the surface of the PCB that is not occupied by the detectors. The light transmissive lens(es) or covers may be polycarbonate. The light transmissive lens(es) or covers inside the detector chamber can help in focusing the reflected light onto the detectors inside the detector chamber.

FIGS. 27C and 27D illustrate another example opaque frame 2726 and light barrier construct 2720 of a sensor or module 2700. The opaque frame 2726 and/or light barrier construct 2720 can include a plurality of light diffusing material(s) (or encapsulant) fill holes 2744. Light diffusing material(s) or encapsulant (for example, a flow of glass microspheres) can be injected into the plurality of chambers via the fill holes 2744, and be directed to the respective emitter or detector chambers as illustrated by the red arrows in FIGS. 27C and 27D along a plurality of the fill channels 2746 (see FIG. 27D) which are interconnected with the fill holes 2744. The fill channels 2746 can be located at a side of the opaque frame 2726 facing away from the tissue of the wearer. The fill channels 2746 may travel through portions of the opaque frame 2726 and/or portions of the light barrier construct 2720. As shown in FIG. 27D, the side of the opaque frame 2726 facing away from the tissue of the wearer can further include a plurality of air vent channels 2745. Air can escape into the vent channels 2745 as the diffusing material solution or encapsulant is injected into the respective chambers via the fill holes 2744, making it easier for the injected solution to flow into the respective chamber. As shown in FIG. 27D, each of the fill channels 2746 and/or the vent channels 2745 may connect more than one emitter chamber or more than one detector chamber. However, the module 2700 may not have air vent channels or fill channels extending between emitter and detector chambers to avoid light piping along such a channel. The encapsulant can be UV-cured after being injected into the respective chambers.

The opaque frame 2726 may be configured such that the fill holes 2744 and channels 2746 allow the light diffusing materials to fill only the emitter chambers, or only the detector chambers, or both the emitter and detector chambers.

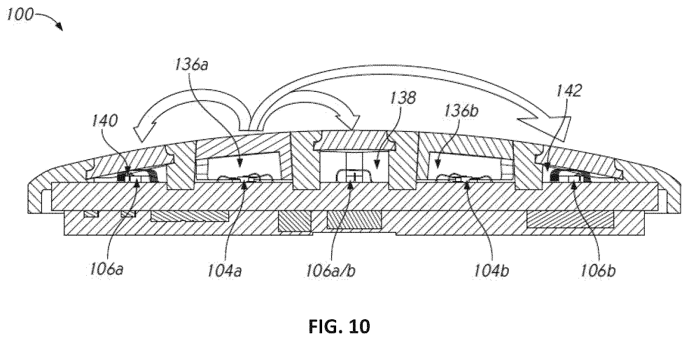

In FIG. 10, a cross-sectional view of the sensor or module 100 illustrates some of the emitter and detector chambers. The chambers illustrated in FIG. 10 include a first emitter chamber 136a enclosing a first emitter group 104a, a second emitter chamber 136b enclosing a second emitter group 104b, a first detector chamber 140 enclosing one of first groups of detectors 106a that surround the first emitter group 104a, a second detector chamber 142 enclosing one of second groups of detectors 106b that surround the second emitter group 104b, and a third detector chamber 138 enclosing one of shared groups of detectors 106a/b that surround both the first and second emitter groups 104a, 104b on opposite sides of the third detector chamber 138.

As shown in FIG. 10, light from the first emitter group 104a can travel a shorter path, as indicated by the shorter arrows, to the first group of detectors 106a or the shared group of detectors 106a/b; and light from the first emitter group 104a can travel a longer path, as indicated by the longer arrows, to the second group of detectors 106b. The reverse is true for light from the second emitter group 104b, which can travel a shorter path to the second group of detectors 106b or the shared group of detectors 106a/b and a longer path to the first group of detectors 106a. As described herein, the different groups of emitters 104a, 104b and/or detectors 106a, 106b, 106a/b can be run independently and/or simultaneously. Signals outputted by the different groups of detectors 106a, 106b, 106a/b based on light emitted from the first emitter group 104a and/or the second emitter group 104b can provide different information due to the different light paths, which can travel through different areas of the tissue. The longer path penetrates deeper into the tissue and through a greater volume of the tissue to reach the “far” groups of detectors than the shorter path, which penetrates less deep into the tissue and travels through a smaller volume of tissue to reach the “near” group of detectors. The different information can be separated and/or combined to calculate a plurality of physiological parameters of the wearer of the sensor or module 100, for example, an indication of the wearer's hydration status, which will be described in greater detail below.

FIG. 27E is a front perspective view of an example aspect of a sensor or module 2700 including another example arrangement of emitter and detector chambers. The emitter and detector chambers may comprise one or more light blocks. The chambers illustrated in FIG. 27E include a first emitter chamber 2736a enclosing a first emitter group, a second emitter chamber 2736b enclosing a second emitter group, a first group of detector chambers 2740, a second group of detector chambers 2742, and a third group of detector chambers 2738. Each detector chamber can enclose one detector. The first and second emitter chambers 2736a, 2736b can be adjacent to each other. The first, second and third groups of detector chambers 2740, 2742, 2738 can extend around the first and second emitter chambers 2736a, 2736b.

As shown in FIG. 27E, light from the first and second emitter groups in the first and second emitter chambers, respectively, can emit light that travel paths of different lengths for example to different detectors. Light from the first emitter group can travel a shorter path, as indicated by the shorter arrows, to the first group of detector chambers 2740; and light from the first emitter group can travel an intermediate path, as indicated by the intermediate arrows, to the second group of detector chambers 2742; and light from the first emitter group can travel a longer path, as indicated by the longer arrows, to the third group of detector chambers 2738. The reverse is true for light from the second emitter group, which can travel a shorter path to the third group of detector chambers 2738, and an intermediate path to the second group of detector chambers 2742, and a longer path to the first group of detector chambers 2740.

As described herein, the different emitters can be run independently and/or simultaneously. For example, the emitters can be selectively activated (e.g., modulated) so that only one emitter (or subset of emitters) is emitting light at a given time. For example, in aspects wherein the first emitter group comprises four emitters, each of the four emitters of the first emitter group may be activated for a quarter cycle (e.g., a different quarter cycle than the other emitters) and off for the remaining three-quarters cycle. For example, a first emitter of the first emitter group may be activated to emit light during only a first quarter cycle, a second emitter of the first emitter group may be activated to emit light during only a second quarter cycle, a third emitter of the first emitter group may be activated to emit light during only a third quarter cycle and a fourth emitter of the first emitter group may be activated to emit light during only a fourth quarter cycle. The emitters of the second emitter group may operate in a similar manner as described.

As another example, in aspects wherein the first emitter group comprises four emitters, each of the four emitters of the first emitter group may be activated for an eighth of a cycle (e.g., a different eighth of the cycle than the other emitters) and off for the remaining seven-eighths cycle. An eighth of a cycle wherein no emitter is activated may occur between each of the cycles wherein an emitter is activated. For example, a first emitter of the first emitter group may be activated to emit light during only a first quarter cycle, a second emitter of the first emitter group may be activated to emit light during only a third quarter cycle, a third emitter of the first emitter group may be activated to emit light during only a fifth quarter cycle and a fourth emitter of the first emitter group may be activated to emit light during only a seventh quarter cycle. The emitters of the second emitter group may operate in a similar manner as described.

The above examples are not meant to be limiting. Alternative activation sequences for the emitters may be used to provide a time-multiplexed signal. In some aspects, the emitters can be selectively activated (e.g., modulated) so that two or more emitters are emitting light at a given time (e.g., during the same cycle or during overlapping cycles), for example in a manner similar to the examples given above.

The emitters may be modulated within an emitter group (e.g., first emitter group and second emitter group) or all of the emitters of the wearable device 10 may be modulated according to a single activation sequence. For example, the emitters of the first group may be modulated according to one activation sequence and the emitters of the second group may be modulated according to a second activation sequence. Alternatively, the emitters of the first and second emitters groups can all be modulated according to a single activation sequence.

In some aspects, the detectors may operate independently from and/or simultaneously with each of the other detectors. For example, each of the detectors may provide an individual signal to the module processor 108.

Signals outputted by the different detectors of the different detector chambers 2740, 2742, 2738 based on light emitted from the first emitter group and/or the second emitter group can provide different information due to the different light paths, which can travel through different areas of the tissue. The longer path penetrates deeper into the tissue and through a greater volume of the tissue to reach the detectors of the “far” group of detector chambers than the intermediate and shorter paths. The shorter path penetrates less deep into the tissue and travels through a smaller volume of tissue to reach the detectors of the “near” group of detector chambers than the intermediate and longer paths. The different information can be separated and/or combined to calculate a plurality of physiological parameters of the wearer of the sensor or module 2700.

For convenience, the terms “proximal” and “distal” are used herein to describe structures relative to the first emitter group or the second emitter group. For example, a detector may be proximal or distal to the first emitter group and may be proximal or distal to the second emitter group. The term “distal” refers to one or more detectors that are farther away from an emitter group than at least some of the other detectors. The term “proximal” refers to one or more detectors that are closer to an emitter group than at least some of the other detectors. The term “intermediate detector” refers to detectors that are closer to an emitter group than distal detectors and farther from an emitter group than proximal detectors. The term “proximal detector” may be used interchangeably with “near detector” and the term “distal detector” may be used interchangeably with “far detector”.

A single detector may be both a proximal to one detector and distal to another detector. For example, a detector may be a proximal detector relative to the first emitter group and may be a distal detector relative to the second emitter group.

FIG. 11A illustrates schematically an example wearable device 10 disclosed herein. As described above, the device processor 14 can be connected to the module sensor 108 of the physiological parameter measurement module 100, which includes the emitters, the detectors, the thermistors, and other sensors disclosed herein. The electrical connection between the device processor 14 and the sensor or module processor 108 can be establish optionally via a flex connector 32. The sensor or module processor 108 can be coupled to the ECG electrodes 124, 125, optionally via an ECG flex connector 123.

The device processor 14 can be connected to a display 12, which can include the display screen and touch input from the wearer. The device processor 14 can include a battery 16, and optionally one or more wireless charging coils 17 to enable wireless charging of the battery 16. The device processor 14 can be connected to an antenna 19 for extending signals transmitted wirelessly, for example, to an external device as described with reference to FIG. 2. The device processor 14 can include connection to a first user interface (UI 1) 13a and a second user interface (UI 2) 13b on the device 10 to receive input from the wearer. As shown in FIG. 1D, example first and second user interface 13a, 13b can be in the form of buttons 13. Additionally or alternatively, the device 10 can include a microphone. The device 10 can receive user inputs via the user interfaces, which can be the buttons, the microphone, and/or the touchscreen. The user inputs can command the device 10 to turn on and/or off certain measurements, and/or to control externally connected devices, such as an insulin pump, a therapeutics delivery device, or otherwise. The device processor 14 can be connected to a user feedback output 15 to provide feedback to the wearer, for example, in the form of vibration, an audio signal, and/or otherwise. The device processor 14 can optionally be connected to an accelerometer and/or a gyroscope 42 located on the device 10 that is different from the accelerometer 114 and gyroscope 112 on the physiological parameter measurement module 100. The accelerometer and/or gyroscope 42 can measure position and/or orientation of the wearer for non-physiological parameter measurement functions, for example, for sensing that the wearer has woken up, rotating the display 12, and/or the like.

FIG. 11B illustrates example components of the device processor 14 PCB board. As shown in FIG. 11B, the device processor 14 can include a Bluetooth co-processor 1400 and a system processor 1402. The system processor 1402 can run the peripheral functions of the device 10, receive user (that is, the wearer) input and communicate to the sensor or module processor 108. The Bluetooth co-processor 1400 can focus on managing Bluetooth communication so as to allow the system processor 1402 to focus on the high memory utilization tasks, such as managing the display screen 12. The Bluetooth co-processor 1400 can be activated when there is incoming and/or outgoing Bluetooth communication. Alternatively, the Bluetooth co-processor 1400 can be replaced by a different wireless co-processor configured to manage wireless communication using a different wireless communication protocol.

FIG. 11C illustrates example components of the module processor PCB board 116. As shown in FIG. 11C, the sensor or module processor 108 can include a calculation processor 1080 and a system processor 1082. The calculation processor 1080 can manage host communication with the device processor 14 via a host connector 1084. The calculation processor 1080 can perform algorithm computations to calculate the physiological parameters based on the signals received from the ECG electrodes 124/125 and the optical sensor including the emitters 104, the detectors 106, and the temperature sensors 110, and optionally from other sensors in communication with the sensor or module processor 108. The calculation processor 1080 can have relatively large memory suitable for running algorithm computations. The system processor 1082 can be in communication with a power management integrated circuit (PMIC) 1090. The system processor 1082 can run the physical system of the sensor or module 100 (for example, including turning on and off the emitter LEDs, changing gain, setting current, reading the accelerometer 114 and/or the gyroscope 112, and the like) and decimate data to a lower sampling rate. The system processor 1082 can focus on data processing, taking measurements and diagnostics, and basic functions of the sensor or module processor 108. The system processor 1082 can allow the calculation processor 1082 to sleep (being inactive) most of the time, and only wake up when there is enough measurement data to perform calculations.

FIG. 11D illustrates an example front-end analog signal conditioning circuitry 1088 of the module PCB 116 shown in FIG. 11C. The entire front end circuitry 1088 can be located on a single application-specific integrated circuit (ASIC).

The front-end circuitry 1088 can include a transimpedance amplifier 1092 configured to receive analog signals from the optical sensor including the emitters 104, the detectors 106, and the temperature sensors 110, which can be preprocessed (for example, via a low pass filter 1094 and a high pass filter 1096) before being sent to an analog-digital converter 1098. The analog-digital converter 1098 can output a digital signal based on the analog signals from the optical sensor including the emitters 104, the detectors 106, and the temperature sensors 110 to the system processor 1082 and the calculation processor 1080. The front end circuitry 1088 can include a detector cathode switch matrix 1083 configured to activate the cathode of the detectors that are selected to be activated. The matrix 1082 can be further configured to deactivate (for example, by short-circuiting) anodes of the detectors that are selected to be deactivated in configurations in which the detectors share a common cathode and have different cathodes.

The front-end circuitry 1088 can include an ECG amplifier 1091 configured to receive analog signals from the ECG electrodes 124/125, which can output the amplified analog signals to the analog-digital converter 1096. The amplified analog signals can include an ECG differential between the positive and negative electrodes. The analog-digital converter 1098 can output a digital signal based on the analog signals from the ECG electrodes 124/125 to the system processor 1082 and the calculation processor 1080.

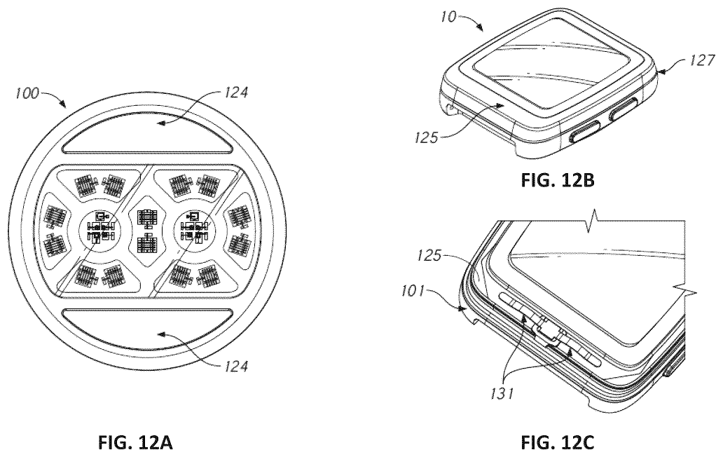

The ECG electrodes 124 can include a negative electrode, a positive electrode, and a reference electrode. As shown in FIG. 12A, the two electrodes 124 located on the sensor or module 100 can act as a reference electrode and a negative (or positive) electrode respectively. As shown in FIGS. 12B and 12C, a portion of the device housing 101 that surrounds the display screen 12 can function as another ECG electrode 125. An electrically insulating material 127 can separate the ECG electrode 125 from the remainder of the housing 101 so that an electrical current between the ECG electrode 125 and the ECG electrodes 124 would travel through the wearer's body. When the wearer wants to make a measurement using the ECG sensor that includes the ECG electrodes 124, 125, the wearer can press on or touch the electrode 125 using the wearer's finger or another part of the wearer's body such that the wearer's skin makes contact with the electrode 125.

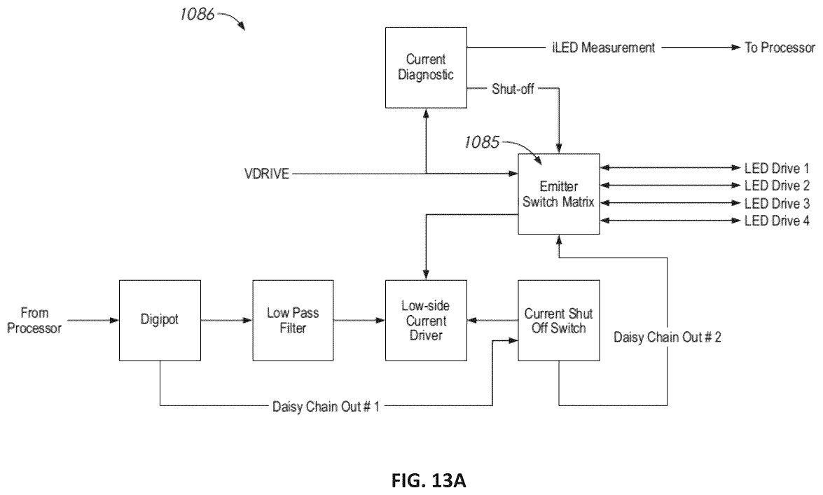

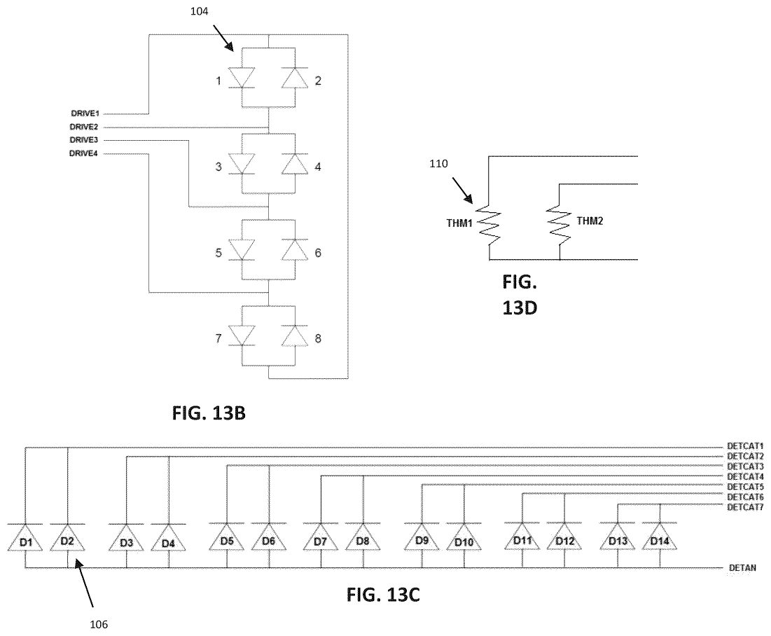

In the illustrated examples, the ECG electrode 125 can be positive (or negative if one of the electrodes 124 servers as a positive electrode) electrode. As shown in FIG. 12C, the electrode 125 is illustrated as being transparent to show one or more spring contacts 131 located underneath the electrode 125. The shape, size, and/or number of the spring contacts 131 can vary from the example shown in FIG. 12C. The spring contacts 131 can establish an electrical connection between the electrode 125 and the electrode 125 and the sensor or module processor 108 of the sensor or module 100. For example, the spring contacts 131 can establish an electrical connection between the electrode 125 and the connector 132. The spring contacts 131 can be biased toward the electrode 125 to ensure a firm electrical connection between the spring contacts 131 and the electrode 125. Readings from the electrodes 124, 125 can allow the sensor or module processor 108 to obtain the wearer's ECG signal and optionally to make physiological measurements based on the obtained ECG, for example, the heart rate, the respiratory rate, and/or otherwise. The sensor or module processor 108 can communicate the ECG signals and/or ECG-related measurements to the wearable device processor 14. The wearer's ECG waveform and/or the measurements made from the ECG can be displayed on the display screen 12. FIG. 13A illustrates an example LED driver circuitry 1086 of the module PCB 116 shown in FIG. 11C. The entire LED driver circuitry 1086 can be located on the single ASIC with the front end circuitry 1088. As described above, the system processor 1802 can output a control signal to turn on and off the emitter LEDs. As shown in FIG. 13A, the LED driver circuitry 1086 can include an emitter switch matrix 1085 configured to drive any of the emitters (or emitter groups) that are selected to be turned on or turn off any of the emitters (or emitter groups) that are selected to be turned off.

FIG. 13B illustrates an example emitter circuitry including eight different emitter LEDs 104. The number of LEDs may vary and can be less than or greater than eight. The emitters of the physiological parameter measurement module can be configured to emit a plurality of (for example, three, four, or more) wavelengths. Each of the emitters can be configured to emit light of a different wavelength than the other emitters. Alternatively, one or more of the emitters can emit light of more than one wavelength. In the illustrated example, the emitter circuitry can include four drivers to drive the eight emitter LEDs. Alternatively, the module can include more than four LEDs per emitter group. Each LED Drive can drive an LED to emit light of a different wavelength. The device or the module can grant access of some of the LEDs to a third party device, for example, for measurement purposes. The LED drivers can selectively drive some but not all the LEDs.

The emitters can be configured to emit light of a first wavelength providing an intensity signal that can act as a reference signal. The first wavelength can be more absorbent by the human body than light of other wavelengths emitted by the emitters. The reference signal can be stronger and less likely to be affected by noise than the signals from other wavelengths emitted by the emitters. The reference signal can be used by the physiological parameter measurement sensor or module processor to extract information from the other signals, for example, information relevant to and/or indicative of the pulsing rate, harmonics, or otherwise. The physiological parameter measurement sensor or module processor can focus the analysis on the extracted information for calculating physiological parameters of the wearer. Including the reference signal can reduce power consumption and saving the battery life of the device. The first wavelength can be from about 525 nm to about 650 nm, or from about 580 nm to about 585 nm, or from about 645 nm to about 650 nm, or about 525 nm, or about 580 nm, or about 645 nm. The light providing the reference signal can have an orange or yellow color. Alternatively, the light providing the reference signal can have a green color.

The emitters can be configured to emit light having a second wavelength having a red color. The second wavelength can be from about 620 nm to about 660 nm. Light of the second wavelength can be more sensitive to changes in oxygen saturation (SpO2) than light of other wavelengths emitted by the emitters. The second wavelength is preferably closer to 620 nm (for example, about 625 nm), which results in greater absorption by the body tissue of the wearer, and therefore a stronger signal and/or a steeper curve in the signal, than a wavelength that is closer to 660 nm. The physiological parameter measurement sensor or module processor 108 can extract information such as the pleth waveform from signals of the second wavelength.

The emitters can be configured to emit light having a third wavelength of about 900 nm to about 910 nm, or about 905 nm, or about 907 nm. The third wavelength can be in the infrared range. The sensor or module processor can use the third wavelength as a normalizing wavelength when calculating ratios of the intensity signals of the other wavelengths, for example, a ratio of the intensity signals of the second wavelength (red) to the third wavelength (infrared).

Additionally or optionally, the emitters can be configured to emit light having a fourth wavelength that is more sensitive to changes in water than the rest of the emitted wavelengths. The fourth wavelength can be in the infrared range and about 970 nm. The physiological parameter measurement sensor or module processor can determine physiological parameters such as a hydration status of the wearer based at least in part on a comparison of the intensity signals of the fourth wavelength and a different wavelength detected by certain detectors. The detectors used for hydration monitoring can be located a predetermined distance away from the emitters (that is, being a “far” detector disclosed herein) so that light travels through a certain depth of the tissue before being detected by those detectors.

The emitters in the physiological parameter measurement sensor or module can be placed in two emitter groups. Each emitter group can include four emitter LEDs configured to emit the first, second, third, and fourth wavelengths described above. The emitters in the same emitter group can be located in the same emitter chamber disclosed herein. Each of the four drivers are configured to drive the emitters to emit one of the four wavelengths described above.

In some aspects, drivers may drive the emitters at varying intensities. The intensity at which the drivers drive the emitters may affect the amount of light that is outputted (e.g., lumens), the strength of the light signal that is outputted, and/or the distance that the outputted light travels. The drivers may drive the emitters at varying intensities according to modeling, logic and/or algorithms. The logic and/or algorithms may be based, at least in part, on various inputs. The inputs may include historical data, the amount of light that is attenuated, for example as the light penetrates and travels through the tissue of the wearer, or the amount of blood with which the light is interacting, or the type of blood (e.g., venous, arterial) or type of blood vessel (e.g., capillary, arteriole) with which the light is interacting and/or the heat being generated by the emitters. For example, the drivers may increase the intensity at which they drive the emitters based upon a determination that too much light is being attenuated in the tissue or that the light is not interacting with enough blood. As another example, the drivers may decrease the intensity at which they drive the emitters based upon a determination that the emitters have exceeded a threshold temperature. The threshold temperature may be a temperature which may be uncomfortable for human skin.

In some aspects, each of the drivers may be capable of driving a corresponding emitter at various intensities independently of the other drivers. In some aspects, each of the drivers may drive a corresponding emitter at various intensities in unison with each of the other drivers.

Additionally, various LEDs may be used in various aspects. For example, certain LEDs may be used which are capable of outputting more light with the same amount of power as other LEDs. These LEDs may be more expensive. In some aspects, less expensive LEDs may be used. In some aspects, a combination of various types of LEDs may be used.

FIG. 13C illustrates an example detector circuitry including fourteen detectors 106. The total number of detectors on a module can vary. The fourteen detectors can form seven detector groups, each group including two detectors. The number of detectors in each group may vary. Detectors of the same detector group can be located in the same detector chamber disclosed herein. Each detector group can output one signal, which can be a combined signal of the two detectors in the same group. As shown in FIG. 13C, the detectors can share a common anode but have seven different cathodes, corresponding to the seven detector groups.

FIG. 13D illustrates an example thermistor circuitry. In the illustrated example, the physiological parameter measurement module can include two thermistors 110. The two thermistors can be located in the two emitter chambers near the two emitter groups respectively.

### Example Signal Processing of the Physiological Parameter Measurement Module

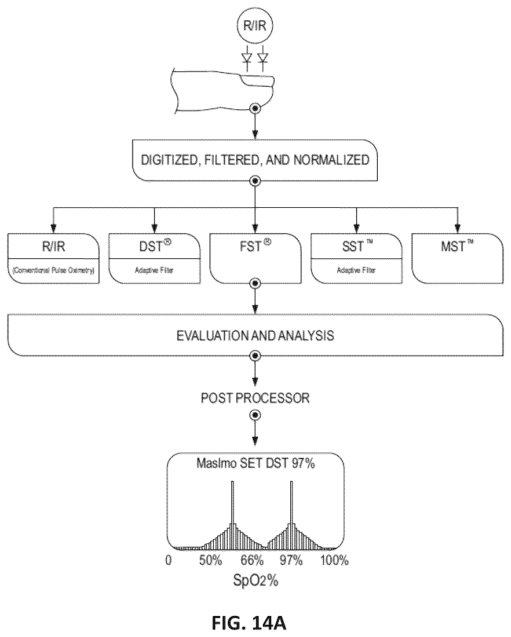

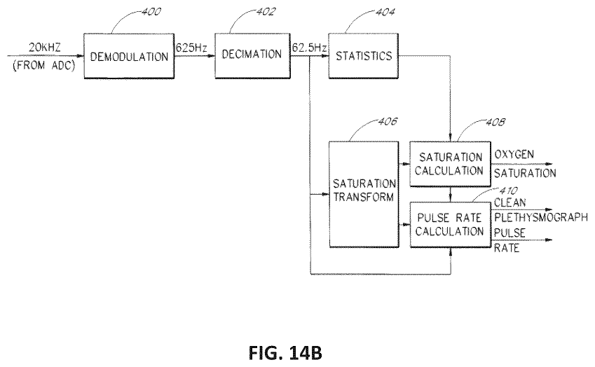

FIGS. 14A and 14B depict functional block diagrams of the operations of a conventional pulse oximeter carried out by the digital signal processing system. The signal processing functions described below are carried out by a digital signal processor (DSP) with a microcontroller providing system management. As shown in FIG. 14A, an analog signal from the detector(s) of the conventional pulse oximeter is digitized, filtered and normalized, and further processed using conventional pulse oximetry signal processing algorithms. Parallel signal processing engines-DST®, FST®, SST™, and MST™ are used to separate the arterial signal from sources of noise (including the venous signal) to measure SpO2 and pulse rate accurately, even during motion. FIG. 14B depicts a generalized functional block diagram for the operations performed on the 20 Khz sample data entering the digital signal processing system from an analog to digital converter (ADC). As illustrated in FIG. 14B, the DSP first performs a demodulation, as represented in a demodulation module 400. The processor performs decimation, as represented in a decimation module 402 on the resulting data from the demodulation. The processor calculates certain statistics, as represented in a statistics module 404, and performs a saturation transform, as represented in a saturation transform module 406, on the data resulting from the decimation operation. The processor forwards data subjected to the statistics operations and the data subjected to the saturation transform operations to saturation operations, as represented by a saturation calculation module 408 to output an oxygen saturation measurement and pulse rate operations, as represented in a pulse rate calculation module 410 to output a pulse rate value.

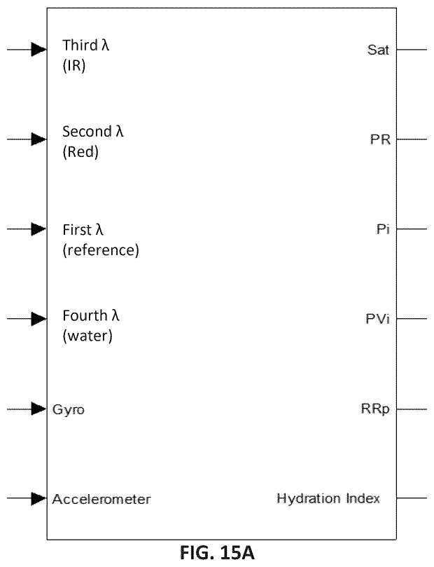

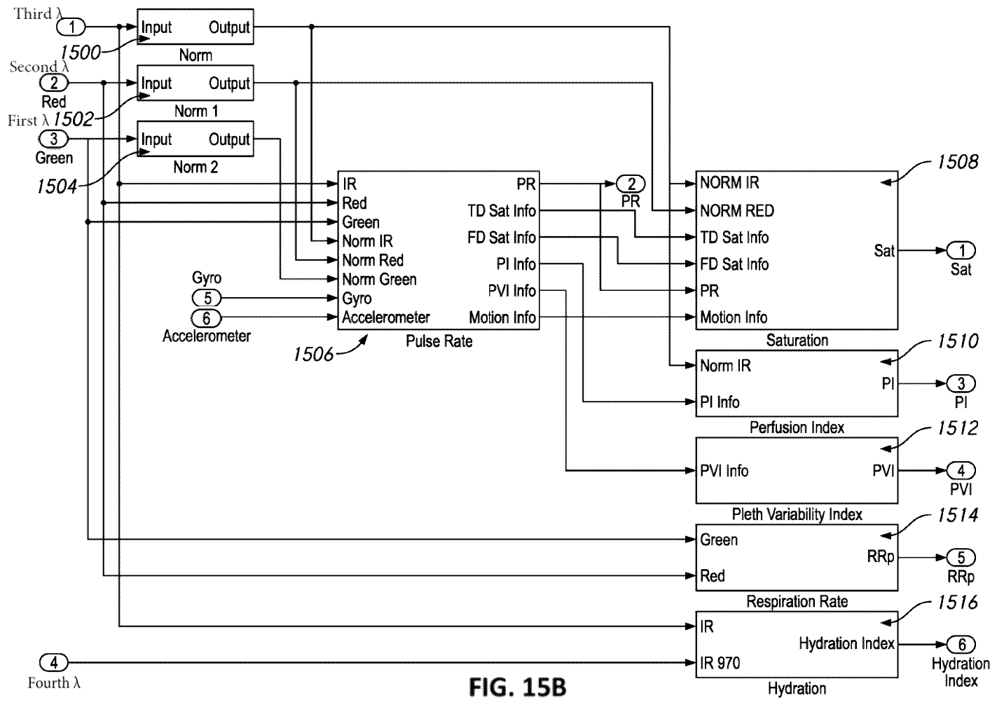

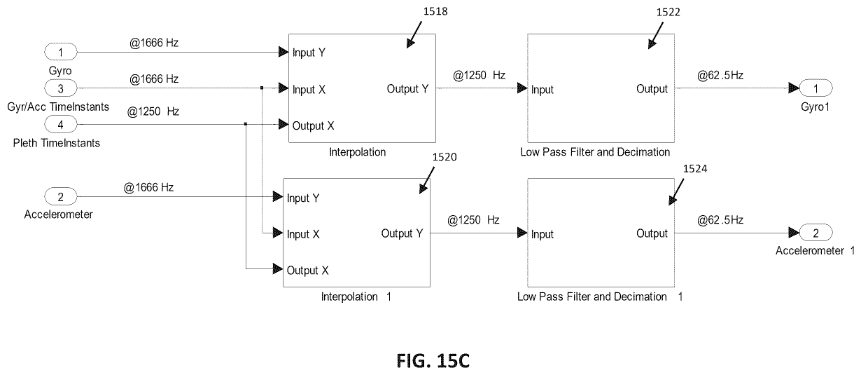

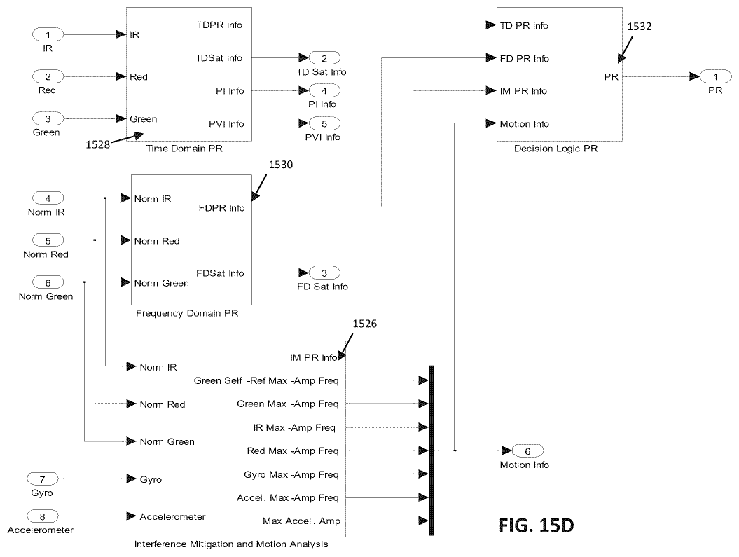

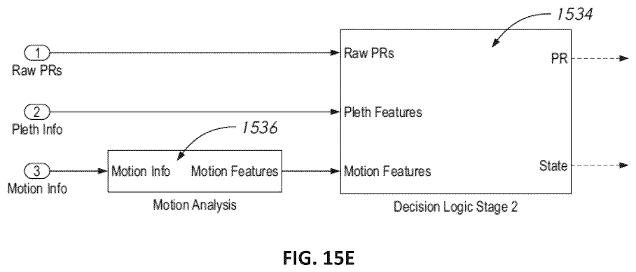

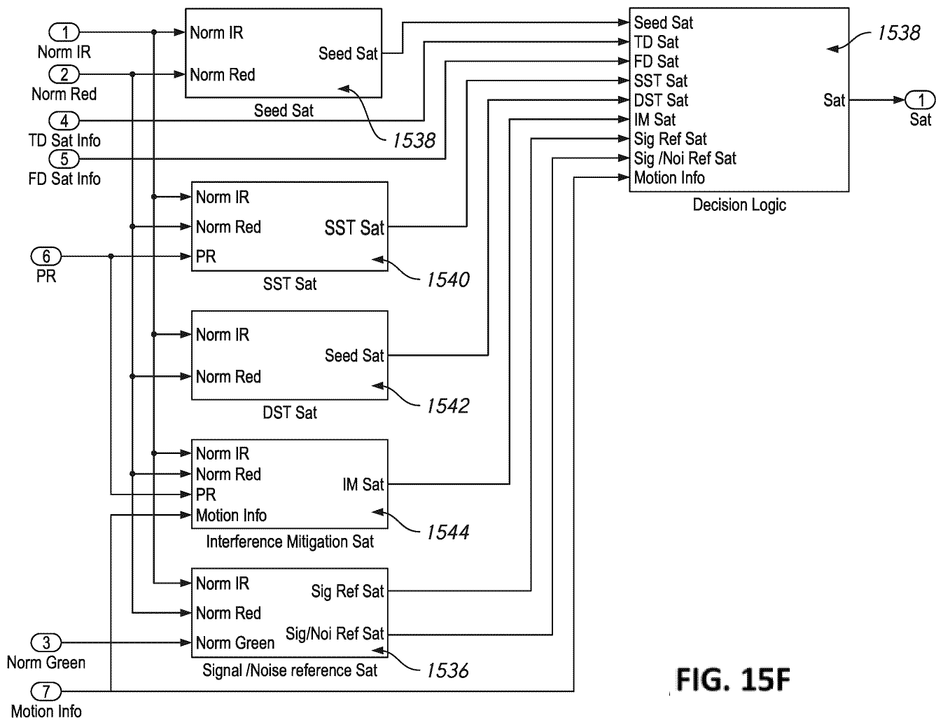

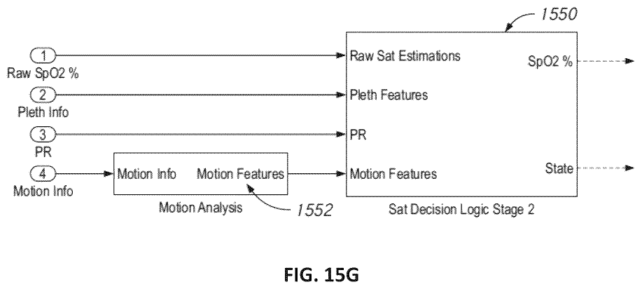

FIGS. 15A-15G illustrate example signal processing of the physiological parameter measurement sensor or module disclosed herein. As shown in FIG. 15A, the sensor or module processor can receive intensity signals from the detectors in response to detected reflected light of the first (reference signal or signal of green or yellow light), second (signal of red light), third (signal of infrared light), and fourth (signal of infrared light with a wavelength of 970 nm) wavelengths described above, and signals from the gyroscope and accelerometer. The sensor or module processor can output a plurality of physiological parameters based on the input signals from the sensors described above. The plurality of physiological parameters can include, for example, SpO2 (Sat), pulse rate (PR), perfusion index (PI), pleth variability index (PVI), respiration rate from the pleth (RRp), and a hydration index.

As shown in greater detail in FIG. 15B, the sensor or module processor can process the intensity signal in response to detected light of the first, second, and third wavelengths in the unnormalized form and a normalized form (in normalization modules “Norm” 1500, “Norm 1” 1502, and “Norm 2” 1504). As described above, the signal of the third wavelength can be used as the normalizing signal. The sensor or module processor can extract various information from the intensity signals in response to detected light of the first, second, and third wavelengths and signals from the accelerometer and the gyroscope, such as the PR (which can be output as the PR measurement), time domain (TD) saturation information, frequency domain (FD) saturation information, PI information, and PVI information, in a pulse rate determination module 1506.

FIG. 15C illustrates example processing of the raw signals from the accelerometer and the gyroscope to output the gyroscope and accelerometer signals. The sensor or module processor can combine each of the raw gyroscope and accelerometer signals (which can be raw signals from any axis of the gyroscope and/or accelerometer) with gyroscope/accelerometer time instants and pleth time instants signals in an interpolation module 1518 or interpolation 1 module 1520 respectively. The sensor or module processor can further process the outputs from the interpolation module 1518 or interpolation 1 module 1520 in a low pass filter and decimation module 1522 or low pass filter and decimation 1 module 1524 respectively to output a gyrol signal and an accelerometer 1 signal. The output gyre 1 and accelerometer 1 signals can be sent to the ASIC described above.

As shown in FIG. 15D, the sensor or module processor can extract motion information from the gyroscope and accelerometer input and the normalized signals of the first, second, and third wavelengths in an interference mitigation (IM) and motion analysis module 1526. As also shown in FIG. 15D, the sensor or module processor can obtain time domain pulse rate (TDPR) information, TD saturation information, PI information, and PVI information in a time domain pulse rate determination module 1528 from the intensity signals of the first, second, and third wavelengths. The sensor or module processor can obtain frequency domain pulse rate (FDPR) information and FD saturation information in a frequency domain pulse rate determination module 1530 based on normalized signals of the first, second, and third wavelengths. The sensor or module processor can determine and output a pulse rate in a pulse rate decision logic 1532 based on the TDPR information, FDPR information, interference mitigation (IM) PR information (output by the interference mitigation and motion analysis module 1526), and motion information.

FIG. 15E illustrates an example pulse rate determination decision logic. In this example, a decision logic stage 2 module 1534 can receive as input raw pulse rate calculations from individual pulse rate determination engines (for example, the time domain pulse rate determination module 1528, the frequency domain pulse rate determination module 1530 and the interference mitigation and motion analysis module 1526 as shown in FIG. 15D), pleth features including time domain and frequency domains from N channels (for example, N=4 or more) of pleth signals, and motion features obtained from a motion analysis module 1536. The motion analysis module 1536 can assess the amount of motion, define the type of motion, and calculate a motion rate (for example, per minute) if the motion is determined to be periodic, and/or the like based on motion information from a 6DOF (degree-of-freedom) inertia measurement unit (IMU). The IMU can include the accelerometer and the gyroscope on the physiological parameter measurement module.

With continued reference to FIG. 15B, the sensor or module processor can determine the oxygen saturation measurement based on the normalized signal of the third wavelength, the normalized signal of the second wavelength, the TD saturation information, the FD saturation information, the PR, and the motion information in an oxygen saturation determination module 1508. FIG. 15F illustrates an oxygen saturation determination module including a plurality of parallel signal processing engines, such as a Seed saturation module 1538, an SST saturation module 1540, a DST saturation module 1542, an interference mitigation (IM) saturation module 1544, and a signal/noise reference saturation module 1546, configured to feed individual raw oxygen saturation (SpO2) values to a decision logic 1548. The decision logic 1548 can further receive as input the motion information and output a final oxygen saturation measurement based on the motion information and the raw oxygen saturation values determined by the parallel engines.

FIG. 15E illustrates an example oxygen saturation determination decision logic. In this example, a saturation decision logic stage 2 module 1550 can receive as input raw oxygen saturation calculations from the parallel engines described above, pleth features, pulse rate, and motion features obtained from a motion analysis module 1552. The pleth features received by the module 1550 can include the features in the pulse rate decision logic shown in FIG. 15E. Additionally, the pleth features received by the module 1550 can include features related to saturation, for example, the DC ratio of the second and third wavelengths. The motion analysis module 1552 can receive the same features as the pulse rate decision logic shown in FIG. 15E.

With continued reference to FIG. 15B, the sensor or module processor can determine the PI measurement based on the normalized signal of the third wavelength and the PI information in a perfusion index determination module 1510. The sensor or module processor can determine the PVI measurement based on the PVI information in a pleth variability index determination module 1512. The sensor or module processor can determine the RRp measurement based on the intensity signals of the first and second wavelength in a respiration rate determination module 1514. The sensor or module processor can determine the hydration index in a hydration determination module 1516 based on the intensity signals (for example, from the “far detectors” disclosed herein) of the fourth wavelength, which is more sensitive to changes in water in the measurement site and another wavelength (for example, the third wavelength or about 905 nm) that is less sensitive to changes in water. The sensor or module processor can focus on the DC component of the signals for hydration status monitoring.

Various example physiological parameter measurement modules and wearable devices incorporating the same will be described below. Each of the example modules and devices can incorporate any of the features of the physiological parameter measurement module 100 and the device 10 described above, all of which are not repeated for brevity. Features of the example modules and devices disclosed herein can be incorporated into one another.

Examples of Physiological Parameter Measurement Modules with Double Emitter Groups

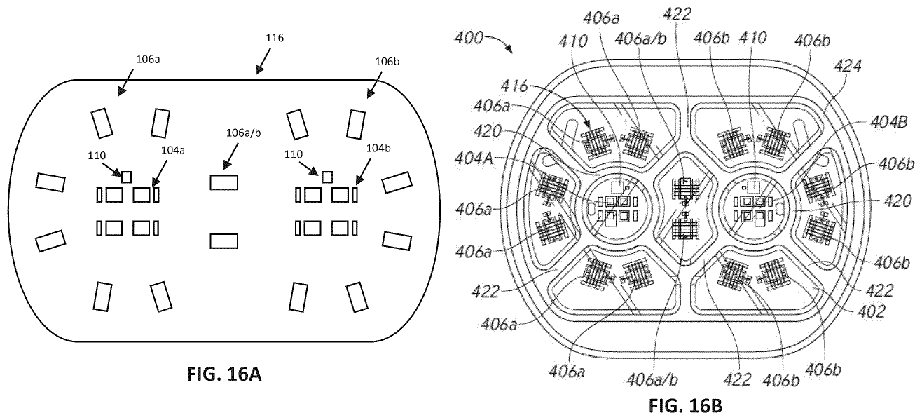

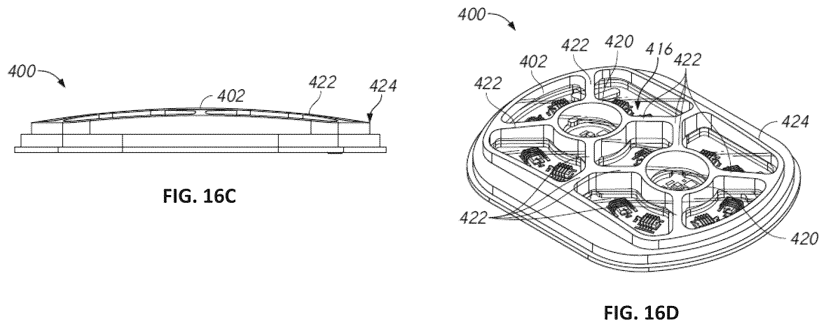

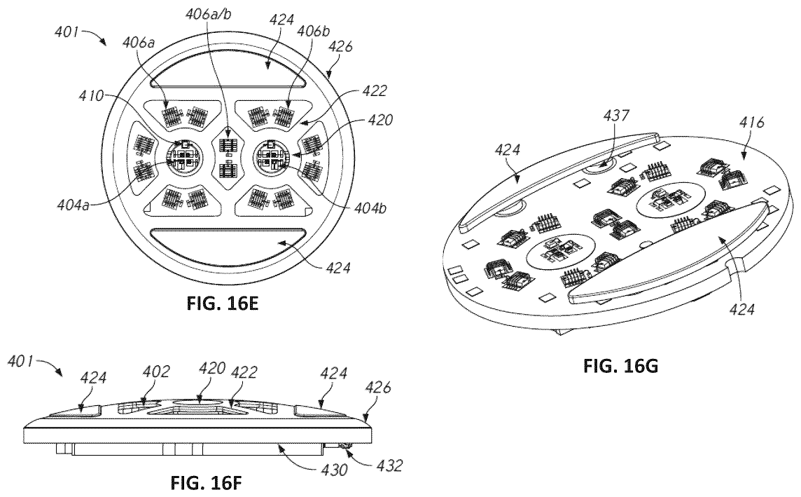

FIG. 16A illustrates schematically an example arrangement of an optical sensor, including emitters, detectors, and thermistors, on a sensor or module processor PCB 116. As shown in FIG. 16A, the PCB 116 can include a first group of emitters 104a and a second group of emitters 104b. Each group of emitters can include four emitters. The emitters in each group 104a, 104b can emit at least the first, second, third, and fourth wavelengths as described above. The first and second groups of emitters 104a, 104b can be located a distance from each other on a first side of a PCB 116. The PCB 116 can include a temperature sensor (such as a thermistor) 110 as described above located on the first side of the PCB 116. One temperature sensor 110 can be near the first group of emitters 104a. Another temperature sensor 110 can be near the second group of emitters 104b.

The PCB 116 can be elliptical in shape, although the shape of the PCB is not limiting. The two groups of the emitters 104a, 104b can be located on different parts of the first side of the PCB 116 divided along the minor diameter of the ellipse. Each of the two groups of the emitters 104a, 104b can be surrounded by a first light barrier and form an emitter chamber.

The first and second groups of emitters 104a, 104b can be surrounded by two rings of detectors 106a, 106b that are separated from the first and second groups of emitters 104a, 104b respectively by a distance. The two rings of detectors 106a, 106b can share a plurality of (for example, two or more) detectors 106a/b common to both rings. The detectors 106a/b common to both rings can be located along the minor axis of the ellipse. In the illustrated example, the PCB 116 can include fourteen detectors coupled to the PCB 116, but the total number of detectors can vary.

The detectors 106b can be the far detectors for the first group of emitters 104a and the detectors 106a, 106a/b can be the near detectors for the first group of emitters 104a. The detectors 106a can be the far detectors for the second group of emitters 104b and the detectors 106b, 106a/b can be the near detectors for the second group of emitters 104b. Accordingly, each detector 106a, 106b, 106a/b can receive two signals for each wavelength emitted by the first and second groups of emitters 104a, 104b respectively. As described above, signals outputted by the far and near detectors can provide different information due to the different light paths, which can travel through different areas of the tissue. In addition, the far detectors for each group of emitters 104a, 104b can detect the light emitted by the respective group of emitters 104a, 104b, for example, light of the fourth wavelength and another wavelength, and attenuated by tissue to provide an indication of the wearer's hydration status as described herein.

The detectors 106a, 106b, 106a/b can be separated or partitioned into seven detector regions. Each detector region can include two detectors, or any other number of detectors. Each detector region can form a detector chamber surrounded by light barriers. As described above, the sensor or module processor can process signals from a particular emitter and received at the detectors within the same detector region as one signal source. Accordingly, for each wavelength, the sensor or module processor can receive data from a total of fourteen signal sources, two from each detector region acting as the far and near detectors for the different groups of emitters respectively.

FIGS. 16B-16D illustrate an example physiological parameter measurement module 400 of a wearable device. The module 400 can incorporate any of the features of the module examples described herein.

As shown in FIG. 16B, the physiological parameter measurement module 400 can include a first group of emitters 404a and a second group of emitters 404b incorporating the arrangement shown in FIG. 16A. Each group of emitters can include four emitters (or optionally a different number of emitters, such as six or eight emitters). The emitters in each group 404a, 404b can emit at least the first, second, third, and fourth wavelengths as described above. Each of the two groups of the emitters 404a, 404b can be surrounded by a first light barrier 420 and form an emitter chamber.

The first and second groups of emitters 404a, 404b in the module 400 can be surrounded by two rings of detectors 406a, 406b that are separated from the first and second groups of emitters 404a, 404b by the first light barrier 420. The two rings of detectors 406a, 406b can share a plurality of (for example, two or more) detectors 406a/b common to both rings. The detectors 406a, 406b, 406a/b can have the same arrangement as the detectors shown in FIG. 16A. In the illustrated example, the module 400 can include fourteen detectors, but the module 400 can also include a different total number of detectors.

As shown in FIGS. 16B and 16D, the detectors 406a, 406b, 406a/b can be separated or partitioned into seven detector chambers by a portion of the first light barrier 420 and second light barriers 422. Each detector region can include two detectors, or any other number of detectors. Along an outer perimeter of the module 400, the detectors 406a, 406b, 406a/b can be enclosed within a module side wall 424. A sensor or module processor of the module 400 can process signals from a particular emitter and received at the detectors within the same detector region as one signal source as described above. The arrangement of emitters 104a, 104b and detectors 106a, 106b, 106a/b and the light diffusing materials encapsulating the emitters 104a, 104b and/or detectors 106a, 106b, 106a/b can improve the sensing coverage on the wearer's wrist, which has fewer capillaries per volume than the fingertip as described above. The aggregate light detecting area of the 106a, 106b, 106a/b in FIG. 16B, that is, the aggregate surface area of all the detector chambers, can occupy about 50% or more of the tissue-facing surface of the physiological parameter measurement module. The aggregate light detecting area in FIG. 16B can be, for example, greater than about 100 mm², or greater than about 125 mm², or about 150 mm², or about 165 mm². The aggregate light emitting area in FIG. 16B, that is, the aggregate surface area of both emitters chambers, can be, for example, greater than about 25 mm², or about 30 mm², or about 35 mm². Any other physiological parameter measurement module examples disclosed herein can have the same or substantially similar aggregate light detecting area and/or light emitting area as the module 400 shown in FIG. 16B.

On the first side of the PCB 416, the module 400 can be enclosed by a curved light transmissive cover 402 with a convex protrusion. As shown in FIG. 16C, the cover 402 can have a continuous curvature. The first and second light barriers 420, 422 are configured to be in contact with the first side of the PCB 416 at one end. At the other end, the height of the first and second light barriers 420, 422, and of the side wall 424 can generally follow the curvature of the cover 402. The side wall 424 can be shorter than the second light barrier 422. The height of the second light barrier 422 can increase from the perimeter of the module 400 toward a center of the module 400 until the second light barrier 422 merges with the first light barrier 420, which is the highest among the light barriers. The light barriers 420, 422 can extend to the tissue-facing surface of the cover 402 so that when the module 400 is pressed into the skin of the wearer of a device incorporating the module 400, the tissue-facing surfaces of the first and second light barriers 420, 422, and of the side wall 424 can be configured to contact the skin of the wearer. The cover 402 can include individual lenses or covers such as shown in FIG. 7D or a combination of individual emitter chamber covering lenses or covers and a lens or cover covering a plurality of detector chambers, such as shown in FIG. 7C. The lenses or covers may be polycarbonate. The tissue-facing surface of the module 400 can include a continuous convex curvature.

The first and second light barriers 420, 422 and the side wall 424 can optionally form a single light barrier construct. The single light barrier construct can be formed by any suitable manufacturing techniques and any suitable materials, for example, plastic, colored, or opaque sapphire glass, or others. The single light barrier construct can include at one end a recess that is shaped and sized to receive the PCB 416, including the electronics on the PCB 416. The first side of the PCB 416 can include the emitters 404a, 404b, detectors 406a, 406b, 406a/b, temperature sensor 410, and any other sensors, for example, the gyroscope, the accelerometer, and/or the like. The second side of the PCB 416 can include the sensor or module processor and other circuit hardware.

As described above, the module 400 can include a plurality of chambers such that light cannot travel between the chambers because of the various light barriers extending from the PCB 416 to the tissue-facing surface of the cover 402 as described herein. The light diffusing materials described above can be added above (for example, via the fill holes described herein) and around the emitters 404a, 404b, and/or optionally above and around the detectors 406a, 406b, 406a/b, to improve distribution of emitted lighted and/or detected light after attenuation by the tissue. The light diffusing materials can include a flow of glass microsphere solution, which can be injected into the chambers after the module 400 has been assembled. After being injected into the respective chamber, the solution can be UV-cured. Air can escape via the vent openings disclosed herein as the diffusing material solution is injected into the respective chambers via the injection openings, making it easier for the glass microsphere solution to flow into the respective chamber. The cover 402 can also include glass microspheres. The light diffusing materials in the cover 402 and inside the emitter chambers and/or the first light barrier 420 can make the emitted light leave the emitter chambers enclosing the emitters 404a, 404b in a direction generally parallel to the height of the first light barrier 420. The light diffusing materials in the cover 402 and the detector chambers can increase the amount of reflected light being directed to and detected by the detectors 406a, 406b, 406a/b.

FIGS. 16E-16G illustrate an example physiological parameter measurement modules 401 of a wearable device. The module 401 can include the same optical sensor arrangements as shown in FIGS. 16A-16D and have any of the features of the module 400 in FIGS. 16B-16D with the differences noted in the description of FIGS. 16E-16G. The module 401 can have any of the features of the other physiological parameter measurement module examples described herein.

The module 401 can include a generally circular outer shape. The generally circular outer shape can be defined by an opaque frame 426 extending over of the PCB 416 from a first side of the PCB 416. The opaque frame 426 can have a height such that a top side of the opaque frame 426 can be generally level with (or receding or protruding slightly from) a second side of the PCB 416. As shown in FIG. 16G, the PCB 416 can be generally circular in shape. The opaque frame 426 can be generally concentric with the PCB 416. The opaque frame 426 and the PCB 416 are not transmissive to light. The opaque frame 426 in FIGS. 16E and 16F can include the first light barrier 420 and second light barriers 422 as an integral piece.

The module 401 can include one or more (for example, two or otherwise) ECG electrodes 424. In the illustrated examples of FIGS. 16E-16G, one of the ECG electrodes 424 can be a reference electrode and the other one of the ECG electrode 424 can be a negative or positive electrode. The opaque frame 426 can have indentations having the shape and size to accommodate the electrodes 424, similar to the indentations on the opaque frame 126 shown in FIG. 7D. As shown in FIG. 16F, a bottom surface of the electrodes 424 can have a curvature that is generally continuous with the curvature of the opaque frame 426 and the light-transmissive cover 402. As shown in FIG. 16G, a top side of the electrodes 424 can have one or more posts 437 extending past openings in the opaque frame 426 into corresponding openings on the PCB 416. The posts 437 of the electrodes 424 can establish an electrical connection with the corresponding openings of the PCB 416. A plurality of screws (or other types of fasteners) can extend into the corresponding openings of the PCB 416 from the front side of the PCB 416 to secure the electrodes 424 to the module 401 by threadedly mating with the posts. When a wearer puts the wearable device incorporating the module 401 onto the wearer's wrist, the electrodes 424 can make contact with the wearer's skin. The electrodes 424 can have the same polarity as the electrodes 124 disclosed herein. As disclosed herein, the wearable device incorporating the module 401 can include another ECG electrode 125 located on the housing of the wearable device configured to make contact with the wearer's skin.

On the second side of the PCB 416, which faces away from the cover 402, the PCB 416 can be covered by melt plastic or other suitable electronics protective material 430 (similar to the protective material 130 disclosed herein) except that a flex connector 432 can remain exposed. The flex connector 432 can be configured to connect the module 401 electrically to the wearable device incorporating the module 401.

FIGS. 17A-17C illustrate an example physiological parameter measurement modules 403 of a wearable device. The module 403 can include the same optical sensor arrangements as shown in FIGS. 16A-16G and have any of the features of the module 400 in FIGS. 16B-16D and any of the features of the module 401 in FIGS. 16E-16G with the differences noted in the description of FIGS. 17A-17C. The module 401 can have any of the features of the other physiological parameter measurement module examples described herein.

As shown in FIGS. 17A-17C, the opaque frame 426 can include an opening fitted with the light transmissive cover 402. The cover 402 extending over emitter chambers or detector chambers formed by the light barriers 420, 422, 423 and the PCB 415 can include a single lens or cover. The cover 402 can be elliptical in shape. The cover 402 can have a continuous convex curvature. As shown in FIG. 17C, the light barriers 420, 422, 423 may not extend to the tissue-facing surface of the cover 402 and can extend to below the cover 402 such that when a wearer puts on a wearable device incorporating the module 402, the wearer's tissue comes into contact with the cover 402 and the electrodes 424, but not with any of the light barriers 420, 422, 423.

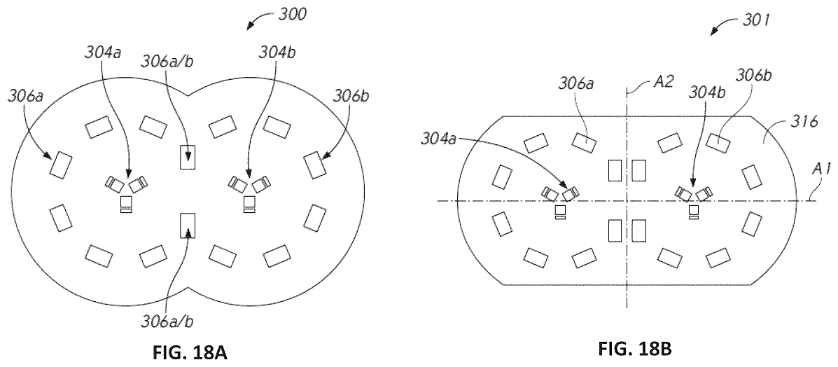

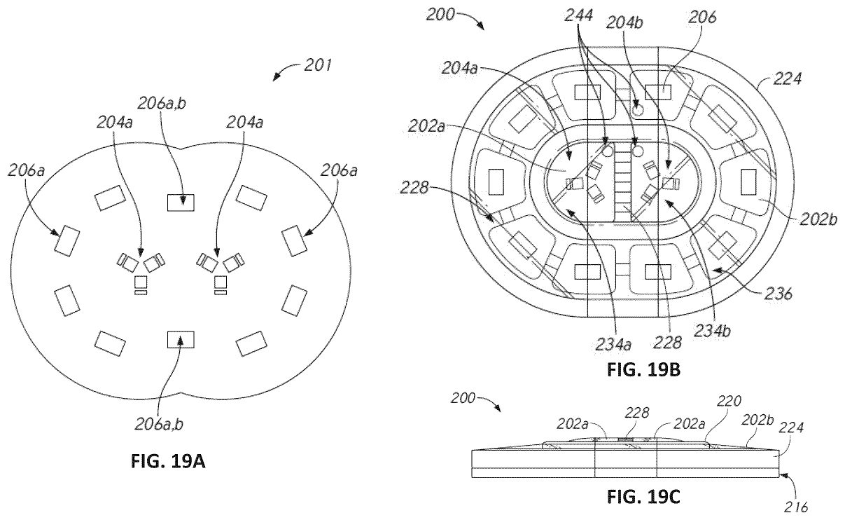

FIGS. 18A-19C illustrate other non-limiting examples of a physiological parameter measurement module with two emitter groups in two separate emitter chambers formed by a light barrier. In those configurations, the perimeter of the module can have a different shape. For example, FIG. 19A illustrates schematically a module 300 having an outer shape of two circles partially overlapped with each other. The circle in the module 300 can have a radius, for example, between about 6 mm and about 12 mm, or between about 8 mm and about 10 mm. The module 300 can have any of the features of the other modules disclosed herein. The module 300 can include the substantially the same arrangement of emitters 300a, 300b and detectors 306a, 306b, 306a/b as the module 400, 401, 403 described above except that each emitter group 304a, 304b includes three emitters. The module 300 can include a thermistor near each emitter group 304a, 304b. The module 300 can have a length of, for example, between about 22 mm and about 28 mm, or between about 24 mm and about 26 mm.

FIG. 18B illustrates a physiological parameter measurement module 301 including a variation of the arrangement of emitters and detectors of the module 300 in FIG. 18A, and can include any of the features of the module 300 except for the differences described herein. The module 301 differs from the module 300 by not sharing detectors located between the two groups of emitters 304a, 304b. The first group of emitters 304a can be surrounded by a first ring of detectors 306a on a first side of the minor axis A2 and the second group of emitters 304b can be surrounded by a second ring of detectors 306b that are on a second side of the minor axis A2.

FIG. 19A illustrates a physiological parameter measurement module 201 including a variation of the arrangement of emitters and detectors of the module 300 in FIG. 18A. The physiological parameter measurement module 201 can have any of the features of the modules 300, with the differences noted in the description of FIG. 19A. The module 201 can have any of the features of the other modules disclosed herein. In the module 201, the two overlapping circles of detectors 206a, 206b are closer to each other than in the module 300. The detectors 206a/b can be further away from each other than in the module 300 and may not be located between or separating the two emitter groups 204a, 204b. The module 201 can include two groups of emitters that are separated from each other by one light barrier. Each of the detectors in the module 201 can form its own detector chamber with one or more light barriers. The circle can have a radius, for example, between about 6 mm and about 12 mm, or between about 8 mm and about 10 mm. The module 300 can have a length of, for example, between about 18 mm and about 24 mm, or between about 20 mm and about 22 mm.

FIGS. 19B and 19C illustrate a variation of the module 201 in FIG. 19A with the differences noted in the description of FIGS. 19B and 19C. The module 200 in FIGS. 19B and 19C can have any of the features of the module examples described herein. In FIGS. 19B and 19C, a physiological parameter measurement module 200 can include two groups of emitters 204a, 204b surrounded by one ring of detectors 206. The module 200 can have a width, for example, between about 16 mm and about 22 mm, or between about 18 mm and about 20 mm. The module 200 can have a length, for example, between about 20 mm and about 28 mm, or between about 22 mm and about 25 mm.

Each group of the emitters 204a, 204b can include three of emitters. Each group of the emitters 204a, 204b can emit at least the first, second, and third wavelength described above. Optionally, each emitter group 204a, 204b can include a fourth emitter configured to emit the fourth wavelength that is more sensitive to water. The emitters can be located at or near a center portion of a PCB 216 of the module 200. The module 200 can include a temperature sensor located on the PCB 216 near each group of the emitters 204a, 204b.

The emitters can be covered by an inner lens or cover 202a. In the illustrated example, the inner lens or cover 202a can be generally elliptical. In other examples, the inner lens or cover may have any other shapes. The two groups of the emitters 204a, 204b can be located on two parts of the central portion of the PCB divided along the minor diameter of the ellipse. The two groups of the emitters 204a, 204b can be divided by an opaque divider barrier 228, which can reduce mixing of light emitted by the two groups of the emitters 204a, 204b. As shown in FIG. 19C, the divider barrier 228 can have a same or substantially the same height as the highest point of the inner lens or cover 202a when assembled in the module 200. The inner lens or cover 202a can include two components divided by the divider barrier 228.

The module 200 can include a plurality of detectors 206 (for example, about six, eight, ten, or more) that can be arranged on the PCB so that the detectors 206 are spaced apart around the emitters 204a, 204b. The emitters groups 204a, 204b and the detectors 206 can be separated by a first light barrier 220. The first light barrier 220 can extend along and surround the inner lens or cover 202a. The divider barrier 228 and the first light barrier 220 can form two emitter chambers 234a, 234b, each enclosing one of the two emitter groups 204a, 204b. The first light barrier 220 and the divider barrier 228 can also suppress light emitted by the emitters 204a, 204b at an angle so the light emitted by each group of emitters 204a, 204b can exit the inner lens or cover 202a in a direction generally parallel to the height of the first light barrier 220. The detectors 206 can be enclosed within a module side wall 224. The module side wall 224 can define a perimeter of the module 200. As shown in FIG. 19B, the perimeter of the module 200 can have a generally elliptical outer shape. The detectors 206 can be further separated from one another by a plurality of divider barriers 226, forming detector chambers 236, each containing one detector 206.

As shown in FIG. 19C, the first light barrier 220 can protrude slightly from, that is, proud of the edge of the inner lens or cover 202a and the other lenses or covers that will be described below. The detectors 206 can be covered by an outer lens or cover 202b. The outer lens or cover 202b can be generally concentric with the inner lens or cover 202a. In the illustrated examples, the outer lens or cover 202b can be an elliptical disc as shown in FIG. 19B. In other examples such as those disclosed herein, the outer lens or cover can have other shapes. As shown in FIG. 19C, the outer lens or cover 202b can have a smaller curvature than the inner lens or cover 202a such that the inner lens or cover 202a protrudes more than if the inner lens or cover had the same curvature as the outer lens or cover 202b.

As shown in FIG. 19C, the side wall 224 can be shorter than the first light barrier 220. The height of the side wall 224 can be configured such that the tissue-facing end of the side wall 224 is generally continuous with the curvature of outer lenses or covers 202b. The divider barriers 226 can have a height lower than the first light barrier 220. The height of the divider barriers 226 can be configured to accommodate the outer lens or cover 202b such that when assembled, the outer lens or cover 202b forms a substantially smooth surface with the module side wall 224. The tissue-facing ends of the first light barrier 220 and the side wall 224, and the tissue-facing surfaces of the inner lens or cover 202a and the outer lens or cover 202b can form the tissue-facing surface of the module 200. The slightly protruding first light barrier 220 and/or inner lens or cover 202a can be pressed into the wearer's skin at a higher pressure than the remainder of the lens or cover or light barriers.

The light diffusing materials described above can be included in one or more of the chambers 234a, 234b, 236 of the module 200 to improve distribution of emitted lighted and/or detected light. As shown in FIG. 19B, one or more of the lenses or covers 202a, 202b can include an injection opening 244 so that the light diffusing materials, which can include a flow of glass microsphere solution, can be injected into the respective chambers 234a, 234b, 236 after the module 200 has been assembled. After the injection, the solution can be UV-cured. The lenses or covers 202a, 202b can include one or more venting openings that are smaller than the injection openings 244. Air can optionally escape via separate vent openings as the diffusing material solution is injected into the respective chambers 234a, 234b, 236 via the injection openings 244. The inner lens or cover 202a and the outer lens or cover 202b can also include glass microspheres so as to act as light diffusers.

FIGS. 27A-27J illustrate other non-limiting examples of a physiological parameter measurement module 2700 of a wearable device. The module 2700 can incorporate any of the features of the module examples described herein. FIG. 27F illustrates an example arrangement of an optical sensor, including emitters, detectors, and thermistors, on a sensor or module processor substrate 2716. The substrate 2716 can include a printed circuit board (PCB). As shown in FIG. 27F, the substrate 2716 can include a first group of emitters 2704a and a second group of emitters 2704b. Each of the first and second groups of emitters 2704a, 2704b can include four emitters (or optionally a different number of emitters, such as three, six or eight emitters or any other number of emitters as required or desired). Each of the emitters of the first and second groups of emitters 2704a, 2704b may comprise an LED.

The emitters in the first and second group of emitters 2704a, 2704b can include operational and/or structural features discussed above, for example with reference to FIGS. 13A-13B. For example, the first and second group of emitters 2704a, 2704b can be configured to emit a plurality of (for example, three, four, or more) wavelengths. In some aspects, each of the emitters (e.g., within a group) can be configured to emit light of a different wavelength than the other emitters (e.g., of that group). Alternatively, one or more of the emitters can emit light of more than one wavelength. The emitters in the first and second group of emitters 2704a, 2704b can emit at least the first, second, third, and fourth wavelengths as described above with reference to FIG. 13B.

Each of the emitters in the first group of emitters 2704a may be located within close proximity to each of the other emitters in the first group of emitters 2704a. For example, each of the emitters of the first group of emitters 2704a may be located on the PCB between 0.2 mm and 2 mm from each of the other emitters in the first group of emitters 2704a. For example, each of the emitters of the first group of emitters 2704a may be located on the PCB about 0.5 mm from each of the other emitters of the first group of emitters 2704a. In some aspects, each of the emitters in the first group of emitters 2704a are positioned such that no more than a certain distance is between each of the emitters in the first group of emitters 2704a, such as 0.5 mm, 1 mm, 1.5 mm and/or 2 mm, or any other distance as required or desired. Each of the emitters in the second group of emitters 2704b may be located within close proximity to each of the other emitters in the second group of emitters 2704b, for example as described above with reference to the first group of emitters 2704a.

Each of the two groups of the emitters 2704a, 2704b can be surrounded by a first light barrier and form emitter chambers for the group of emitters 2704a and group of emitters 2704b, respectively. The first and second groups of emitters 2704a, 2704b can be located a distance from each other on a first side of a substrate 2716. The substrate 2716 can include one or more temperature sensor(s) (such as a thermistor) 2710 as described above located on the first side of the substrate 2716. One temperature sensor 2710 can be near the first group of emitters 2704a within the respective emitter chamber. Another temperature sensor 2710 can be near the second group of emitters 2704b within the respective emitter chamber.

The substrate 2716 can be circular in shape, although the shape of the PCB is not limiting. The two groups of the emitters 2704a, 2704b can be located on different parts of the first side of the substrate 2716 divided along a center line of the circle. Each of the two groups of the emitters 2704a, 2704b can be surrounded by a first light barrier and form an emitter chamber.

The first and second groups of emitters 2704a, 2704b can be surrounded by detectors 2706. As described in greater detail with reference to FIG. 27A, the detectors 2706 can be positioned on the substrate 2716 in a substantially circular or annular arrangement. The detectors 2706 may surround and/or enclose the first and second groups of 2704a, 2704b. Each of the detectors may be a similar or same distance from a geometric center of the substrate 2716 and/or the sensor or module 2700. In some aspects, the detectors 2706 may be rectangular including longer sides and shorter sides. The detectors 2706 may be positioned on the substrate 2716 such that a long side of each detector is orthogonal to a radius of the substrate 2716. In the illustrated example, the substrate 2716 includes six detectors coupled to the substrate 2716, but the total number of detectors can vary.

The detectors 2706b can be the far detectors for the first group of emitters 2704a. The detectors 2706a can be the near detectors for the first group of emitters 2704a. The detectors 2706a can be the far detectors for the second group of emitters 2704b. The detectors 2706b can be the near detectors for the second group of emitters 2704b. The detectors 2706c can be the intermediate detectors for the first and second groups of emitters 2704a, 2704b. Accordingly, each detector 2706a, 2706b, 2706c can receive two signals for each wavelength emitted by the first and second groups of emitters 2704a, 2704b respectively. As described above, signals outputted by the far, near and intermediate detectors can provide different information due to the different light paths, which can travel through different areas of the tissue.

In some aspects, the sensor or module processor may evaluate the various signals outputted by the detectors for example by comparing the signal quality of the detectors. The sensor or module processor may select less than all of the detector signals for processing for each of the far, near and intermediate detectors. For example, the sensor or module processor may rely on signals from one or two detectors from the four possible far detectors, and one or two detectors from the four possible near detectors, and one or two detectors from the four possible intermediate detectors.

In addition, the far detectors for each group of emitters 2704a, 2704b can detect the light emitted by the respective group of emitters 2704a, 2704b, for example, light of the fourth wavelength and another wavelength, and attenuated by tissue to provide an indication of the wearer's hydration status as described herein.

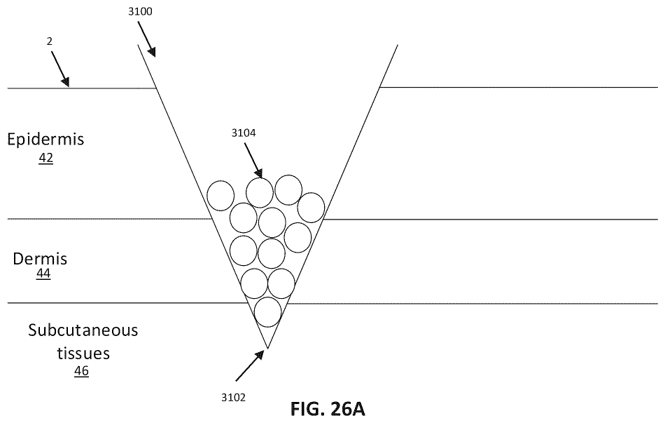

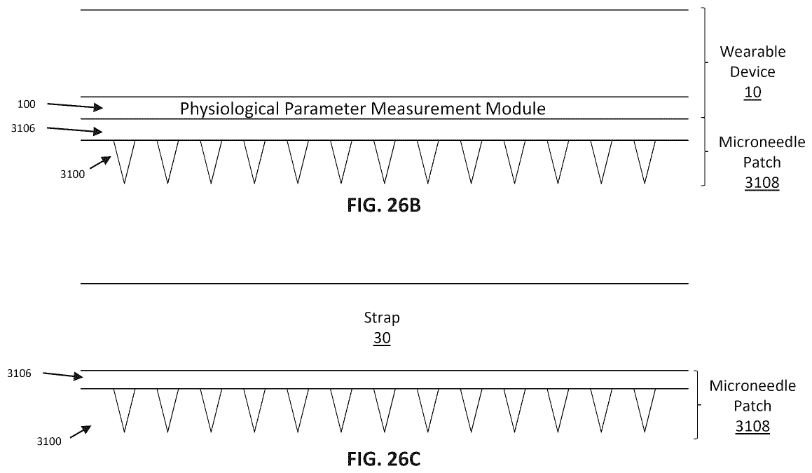

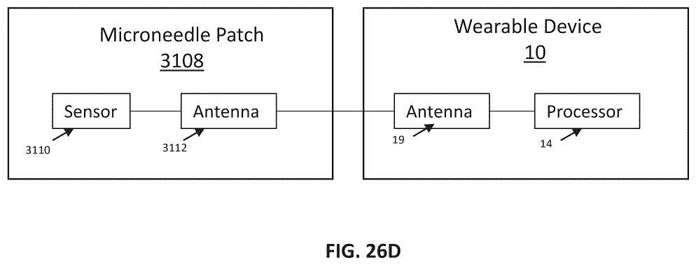

The detectors 2706a, 2706b, 2706c can be separated or partitioned into six detector regions. Each detector region can include one detector, or any other number of detectors. Each detector region can form a detector chamber surrounded by light barriers (as shown in FIG. 26E). As described above, the sensor or module processor can process signals from a particular emitter and received at the detectors within the same detector region as one signal source.

The module 2700 can include individual lenses or covers or a combination of individual emitter chamber covering lenses or covers and a lens or cover covering a plurality of detector chambers. The lenses or covers may be polycarbonate. The tissue-facing surface of the module 2700 can include a continuous convex curvature.

FIG. 27I illustrates another example arrangement of an optical sensor, including emitters, detectors, and thermistors, on a sensor or module processor substrate 2716′. The substrate 2716′ of FIG. 27I can include structural and/or operational features similar to those discussed above with reference to FIG. 27F. As shown in FIG. 27I, each of the first and second groups of emitters 2704a′, 2704b′ can include five emitters (or optionally a different number of emitters as required or desired). Each of the emitters of the first and second groups of emitters 2704a′, 2704b′ may comprise an LED and can be configured to emit light at various wavelengths such as any of the wavelengths discussed herein, for example, a first wavelength of about 525 nm to about 650 nm (such as about 525 nm or about 580 nm or about 645 nm), a second wavelength from about 620 nm to about 660 nm (such as about 625 nm), a third wavelength from about 650 nm to about 670 nm (such as about 660 nm), a fourth wavelength from about 900 nm to about 910 nm, and a fifth wavelength at about 970 nm.

As shown in FIG. 27I, the substrate 2716′ can include spring contacts 2755′ for facilitating physical and/or electrical connection between the substrate 2716′ and electrodes (e.g., electrodes 2724 shown in FIG. 27B, for example).

As shown in FIGS. 27G-27H, on the second side of the substrate 2716, which faces away from the cover, the substrate 2716 can be covered by melt plastic or other suitable electronics protective material 2730 (similar to the protective material 130 disclosed herein) except that a flex connector 2732 can remain exposed. The flex connector 2732 can be configured to connect the module 2700 electrically to the wearable device incorporating the module 2700.

FIG. 27J illustrates a perspective cross-sectional view of an example physiological parameter measurement sensor or module 2700. As shown, the sensor or module 2700 can include a first circuit board (e.g., substrate 2716) and a second circuit board (e.g., instrument PCB 2768. The sensor or module 2700 can include an induction coil 2765. The induction coil 2765 can be electrically and/or physically connected to the substrate 2716, for example via solder and/or a flex connector connection 2762. The induction coil can provide a voltage to the substrate 2716, for example via the solder and/or flex connector connection 2762. The substrate 2716 can include charging circuitry which can be configured to receive and/or control a voltage received from the induction coil 2765. The substrate 2716 can be configured to transfer voltage received from the induction coil 2765 to the instrument PCB 2768, for example via a flex connector 2761. The instrument PCB 2768 can be configured to transfer voltage received from the substrate 2716 to a battery or to other components of the sensor or module 2700 as required or desired.

Examples of Physiological Parameter Measurement Modules with Inner and Outer Detector Groups and Examples of Wearable Devices Incorporating the Same

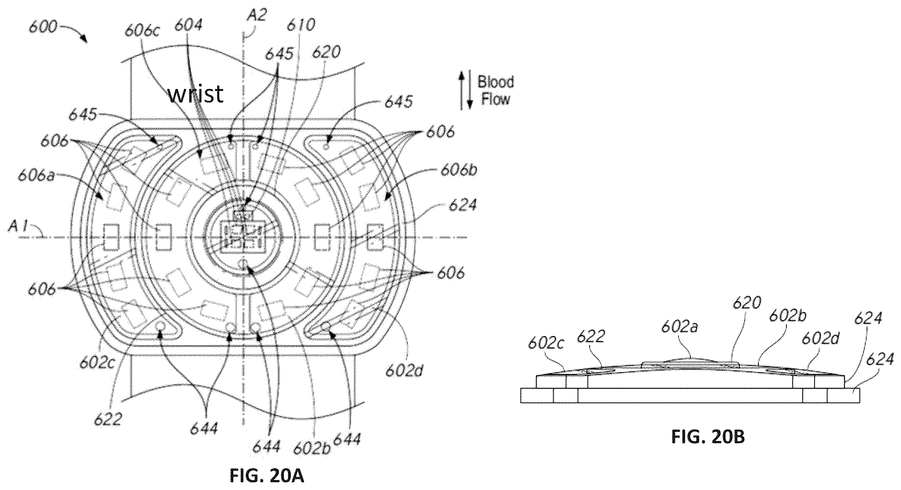

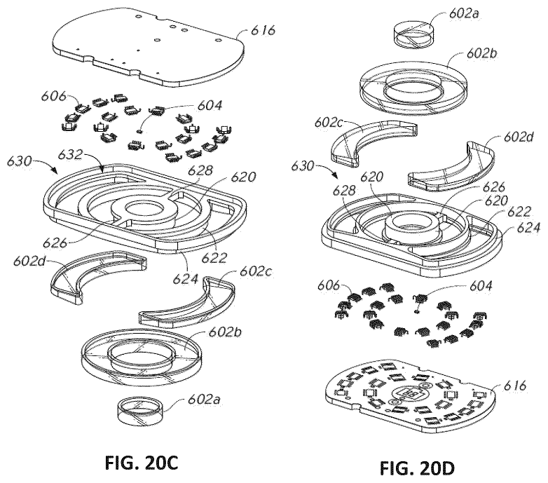

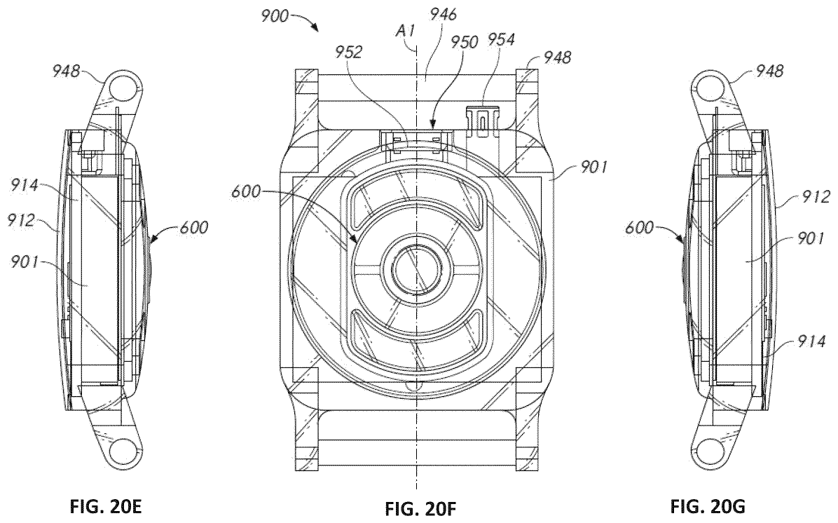

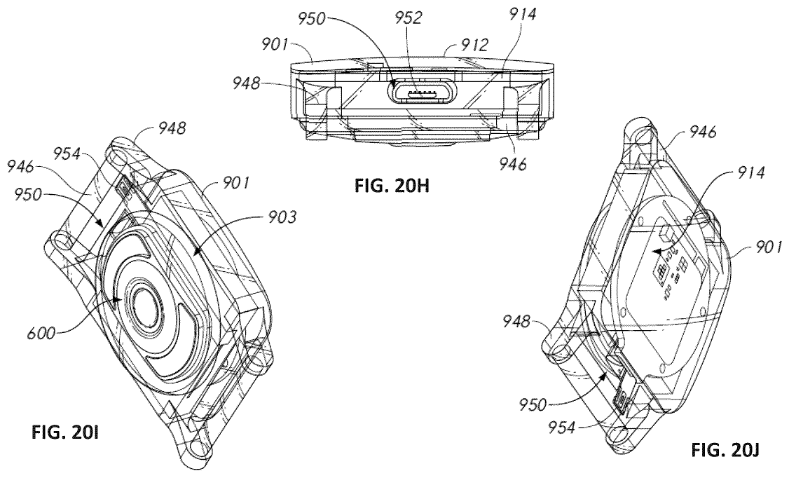

FIGS. 20A-20D illustrate an example physiological parameter measurement module 600 of a wearable device. The module 600 can have any of the features of the module examples described herein, with the differences noted in the description of FIGS. 20A-20D. The physiological parameter measurement module 600 can include a single emitter group having a plurality of emitters 604, such as four emitters as shown in FIG. 20A, six emitters, or eight emitters. The emitters 604 of the module 600 can emit at least the first, second, third, and fourth wavelengths as described above. The emitters 604 can be located at or near a center portion of a PCB 616 of the module 600. The module 600 can include a temperature sensor 610 located on the PCB 616 near the emitters 604.

The module 600 can include a plurality of detectors 606 that can be arranged on the PCB 616 as an inner group of detectors 606 and an outer group of detectors 606. The inner group 606c of detectors 606, which can include, for example, about ten (or a different number of) detectors 606, can surround the emitters 604 and be spaced apart from one another.

The outer group of detectors 606 can be located further away from the emitters 604 than the inner group of detectors 606. The outer group of detectors 606 can be separated into a first outer group 606a and a second outer group 606b of detectors 606. As shown in FIG. 20A, the module 600 can have a first axis A1 and a second axis A2. The outer groups 606a, 606b of detectors 606 can be located further away from the emitters 204 than the inner group of detectors 606 generally along the first axis A1. The two outer groups 606a, 606b of detectors 606 are on opposite sides of the inner group of detectors along the first axis A1. The first and second outer groups 606a, 606b of detectors 606 can be generally symmetrical about the first axis A2 and the second axis A2. Each of the first or second outer groups 606a, 606b of detectors 606 can include about five (or a different number) of detectors 606 that are spaced apart from one another generally along the second axis A2. The outer groups 606a, 606b of detectors 606 can be arranged to be generally concentric with the inner group 606c of detectors 606.

The module 600 can be longer in the first axis A1 than in the second axis A2. The module 600 can have a dimension of about 25.4 mm (1 inch) along the first axis A1. The module can have a dimension of about 19.1 mm (0.75 inch) along the second axis A2. As shown in FIG. 20A, when a watch incorporating the module 600 is worn on the wrist of a wearer, the first axis A1 can be generally parallel to the width of the wrist and generally perpendicular to the direction of blood flow along the wrist (that is, along a direction between the hand and the forearm) and the second axis A2 can be generally perpendicular to the width of the wrist and generally parallel to the direction of blood flow along the wrist. The distribution of the detectors 606 along the first axis A1 can improve detection of the light attenuated by the pulsing arterial blood in the capillaries as the detectors 606 are arranged to cover a greater cross-section of the blood flow through the wrist. Similarly, in the other example modules described herein, such as the sensor or module 100, 400, 401, 403, 300, 301, 200, 201, the physiological parameter measurement module is incorporated in the wearable device such that the longer side of the module is generally perpendicular to the direction of the blood flow along the wrist (see, for example, FIG. 1B) when the wearable device is worn on the wrist.

As shown in FIG. 20A, the emitters 604 can be covered by an inner lens or cover 602a. In the illustrated example, the inner lens or cover 602a can be generally circular. In other examples such as disclosed herein, the inner lens or cover may not be generally circular, but can have other shapes, for example, elliptical, rectangular, square, diamond, or otherwise. The inner group 606c of detectors 606 can be covered by a first outer lens or cover 602b. The first outer lens or cover 602b can be generally concentric with the inner lens or cover 602a. In the illustrated example, the first outer lens or cover 602b can be disc shaped. The first and second outer groups 606a, 606b of detectors 606 can be covered by a second outer lens or cover 606c and a third outer lens or cover 606d respectively. The second and third outer lenses or covers 606c, 606d can be symmetrical about the second axis A2. As shown in FIG. 20B, the first, second, and third outer lenses or covers 602b, 602c, 602d can have substantially the same curvature. The inner lens or cover 602a can be more curved than the outer lenses or covers 602b, 602c, 602d such that the inner lens or cover 602a protrudes more than if the inner lens or cover 602a had same curvature as the outer lenses or covers 602b, 602c, 602d.

The inner group 606c of detectors 606 and the emitters 604 can be separate by a first light barrier 620. The first light barrier 620 can extend along and surround the inner lens or cover 602a, forming an emitter chamber. The first and second outer groups 606a, 606b of detectors 606 can be separated from the inner group 606c of detectors 606 by a second light barrier 622. The second light barrier 622 can be shorter than the first light barrier 620. The first and second outer groups 606a, 606b of detectors 606 can be enclosed within a module side wall 624 enclosing a perimeter of the module 600. The perimeter of the module 600 can be elliptical or any other shape. The side wall 624 can be shorter than the second light barrier 622. The height of the first and second light barriers 620, 622, and of the side wall 624 can generally follow or be substantially continuous with the curvature of the first, second, and third outer lenses or covers 602b, 602c, 602d. The first and second light barriers 620, 622, and of the side wall 624 can have a height so as to be configured to contact the skin of the wearer. Accordingly, the tissue-facing surface of the module 600 can be defined by the tissue-facing side of the first and second light barriers 620, 622, and of the side wall 624 and tissue-facing surfaces of the inner lens or cover 602a and the first, second, and third outer lenses or covers 602b, 602c, 602d.

In the illustrated example, the inner group 606c of detectors 606 can be separated by a third light barrier 626 and a fourth light barrier 628 (see FIGS. 20C and 20D). The third and fourth light barriers 626, 628 can have a height lower than the first light barrier 620 or the second light barrier 622. The height of the third and fourth light barriers 626, 628 can be configured to accommodate the first outer lens or cover 602b such that when assembled, the first outer lens or cover 602b forms a substantially smooth surface with the second and third outer lenses or covers 602c, 602d. The first outer lens or cover 602b can sit on top of the third and fourth light barriers 626, 628.

The first light barrier 620 can protrude slightly from, that is, sit proud of the edge of the inner lens or cover 602a and the outer lenses or covers 602b, 602c, 602d. The slightly protruding first light barrier 620 and/or inner lens or cover 602a can be pressed into the wearer's skin at a higher pressure than the remainder of the lenses or covers or light barriers. The first light barrier 620 can also reduce mixing of the emitted and reflected light and/or suppress light emitted by the emitters 604 at an angle so that the emitted light exits the inner lens or cover 602a generally in a direction parallel to the height of the first light barrier 620.

As shown in FIGS. 20C and 20D, the first, second, third, and fourth light barriers 620, 622, 626, 628 and the side wall 624 can optionally form a single light barrier construct 630. The single light barrier construct 630 can be formed by any suitable manufacturing techniques. The single light barrier construct 630 can include at one end a recess 632 (see FIG. 20C) that is configured to receive the PCB 616 (and the emitters 604, detectors 606, temperature sensor 610, and any other sensors, for example, the gyroscope, the accelerometer, and/or the like, and the sensor or module processor, which are located on the PCB 616). The single light barrier construct 630 can receive the lenses, including the inner lens or cover 602a, the first, second, and third outer lenses or covers 602b, 602c, 602d at another end that is opposite to the end including the recess 632.

The module housing can include a plurality of chambers such that light cannot travel between the chambers because of the various light barriers described herein. As described above, the first chamber can be enclosed by the inner lens or cover 602a, the first light battier 620, and a portion of the PCB 616. The first chamber 634 enclose the emitters 604. A second chamber and a third chamber can be enclosed by the first outer lens or cover 602b, the first light barrier 620, the second light barrier 622, the third light barrier 626, the fourth light barrier 628, and a portion of the PCB 616. The second and third chambers can enclose the inner group 606c of detectors 606, with half of the inner group 606c of detectors enclosed by each of the second and third chambers. A fourth chamber can be closed by the second outer lens or cover 602c, the second light barrier 622, the side wall 624, and part of the PCB 616. A fifth chamber can be enclosed by the third outer lens or cover 602d, the second light barrier 622, the side wall 624, and part of the PCB 616. The fourth and fifth chambers can enclose the first and second outer groups 606a, 606b of detectors 606 respectively.

Light from the emitters 604 can travel a shorter path to the inner group 606c of detectors 606 and a longer path to the first and second outer groups 606a, 606b of detectors 606. The inner group 606c of detectors 606 and the first and second outer groups 606a, 606b of detectors 606 can be run independently and/or simultaneously. Signals outputted by the inner and outer groups 606a, 606b of detectors 606 can provide different information due to the different light paths, which can travel through different areas of the tissue. The longer path penetrates deeper into the tissue and through a greater volume of the tissue to reach one of the outer groups 606a, 606b of detectors 606 than the short path, which penetrates less deep into the tissue and travels through a smaller volume of tissue to reach one of the inner group 606c of detectors 606. The different information can be separated and/or combined to calculate a plurality of physiological parameters of the wearer of the module 600, for example, an indication of the wearer's hydration status, which will be described in greater detail below.

The light diffusing materials described above can be included in one or more chambers of the module 600 to improve distribution of emitted lighted and/or detected light after attenuation by the tissue. As shown in FIG. 20A, one or more of the lenses or covers 602a, 602b, 602c, 602d can include an injection opening 644 so that the light diffusing materials, which can include a flow of glass microsphere solution, can be injected into the respective chambers after the module 600 has been assembled. After being injected into the respective chamber, the solution can be UV-cured. The lenses or covers 602a, 602b, 602c, 602d can include one or more venting openings 645 that are smaller than the injection openings 644. Each of the lenses or covers can include at least one venting opening 645. Air can escape via the vent openings 645 as the diffusing material solution is injected into the respective chambers via the injection openings 644, making it easier for the glass microsphere solution to flow into the respective chamber. The inner lens or cover 602a and/or the outer lenses or covers 602b, 602c, 602d can also include glass microspheres. The light diffusing materials in the inner lens or cover 602a and the UV-cured material in the first chamber 634 and/or the first light barrier 620 can make the emitted light leave the first chamber 634 in a direction generally parallel to the height of the first light barrier 620. The light diffusing materials in the outer lenses or covers 602b, 602c, 602d and the UV-cured material in the other chambers 636, 638, 640, 642 can increase the amount of reflected light being directed to the detectors 606.

The module 600 shown in FIGS. 20A-20D can be incorporated in a wearable device disclosed herein, such as a watch 900 shown in FIGS. 20E-20J. The watch processor 914 and power source can be enclosed within the watching housing 901. The watch housing 901 can include a connection port opening 950 configured to allow access to a connection port 952 that is in electrical communication with the watch processor 914 and/or the power source. The connection port opening 950 can be located at one end of the watch housing 901 transverse to the first axis A1 of the module 600. The connection port 952 can allow for charging of the power source and/or data transfer to and from the watch processor 914. Optionally, as shown in FIGS. 20F and 20I, the watch 900 can include a cable connector 945 extending outward from the watch housing 901. The cable connector 945 can be located adjacent to or near the connection port opening 950.

The watch 900 can include a display screen 912 positioned at a first side of the watch housing 901. The watch housing 901 has a second side that is opposite the first side. The second side of the watch housing 901 can include an opening sized to retain the physiological parameter measurement module 600 while still allowing the tissue-facing surface of the module 600 to be exposed. The second side of the watch housing 901 can be removably attached to the first side of the watch housing 901 without using external fasteners or alternatively via one or more fasteners. An electrical connection can be established between the physiological parameter measurement module PCB and the watch circuit, for example, using a flex connector as disclosed herein.

The watch housing 901 can include strap coupling extensions 948 on opposing sides of the watch 900 along the length of the housing 901 (that is, along the first axis A1 of the module 600). The extensions 948 can include a bar 946 for coupling to any suitable watch straps.

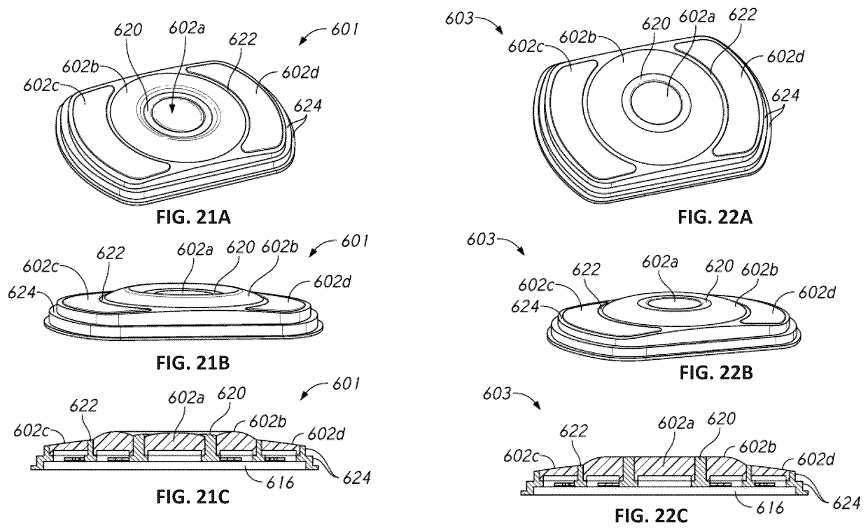

FIGS. 21A-21C and 22A-22C illustrate alternative lens or cover curvatures of the physiological parameter measurement module 600 of FIGS. 20A-20D and can incorporate any of the features of the module 600 of FIGS. 20A-20D except the differences described below. As shown in FIGS. 21A-21C, the first outer lens or cover 602b of the module 601 can be more convex (that is, protrude more) than the inner lens or cover 602a the second and third outer lenses or covers 602c, 602d. The curvatures of the tissue-facing side of the second light barrier 622 and of the side wall 624 can be substantially continuous with the curvature of the second and third outer lenses or covers 602c, 602d. The second light barrier 622 can be shorter than the first light barrier 620. The first light barrier 620 can be higher than an outer edge of the inner lens or cover 602a, which can facilitate separation of light emitted by the emitters 604 and light being detected by the detectors 606 before the light is attenuated by the wearer's body tissue. In the FIGS. 22A-22C, the module 603 can be different from the module 601 in FIGS. 21A-21C in that the inner lens or cover 602a can have the same height as the first light barrier 620 and the first outer lens or cover 602b. The inner lens or cover 602a can have a generally flat surface or a slight curvature that can be substantially continuous from the curvature of the first outer lens or cover 602b. The module 601, 603 in FIGS. 21A-22C can facilitate pressing the first outer lens or cover 602b or the first outer lens or cover 602b and the inner lens or cover 602a into the skin of the wearer more than the remainder of the tissue-facing surface of the module 600.

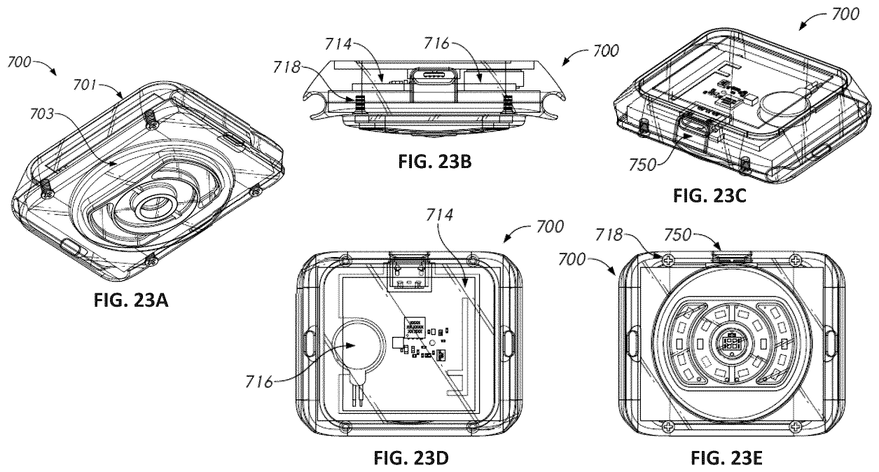

FIGS. 23A-23E illustrate a watch 700 that can incorporate the physiological parameter measurement module 600. The watch 700 can have any of the features of the watch 900 with the differences noted in the description of FIGS. 23A-23E. As shown in FIGS. 23A-23E, the watch housing 701 of the watch 700 can include a flap 750 on a side of the housing 701 along a length of the watch housing 701, which is along the first axis A1 of the physiological parameter measurement module (see FIG. 23E). The flap 750 can be opened to give access to a connection port (such as the connection port in the watch 900) in electrical communication with the watch processor 714 and/or the power source 716. The connection port can allow for charging of the power source 716 and/or data transfer to and from the watch processor 714. The flap 750 can be closed when the connection port 752 is not in use.

The watch 700 can include a display screen positioned at a first side of the watch housing 701. The watch housing 701 has a second side that is opposite the first side. The second side of the watch housing 701 can include an opening sized to retain the physiological parameter measurement module 600 while still allowing the tissue-facing surface of the module 600 to be exposed. The second side of the watch housing 701 can be removably attached to the first side of the watch housing 701 via one or more screws 718 or other fasteners. When fully assembled, the watch 700 can have a thickness or height, for example, between 10 mm to about 15 mm, or between 12 mm to about 14 mm.

The watch housing 701 can include suitable strap connections configured to couple to watch strap(s). The strap connections in the watch housing 701 can be different from the strap connections shown in the watch 900. In an example, a plurality of strap openings can be at opposite ends of the watch and the watch housing can additionally and/or alternatively include strap slots on the same opposite ends of the watch as the strap openings. In this example, the strap slots can be configured to slidably receive ends of watch straps that include a shape corresponding to the shape of the strap slots. The strap openings can be configured to receive spring-biased buttons near the ends of the watch straps to releasably retain the straps after the ends of the watch straps are received into the strap slots. Alternatively, the watch may not include strap openings. The strap(s) coupled to the watch examples disclosed herein can be configured to allow adjusting of tightness around the wearer's wrist, for example, using a buckle connector, a Velcro connector, and/or the like.

Hydration Monitoring by Wearable Devices Incorporating Examples Physiological Parameter Measurement Modules with “Near” and “Far” Detectors or Detector Groups

The physiological parameter measurement module examples disclosed herein can monitor a hydration status of the wearer. This is because water in the body tissue can allow a greater portion of the light of the third (or first or second) wavelength disclosed herein to go through (that is, acting as a light pipe), but can bulk absorb the light of the fourth wavelength disclosed herein. The physiological parameter measurement processor can compare intensity signals of the fourth wavelength and another wavelength that is less sensitive to changes in water from the same detector(s). When the wearer's hydration status is in a normal range such that the wearer is not considered dehydrated in a medical sense, the signals of the fourth wavelength and the other wavelength can show opposite trends, that is, one is increasing when the other one is decreasing. When the wearer becomes dehydrated in a medical sense, the opposite trends can become less distinct, for example, by falling below a threshold.

Hydration monitoring can be performed when the physiological parameter measurement module, such as the sensor or module 100, is configured such that at least some of the detectors 106 are located further away (far detector) from one of the emitters 104 (or emitter groups than the other detectors 106 (near detector), such as illustrated in FIG. 10. In configurations where there are two emitter groups, each detector 106 or detector region (which can include more than one detector 106 placed enclosed in the same detector chamber) can act as a near (or shallow) detector or detector region for the group of emitters that are closer to that detector 106 or detector region and as a far (or deeper) detector or detector region for the group of emitters that are further away from that detector 106 or detector region.

The physiological parameter measurement module 400, 401, 403 illustrates an example configuration for hydration monitoring of the wearer. The detectors 406a can be the far detectors for the second group of emitters 404b and the detectors 406b, 406a/b can be the near detectors for the second group of emitters 404b. The detectors 406b can be the far detectors for the first group of emitters 404a and the detectors 406a, 406a/b can be the near detectors for the first group of emitters 404a. The physiological parameter measurement modules 300, 301 illustrate similar detector arrangements in configurations (except that in the module 301, there are no shared detectors between the two groups of emitters 304a, 304b) where the modules 300, 301 include a fourth emitter in at least one of the emitter groups configured to emit light of the four wavelength.

The physiological parameter measurement modules 200, 201 illustrate additional example detectors configurations that can include “near” detectors for one emitter group and “far” detectors for another emitter group, in configurations where the modules 200, 201 include a fourth emitter configured to emit light of the fourth wavelength. For example, the detectors 206 on the far side of each group of emitters 204a, 204b can act as “far” detectors for detecting the light emitted by the respective group of emitters 204a, 204b, for example, light of the fourth wavelength and another wavelength, and attenuated by tissue to provide an indication of the wearer's hydration status

The physiological parameter measurement module 600 illustrates an example configuration for hydration monitoring of the wearer, with the inner group 606c of detectors 606 acting as the “near” detectors and the outer groups 606a, 606b of the detectors acting as the “far” detectors.

In the above-described configurations, each detector or detector region can provide two measurements calculated from the signals received from the closer emitter group and the signals from the further emitter group respectively. Signals detected at the far detectors can provide indication of the hydration status of the wearer as light travels through a deeper portion of the tissue of the wearer to reach the far detectors than to reach the near detectors). Signals detected at the near detectors can optionally be used as reference or for comparison with the signals detected at the far detectors when the physiological parameter measurement sensor or module processor determines the wearer's hydration status. The sensor or module processor of the physiological parameter measurement module disclosed herein can compare intensity signals of the fourth wavelength and another wavelength (for example, the third wavelength or about 905 nm) that is less sensitive to changes in water from one of the “far” detectors. The module processor can focus on the DC component, or the DC bulk absorption measurement of the signals detected by the “far” detectors for hydration status monitoring. At the DC level, water can act as a light block (that is, less transmissive of light) for the fourth wavelength and as a lens or cover (that is, more transmissive of light) for the other wavelength.

Additionally and/or alternatively, any of the modules disclosed herein can monitor the wearer's hydration status by monitoring the wearer's PVI values. The module can determine a baseline PVI value of the wearer, and can output a notification that the wearer is dehydrated or hydrated based on fluctuations in the PVI value from the baseline.

The module can further combine the hydration status monitoring by the optical detectors and other sensors (such as a sweat sensor or a skin impedance sensors) in outputting a final hydration status indication of the wearer. The module can calculate an average, a weight average or otherwise of raw hydration index values calculated based on signals from the different sensors, and/or rely on the different hydration monitoring sensors for redundancy.

As a person's hydration status is not expected to change rapidly, the physiological parameter measurement module can optionally make a measurement of the hydration status less frequently than making measurements related to the wearer's pulse rate or SpO2 or other parameters. For example, the physiological parameter measurement sensor or module processor can make a measurement of hydration status every 5 minutes, or longer, and/or upon (for example, only upon) a request by the wearer, such as when the wearer presses a button (a physical button and/or a touch button on the display) on the device or otherwise instructs the device using voice commands, hand gestures, and/or the like.

### Examples of Generally Circular Physiological Parameter Measurement Modules and Examples of Wearable Devices Incorporating the Same

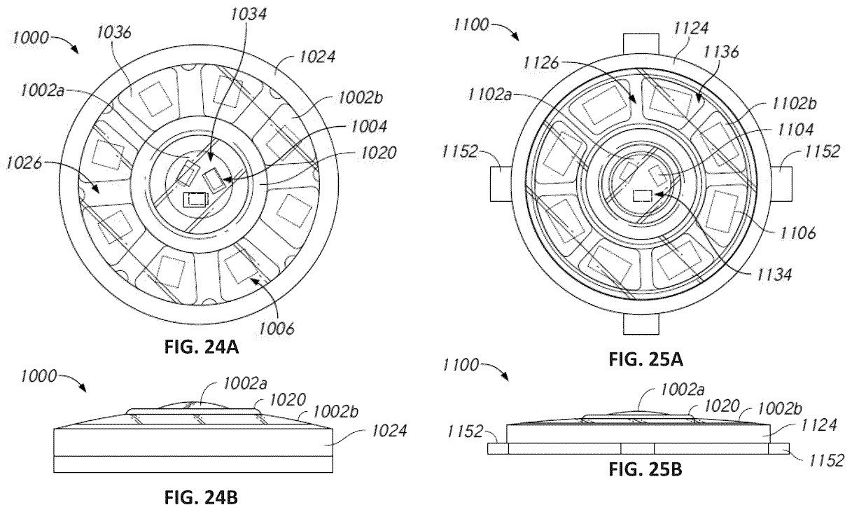

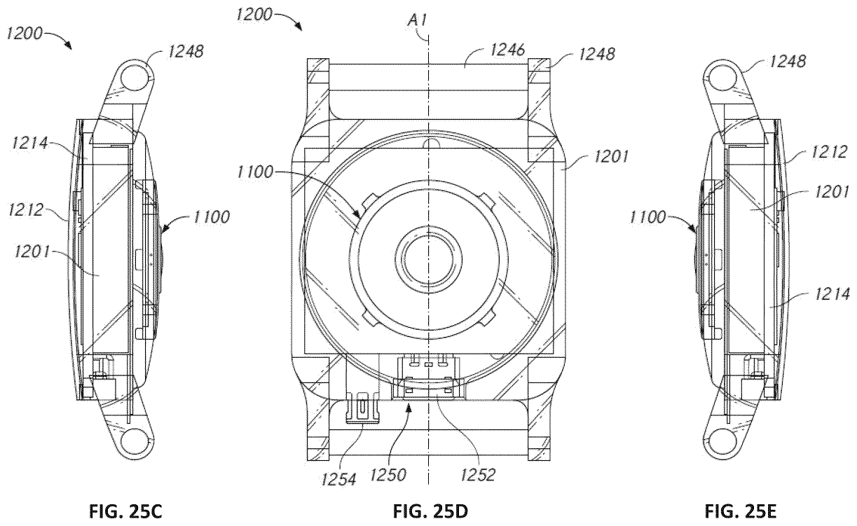

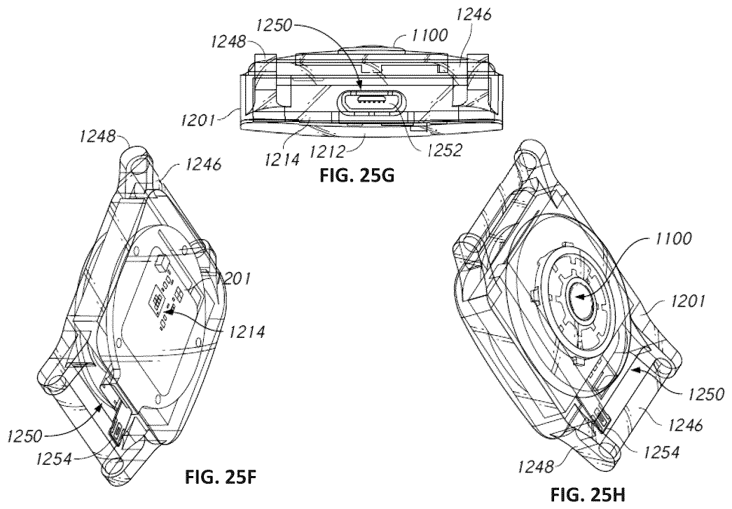

A physiological parameter measurement module can alternatively include an inner portion of emitters and an outer ring of detectors as shown in FIGS. 24A-24B and FIGS. 25A-25B. The sensor or module 1000 in FIGS. 24A-24B and the module 1100 in FIGS. 25A-25B can have any of the features of the module examples described herein, with the differences noted in the description of FIGS. 24A-24B and 25A-25B. Such a physiological parameter measurement module can have a generally circular outer shape. The sensor or module 1000 in FIGS. 24A-24B can be smaller than the module 1100 in FIGS. 25A-25B. For example, the sensor or module 1000 can have an outer diameter between about 12 mm and about 16 mm, or between about 14 mm and about 15 mm. For example, the module 1100 can have an outer diameter between about 16 mm and about 22 mm, or between about 18 mm and about 20 mm.

The physiological parameter measurement module 1000, 1100 can each include a single emitter group having a plurality of emitters 1004, 1104, such as three emitters. The emitters 1004, 1104 of the sensor or module 1000, 1100 can emit at least the first, second, and third wavelengths as described above. The emitters 1004, 1104 can be located at or near a center portion of a PCB of the sensor or module 1000, 1100. The sensor or module 1000, 1100 can include a temperature sensor located on the PCB near the emitters 1004, 1104.

The sensor or module 1000, 1100 can include a plurality of detectors 1006, 1106 (for example, about six, eight, or more) that can be arranged on the PCB so that the detectors 1006, 1106 are spaced apart around the emitters 1004, 1006. The emitters 1004, 1104 and the detectors 1006, 1106 can be separated by a first light barrier 1020, 1120. The first light barrier 1020, 1120 can surround the emitters 1004, 1104. The first light barrier 1020, 1120 can also suppress light emitted by the emitters 1004, 1104 at an angle so that the emitted light exits the inner lens or cover 1002a, 1102a in a direction generally parallel to the height of the first light barrier 1020, 1120.

The emitters 1004, 1104 can be covered by an inner lens or cover 1002a, 1102a. In the illustrated example, the inner lens or cover 1002a, 1102a can be generally circular. The detectors 1006, 1106 can be covered by an outer lens or cover 1002b, 1102b. The outer lens or cover 1002b, 1102b can be generally concentric with the inner lens or cover 1002a, 1102a. In the illustrated examples, the outer lens or cover 1002b, 1102b can be a disc when viewed directly above from the sensor or module 1000, 1100. In other examples such as those disclosed herein, the outer lens or cover can have other shapes, for example, being elliptical or otherwise. The outer lens or cover 1002b, 1102b can have a smaller curvature than the inner lens or cover 1002a, 1102a such that the inner lens or cover 1002a, 1102a protrudes more than if the inner lens or cover had the same curvature as the outer lens or cover 1002b, 1102b. As shown in FIGS. 24B and 25B, the first light barrier 1020, 1120 can protrude slightly from, that is, proud of the outer edge of the inner lens or cover 1002a, 1102a. The slightly protruding first light barrier 1020, 1120 and/or inner lens or cover 1002a, 1102a can be pressed into the wearer's skin at a higher pressure than the remainder of the light barriers or lenses or covers of the sensor or module 1000, 1100.

The detectors 1006, 1106 can be enclosed within a module side wall 1024, 1124 that defines a perimeter of the sensor or module 1000, 1100. The perimeter can be generally circular or of any other shape. The side wall 1024, 1124 can be shorter than the first light barrier 1020, 1120. The height of the side wall 1024, 1124 can be such that the tissue-facing end of the side wall 1024, 1124 is generally continuous with the curvature of outer lenses or covers 1002b, 1102b. In the illustrated example, the detectors 1006, 1106 can be separated from one another by a plurality of generally opaque divider barriers 1026, 1126. The divider barriers 1026, 1126 can have a height lower than the first light barrier 1020, 1120. The height of the divider barriers 1026, 1126 can be configured to accommodate the outer lens or cover 1002b, 1102b such that when assembled, the outer lens or cover 1002b, 1102b forms a substantially smooth surface with the module side wall 1024, 1124. The outer lens or cover 1002b, 1102b can sit on top of the divider barriers 1026, 1126. The tissue-facing end of the first light barrier 1020, 1120 and the side wall 1024, 1124, and the tissue-facing surfaces of the inner lens or cover 1002a, 1102a and the outer lens or cover 1002b, 1102b can be configured to contact the skin of the wearer and form the tissue-facing surface of the sensor or module 1000, 1100.

The first light barrier 1020, 1120, the side wall 1024, 1124, and the divider barriers 1026, 1126 can optionally form a single light barrier construct. The single light barrier construct can receive the PCB of the sensor or module 1000, 1100, and the emitters 1004, 1104, detectors 1006, 1106, temperature sensor, and any other sensors, for example, the gyroscope, the accelerometer, and/or the like, and the sensor or module processor that are located on the PCB. The single light barrier construct can receive the lenses, including the inner lens or cover 1002a, 1102a and the outer lens or cover 1002b, 1102b on another end that is opposite the end receiving the PCB. As shown in FIGS. 25A and 25B, the light barrier construct of the module 1100 or the PCB can additionally include a plurality of (for example, four or otherwise) extension prongs 1152. The plurality of extension prongs 1152 can be generally equally spaced around the side wall 1124.

The sensor or module 1000, 1100 can include a plurality of chambers such that light cannot travel between the chambers because of the various light barriers described herein. A first chamber 1034, 1134 can be enclosed by the inner lens or cover 1002a, 1102a, the first light battier 1020, 1120, and a portion of the PCB. The first chamber 1034, 1134 can enclose the emitters 1004, 1104. A plurality of second chambers 1036, 1136 can be enclosed by the outer lens or cover 1002b, 1102b, the first light barrier 1020, 1120, the divider barriers 1026, 1126, the side wall 1024, 1124, and part of the PCB. Each of the second chambers 1036, 1136 can enclose one detector 1006, 1106.

The light diffusing materials described above can be included in one or more of the chambers 1034, 1134, 1036, 1136 of the module housing to improve distribution of emitted lighted and/or detected light. The inner lens or cover 1002a, 1102a and the outer lens or cover 1002b, 1102b can also include glass microspheres as described above.

The watch 1200 in FIGS. 25C-25H is illustrated as incorporating the module 1100 shown in FIGS. 25A-25B. However, any of the example watches disclosed herein can incorporate the physiological parameter measurement module 1000, 1100 shown in FIGS. 24A-24B or FIGS. 25A-25B. The watch 1200 can have any of the features of the wearable devices disclosed herein, such as the watch 700, 900, all of which are not repeated for brevity. The watch processor 1214 and power source can be enclosed within the watching housing 1201. The watch housing 1201 can include a connection port opening 1250 configured to allow access to a connection port 1252 in electrical communication with the watch processor 1214 and/or the power source. The opening 1250 can be on one side of the watch 1200 perpendicular to the first axis A1 of the module 1100, closer to the strap coupling mechanisms. The connection port 1252 can allow for charging of the power source and/or data transfer to and from the watch processor 1214. Optionally, as shown in FIGS. 25D, 25F, and 25H, the watch 1200 can include a cable connector 845 extending outward from the watch housing 1201. The cable connector 1245 can be located adjacent to or near the connection port opening 1250.

The watch 1200 can include a display screen 1212 positioned at a first side of the watch housing 1201. The watch housing 1201 has a second side that is opposite the first side. The second side of the watch housing 1201 can include an opening sized to retain the physiological parameter measurement module 1100 while still allowing the tissue-facing surface of the module 1100 to be exposed. The extension prongs 1152 of the module 1100 can be received into corresponding structures, for example, recesses, on the second side of the watch housing 1201, which can prevent rotation of the module 1100 when being installed in the watch 1200. The second side of the watch housing 1201 can be removably attached to the first side of the watch housing 1201 without using external fasteners or via one or more fasteners as described above. An electrical connection can be established between the physiological parameter measurement module circuit and the watch circuit. Optionally, the electrical connection can include a flex circuit.

The watch housing 1201 can include strap coupling extensions 1248 on opposite sides of the watch 1200 along the first axis A1 of the module 1100. The extensions 1248 can include a bar 1246 for coupling to any suitable watch straps.

### Example Second Sensor Connection on Physiological Parameter Measurement Modules for Preventing Opioid Overdose

The physiological parameter measurement module examples disclosed herein can include an optional connector 118 (see FIG. 7A) for receiving a second sensor, which can be a plethysmograph sensor or other suitable sensors. The connector 118 can be oriented such that the second sensor can extend from a housing of the device 10 with reduced or no impingement of the tissue at the device/tissue interface, resulting in less or no effect of the connection of a second sensor to the connector 118 on the blood flow through the device measurement site. The second plethysmograph sensor can include any suitable plethysmograph sensors, for example, a fingertip sensor configured to monitor opioid overdose as described in U.S. Pub. No. 20190374173, the entirety of which is incorporated herein by reference and should be considered part of the disclosure. FIG. 1C illustrates a non-limiting example of the second sensor 119 that is a fingertip sensor. The second sensor 119 can extend from a wearable device as shown in FIG. 1C or any of the wearable device examples disclosed herein.

Alternative to the connection to a wearable device as shown in FIG. 1C, the connector from the watch disclosed herein can extend from an opening on a tissue-facing side of the device housing, for example, on a raised platform 703, 903 (FIGS. 201 and 23A). The connector can be coupled to the PCB 616 via a cable, which can optionally have a length configured to extend around the raised platform 703, 903, for example, in a groove of the raised platform 703, 903, or otherwise. Having the cable extending around the raised platform 703, 903 can allow adjustment of the slack of the cable when the connector connects to the second sensor. Having the connector extending from an opening on the raised platform 703, 903 can also avoid the connector and/or the cable impinging on the tissue at the watch/tissue interface as described above. The connector can alternatively be located at other suitable locations on the watch 700, 900.

The second plethysmograph sensor can have a higher measurements accuracy than the physiological parameter measurement module disclosed herein. The wearer can disconnect and/or deactivate the second sensor while the wearer is awake and/or moving about. The wearer can connect and activate the second sensor, for example, when going to sleep or resting. The sensor or module processor can ignore signals from the detectors of the module when the second sensor is activated so that the sensor or module processor can output physiological parameters based on the readings from the second sensor. Alternatively, the sensor or module processor can output physiological parameters based on a combination of the readings from the second sensor and the detectors of the module. The wearer can have the flexibility of choosing to use the physiological parameter measurement module and/or the second sensor, depending on the wearer's need.

The second plethysmograph sensor can aid in detection of opioid overdose in a wearer who uses opioid (for example, for medical reasons), in particular, by detecting low saturation of oxygen in the blood of the wearer. Depressed breathing is the most dangerous side effect of opioid overdose. Lack of oxygen to the brain can not only result in permanent neurologic damage, but may also be accompanied by the widespread failure of other organ systems, including the heart and kidneys. If a person experiencing an opioid overdose is left alone and asleep, the person could easily die as the respiratory depression worsens. The second plethysmograph sensor can be configured to detect depressed breathing by detecting decreased oxygen saturation in the blood of the wearer. The wearable device can be configured to automatically notify a first responder and/or the wearer's family or guardian in response to detecting opioid overdose of the wearer.

Optionally, the device processor of the wearable device can be in communication (for example, via Bluetooth or NFC communication, or via the network) with a processor of a drug delivery apparatus that is wearable by the wearer and configured to deliver one or more doses of a therapeutic drug, such as opioid. The drug delivery apparatus can include a delivery device that includes a dose of a therapeutic drug stored in a reservoir, a drug delivery channel, a dispensing device to dispense the therapeutic drug from the reservoir through the drug delivery channel, and activation circuitry to activate the dispensing device. The processor of the drug delivery apparatus can receive the parameters measured by the second plethysmograph sensor of the wearable device disclosed herein. The processor of the drug delivery apparatus can store memory-storing instructions and be configured to execute the instructions to at least compare the received parameters from the wearable device to a threshold that is indicative of opioid overdose. The processor of the drug delivery apparatus can determine whether an overdose event is occurring or likely to occur based on the comparison and send at least one activation signal to the drug delivery apparatus to dispense at least one dose of the therapeutic drug based on the determination.

Alternatively, the sensor or module processor of the physiological parameter measurement module can perform the comparison of the parameters measured by the second plethysmograph sensor to the predetermined opioid overdose threshold. Optionally, a microneedle patch may be used for providing a medication that can counteract opioid overdose. The wearer can apply the microneedle patch containing the medication to the skin when the wearable device outputs an alert that the wearer's physiological parameters (for example, SpO2) has exceeded a threshold (which may be indicative of opioid overdose).

Alternatively or additionally, the second sensor can be any other suitable noninvasive sensor disclosed herein. Alternatively or additionally, the physiological parameter measurement module examples disclosed herein can connect to a second sensor via wireless connection, for example, using Bluetooth technology. The module can receive measured parameters from the connected second sensor and/or process the sensor data received from the second sensor to calculate additional physiological parameters.

### Example Microneedle Patch

In addition and/or alternative to delivering medication to prevent opioid overdose as described herein, a microneedles patch can be used for other purposes in combination with the wearable device. Microneedles have been used in recent years as a painless alternative to hypodermic needles to deliver drugs to the body. Microneedles on a patch can be placed on an arm or leg, or other parts of the body, which then create small holes in the skin's outermost layer, allowing the drugs coated on each needle to diffuse into the body. Microneedles can be made from silicon, metals, synthetic polymers, or natural, biodegradable materials such as silk and chitin.

Because of the small size, microneedles are minimally invasive and cause less pain compared to larger needles (for example, hypodermic needles). Additionally, the microneedle patch are easier to apply by the wearer than a hypothermal needle. In comparison, larger needles may require correct injection depth and injection angle to ensure that the drugs are injected at a right location.

FIG. 26A illustrates schematically a microneedle 3100 of a microneedle patch that has penetrated the tissue surface 2 of the wearer. Depending on its height, the microneedle 3100 may have varying injection depths. For example, the microneedle 3100 may puncture just the epidermis (including the stratum corneum, which is the outer layer of the epidermis) 42. In other examples, the microneedle 102 may puncture the epidermis 42 and dermis 44, with a tip of the microneedle 3102 terminating in the dermis 44. In other examples such as shown in FIG. 26A, the microneedle 3100 may puncture the epidermis 42 and dermis 44, with the tip 3102 end in the subcutaneous tissues 46.

Depending on the use, the microneedles 3100 with different heights may be used for delivery of medication and/or irrigation fluid 3104 into different parts of the wearer's tissue. The microneedles 3100 can be used to deliver a broad range of drugs, biotherapeutics, and vaccines. The microneedles 3100 can be hollow with internal reservoirs to store and deliver drugs or irrigation fluid 3104. Alternatively, the microneedles 3100 can be solid and coated with drugs 3104, and optionally other surfactant/thickening agents. Optionally, the microneedle 3100 can be dissolvable and encapsulate the drug in a nontoxic polymer that can dissolve once inside the skin.

Alternatively or additionally, the microneedles 3100 can be used to extract a tissue fluid sample 3104 (for example, the interstitial fluid of the wearer) for detection and/or analysis of analytes in the sample 3104. Optionally, the microneedle 3100 can irrigate the tissue of the wearer with a fluid before extracting the fluid (which, for example, may have equilibrated with the chemical composition of the wearer's bodily fluid sample) back into the microneedles 3100. The microneedles 3100 can be hollow and can extract a fluid sample via surface tension. The analyte detection and/or analysis can provide information such as the hydration status, glucose concentration, hemoglobin concentration, and/or orthogonal information about the fluid. The analyte detection and/or analysis can provide additional information related to, for example, sodium, potassium, glucose, chloride, bicarbonate, blood urea nitrogen, magnesium, creatinine, LDL cholesterol, HDL cholesterol, triglyceride, pH, and the like.

A microneedle patch may be located under one of the straps or the body of the wearable device, or be applied remotely (anywhere else on the wearer's body) from the wearable device without contacting the device. A plurality of microneedle patches can be applied to the wearer at different locations on the wearer's body. As shown in FIGS. 26B and 26C, the microneedles 3100 may be connected to a patch body 3106, forming a microneedle patch 3108. The patch body 3106 may be circular, oval, rectangular, square, triangular, tear-drop shaped, or of any other shape. The size of the patch body 3106 is not limiting. A surface of the patch body 3106 that is not connected to the microneedles 3100 can include an adhesive layer for releasably attach the patch 3108 to the wearable device. The adhesive layer may be covered by a back layer, which can be peeled off before applying the patch 3108 to the wearable device.

As shown in FIG. 26B, the microneedle patch 3108 can be placed on the body of the device 10. The patch 3108 can be applied under the skin-facing surface of the physiological parameter measurement sensor or module 100. The microneedles 3100 of the microneedle patch 3108 can face the skin of the wearer of the device 10 when the device 10 is worn. Accordingly, when the device 10 is worn, for example, on the wrist of the wearer with the straps wrapped around the wearer's wrist, the microneedles 3100 can puncture the skin on the wrist.

Additionally or alternatively, the microneedle patch 3108 may be integrated or releasably secured to an inner side of the adjustable strap 30 of the wearable device 10, such as shown in FIG. 26C. The microneedles 3100 can be pointing toward the skin around the wrist of the wearer when the device 10 is worn. When the strap 30 is wrapped around the wrist of the wearer, the microneedle patch 3108 may come in contact with the skin around the wrist of the wearer and the microneedles 3100 can penetrate the skin of the wearer.

As shown in FIG. 26D, the microneedle patch 3108 can communicate with the wearable device 10, using the wearable device 10 as a platform or hub to detect and/or analyze analytes in the fluid sample collected in the microneedles patch 3108. The patch 3108 can optionally include a sensor 3110, for example, an electrochemical sensor (with electrodes built into the microneedles), a colorimetric sensor, or otherwise. Alternatively, the patch 3108 can be brought to an external sensor for analyte detection and analysis. The patch 3108 can include an antenna 3112, which may be an NFC antenna or otherwise. The sensor 3100 can output a signal via the antenna 3112. The wearable device can receive the signal from the sensor 3100 via the antenna 19. The device processor 14 (or optionally the sensor or module processor of the physiological parameter measurement sensor or module on the device 10) can process the signal from the sensor 3100 to determine the presence and/or concentration of certain analyte(s) in the fluid sample.

### Examples Device Tightness Monitoring Systems and Methods

A desired tightness and/or pressure of the device on the body can be indicated by the skin interfacing with the wearable device moving with the device when the device is moved. If there is insufficient tightness and/or pressure of the device on the body of the wearer, ambient light entering the device-skin interface can result in noises in the signals detected by the detectors, and therefore inaccurate measurements made by the device. If the device is worn too tight (and/or the pressure exerted by the device on the body is too high), blood pulsation and circulation at the wrist can be restricted, which can lead to a decrease in oxygen saturation readings of the wearer of the device. Optionally, the device can output a warning that the device is worn too tight (which can include a message displayed on the device to the wearer to loosen the straps) when the device has determined that the wearer's oxygen saturation readings are decreasing by a certain percentage, at a certain rate, and/or at a certain rate within a predetermined amount of time.

The device 10 can include an optional strain gauge 20 (see FIG. 7A) to measure a pressure of the device 10 on the wearer. The strain gauge 20 can be located in a device housing 101 between the physiological parameter measurement module 100 and other components of the device 10, for example, the power source 16, the device processor 14, or otherwise. For example, the strain gauge 20 can be flanged between the physiological parameter measurement module 100 and the device processor 14. When the device 10 is worn on the wearer, for example, on the wrist, the pressure exerted by the module, particularly by the convex protrusion of the cover 102 against the tissue can be transmitted to and measured by the strain gauge 20. The strain gauge 20 can also be incorporated in the other wearable device examples disclosed herein.

Readings from the strain gauge 20 can be communicated to the device processor 14, which can process the readings and output an indication of the pressure asserted by the device 10 on the wearer to be displayed on the display 12. The indication can be in a variety of suitable forms, for example, using different colors to indicate whether the pressure is too low, appropriate, or too high for obtaining accurate or reliable measurements using the physiological parameter measurement module 100. In one example, the device 10 can display a green light when the pressure on the wearer is suitable for using the physiological parameter measurement module 100 and display a red or other colored light for a pressure that is too high or too low than the desired pressure or pressure range. The physiological parameter measurement module 100 may not be activated unless the readings from the strain gauge 20 indicate that the desired pressure or pressure range has been achieved. Optionally, the device processor can also deactivate the physiological parameter measurement module 100, and/or any other sensors on or attached to the device 10, in response to not detecting any readings from the strain gauge 20, indicating that the device 10 is not worn on the wearer. Automatically turning on and/or off the sensors on or attached to the device 10 can reduce power consumption and increase battery life of the device 10.

Optionally, the wearable device 10 can include a motor to adjust tightness of the straps based on a monitored tightness of the straps and/or pressure exerted by the sensor or module 100 on the wearer's skin.

### Example Additional Features of the Wearable Device

The wearable device examples disclosed herein can provide protection of the wearer's safety by sending an alert to a first responder (for example, a hospital emergency room, a firefighter, 911, security at the facility where the wearer is located, or otherwise) and/or the wearer's family or guardian when the wearer is in danger, for example, when the wearer is drowning. The wearable device can include a swim mode, which the wearer can activate when going swimming. The physiological parameter measurement module of the wearable device can monitor one or more parameters to determine that the wearer is likely drowning (such as drowning of a child in water), for example, by determining that the wearer's respiratory rate has become irregular (such as showing fluctuations greater than a predetermined number per minute), or the wearer's SpO2 value declines by a predetermined amount, or otherwise. Alternatively, the module processor can determine that wearer is likely drowning based on the gyroscope and/or accelerometer readings, which can further be combined with the parameters monitored by the other sensors. In response to determining that the wearer is likely drowning, the module can send a notification to the processor of the wearable device, which can send an alert to a first responder and/or the wearer's family or guardian. Additionally or alternatively, the wearable device can include a distress button that the wearer can push in an emergency, such as when the wearer is drowning, has sustained a fall (which can alternatively or additionally be determined using the gyroscope and/or accelerometer readings, which can further be combined with the parameters monitored by the other sensors) while being alone, or otherwise.

The physiological parameters (for example but not limited to, SpO2, PR, PI, PVI, RR, Hydration, ECG-related parameters, etc.) measured by the module disclosed herein can be reliable enough for healthcare or medical purposes, for example, in hospitals. The module can be configured to take measurements at the same time every day. The wearable device (or the physiological parameter measurement module of the device) can further include a hospital patient ID tag on a near-field communication (NFC) or Bluetooth chip, or a watch strap or band. Essential patient information, such as the patient's name, admission date, reason for admission, blood type, drug allergies, etc. can be stored on the memory device of the watch or the physiological parameter measurement module. The patient ID tag cannot be easily removed and/or may include special tools like theft prevention devices, for example, requiring the patient to cut the watch strap off. Alternatively, the wearable device can display the patient information (for example, name, admission date, etc.) on the screen when the patient is admitted to the hospital. The patient ID tag can be either disposable after the patient is discharged or reusable after disinfection. The physiological parameter measurement module can be removed and replaced when the patient ID tag (for example, the watch band) is changed. If the wearable device is worn by a caregiver, the caregiver can use the wearable device for communications with other caregivers (for example, to share critical, substantially real-time information about patients, update changes in patient status, and/or the like), replacing the need for specialized communication tools, for example, Vocera®, Spok®, etc.

### Additional Example Aspects and Implementations of a Sensor or Module

FIG. 27A is a front view of an example aspect of a sensor or module 2700. The sensor or module 2700 includes an opaque frame 2726, one or more electrodes 2724, one or more detector chambers 2788, one or more emitter chambers 2778, and a light barrier construct 2720.

The opaque frame 2726 can include one or more materials configured to prevent or block the transmission of light. In some aspects, the opaque frame 2726 may form a single integrated unit. In some aspects, the opaque frame 2726 may be formed of a continuous material. The light barrier construct 2720 can include one or more materials configured to prevent or block the transmission of light. In some aspects, the light barrier construct 2720 may form a single integrated unit. In some aspects, the light barrier construct 2720 may be formed of a continuous material. In some aspects, the light barrier construct 2720 and the opaque frame 2726 may form a single integrated unit. In some aspects, the light barrier construct 2720 and the opaque frame 2726 may be separably connected.

The light barrier construct 2720 may include one or more light barriers, such as light barriers 2720a, 2720b, 2720c, 2720d, which are provided as non-limiting examples. In some aspects, light barriers may be also be referred to as light blocks herein. The light barriers may form one or more portions of the light barrier construct 2720. The light barrier construct 2720 (or light barrier portions thereof) may prevent light from passing therethrough. The light barrier construct 2720 may include spaces between various light barriers which may define one or more chambers (e.g., detector chambers 2788, emitter chambers 2778). In some aspects, the one or more chambers (e.g., detector chambers 2788, emitter chambers 2778) may be enclosed by the light barrier construct 2720 or light barrier portions thereof, a surface of a substrate (e.g., PCB), and a lens or cover. In some aspects, light may only enter the chambers through the lens or cover.

An example of a light barrier is provided with reference to example light barrier 2720a. Light barrier 2720a forms a portion of light barrier construct 2720. Light barrier 2720a may prevent (e.g., block) light from passing therethrough between adjacent chambers. For example, light barrier 2720a may prevent light from passing through the light barrier construct 2720 between an emitter chamber 2778 and a detector chamber 2788. Light barrier 2720a, or portions thereof, may include a width 2771. In some aspects, width 2771 may be less than about 1.85 mm. In some aspects, width 2771 may be less than about 1.9 mm. In some aspects, width 2771 may be less than about 1.95 mm. In some aspects, width 2771 may be about 1.88 mm. In some aspects, the width 2771 may be less (e.g., smaller) than length 2779. In some aspects, width 2771 may be less than about 55% of length 2779. In some aspects, width 2771 may be less than about 60% of length 2779. In some aspects, width 2771 may be less than about 65% of length 2779. In some aspects, width 2771 may be about 58.9% of length 2779.

Another example of a light barrier is provided with reference to example light barrier 2720b. Light barrier 2720b forms a portion of light barrier construct 2720. Light barrier 2720b may prevent (e.g., block) light from passing therethrough between adjacent chambers. For example, light barrier 2720b may prevent light from passing through the light barrier construct 2720 between an emitter chamber 2778 and a detector chamber 2788. Light barrier 2720b, or portions thereof, may include a width 2772. In some aspects, width 2772 may be less than about 1.35 mm. In some aspects, width 2772 may be less than about 1.40 mm. In some aspects, width 2772 may be less than about 1.45 mm. In some aspects, width 2772 may be about 1.37 mm. In some aspects, the width 2772 may be substantially similar to width 2771. In some aspects, the width 2772 may be less (e.g., smaller) than width 2771. In some aspects, width 2772 may be less than about 70% of width 2771. In some aspects, width 2772 may be less than about 75% of width 2771. In some aspects, width 2772 may be less than about 80% of width 2771. In some aspects, width 2772 may be about 72.9% of width 2771.

Another example of a light barrier is provided with reference to example light barrier 2720c. Light barrier 2720c forms a portion of light barrier construct 2720. Light barrier 2720c may prevent (e.g., block) light from passing therethrough between adjacent chambers. For example, light barrier 2720c may prevent light from passing through the light barrier construct 2720 between adjacent detector chamber 2788.

Another example of a light barrier is provided with reference to example light barrier 2720d. Light barrier 2720d forms a portion of light barrier construct 2720. Light barrier 2720d may prevent (e.g., block) light from passing therethrough between adjacent chambers. For example, light barrier 2720d may prevent light from passing through the light barrier construct 2720 between adjacent emitter chambers 2778. In some aspects, light barrier 2720d may have a width 2775 separating adjacent emitter chambers of less than about 1.30 mm. In some aspects, width 2775 may be less than about 1.25 mm. In some aspects, width 2775 may be less than about 1.20 mm. In some aspects, width 2775 may be about 1.20 mm. In some aspects, width 2775 may be substantially similar to width 2772. In some aspects, width 2775 may be less (e.g., smaller) than width 2772. In some aspects, width 2775 may be less than about 95% of width 2772. In some aspects, width 2775 may be less than about 90% of width 2772. In some aspects, width 2775 may be less than about 85% of width 2772. In some aspects, width 2775 may be about 87.6% of width 2772.

The emitter chambers 2778 are positioned within a central region of the sensor or module 2700. The emitter chambers 2778 may be positioned adjacent to one another across a centerline of the sensor or module 2700 as described in greater detail with reference to FIG. 27K, for example. The emitter chambers 2778 may be positioned adjacent the center point C₁. Each of the emitter chambers 2778 may be a similar size and/or shape. The emitter chambers 2778 may be separated, at least in part, by light barrier 2720d of the light barrier construct 2720. In some aspects, as shown in this example, the light barrier 2720d may form an entire distance between emitter chambers 2778. For example, emitter chambers 2778 may be separated by only the light barrier 2720d such that other components (e.g., detectors, detector chambers, etc.) are not positioned between the emitter chambers 2778.

A portion of the emitter chambers 2778 may extend a length 2779 away from center point C₁. In some aspects, length 2779 may be less than about 3.15 mm. In some aspects, length 2779 may be less than about 3.20 mm. In some aspects, length 2779 may be less than about 3.25 mm. In some aspects, length 2779 may be about 3.19 mm. In some aspects, the length 2779 may be greater (e.g., larger) than a width of a light barrier separating an emitter chamber from a detector chamber such as width 2771. In some aspects, length 2779 may be greater than about 165% of width 2771. In some aspects, length 2779 may be greater than about 170% of width 2771. In some aspects, length 2779 may be greater than about 175% of width 2771. In some aspects, length 2779 may be about 169.7% of width 2771.

As shown in this example aspect, the detector chambers 2788 are arranged in a substantially circular pattern. Each of the detector chambers 2788 houses a detector 2706 positioned on a substrate (e.g., PCB) in a substantially circular or annular pattern. The detectors 2706 may be positioned in a central region of each of the respective detector chambers 2778. The detector chambers 2788 are arranged along a ring defined by ring L₁. In some aspects, such as shown in this example aspect, detectors 2706 of respective detector chambers 2788 may also be arranged along a same ring along which the detector chambers 2788 are arranged (such as in aspects where detectors are positioned in a central region of respective chambers). The ring L₁ may intersect a central region of the detector chambers 2788. In this example aspect, the ring L₁ encloses an entirety of the emitter chambers emitter chambers 2778 such that the emitter chambers 2778 are positioned within an interior region (e.g., a central region) of the ring L₁ defined by the detector chambers 2788. In some aspects, each of the detector chambers 2788 (and corresponding detectors 2706 within respective detector chambers 2788) may be positioned at a substantially similar or same distance away from the center point C₁ (e.g., center of sensor or module 2700). In some aspects, the detectors 2706 may be rectangular including longer sides and shorter sides. The detectors 2706 may be positioned on a substrate of the sensor or module 2700 such that a long side of each detector is orthogonal to a radius extending away from center point C₁ (e.g., radius r₁, radius r₂, radius r₃). Advantageously, orienting the detectors 2706 on the sensor or module 2700 in an annular arrangement with a long side of the detectors 2706 orthogonal the center point C₁ may improve an accuracy of physiological measurements by ensuring that light from emitters travels along a known path length from emitters to the detectors 2706 and may also reduce processing requirements of the sensor or module 2700 by reducing the amount of variables (e.g., number of light path lengths) required to process in order to determine physiological data.

The electrodes 2724 can include a reference electrode and a negative electrode (and/or a positive electrode). In some aspects, a wearable device such as a watch incorporating the sensor or module 2700 can include another ECG electrode (e.g., a positive electrode) located on the housing of the wearable device configured to make contact with the wearer's skin. In some configurations, a surface of the electrodes 2724 may be flush with a surface of the opaque frame 2726.

The electrodes 2724 are positioned within or along a portion of the opaque frame 2726 such as shown in FIG. 27B for example. In some aspects, the electrodes 2724 can be substantially semicircular. In some aspects, the electrodes 2724 can be substantially semiannular. In the example aspect shown, each of the electrodes 2724 forms a substantial half annulus. Advantageously, an annular shaped electrode may improve contact with the skin of a wearer (e.g., by contacting a diverse area of skin) while simultaneously reducing the amount of surface area of the electrode. In some aspects, each of the electrodes 2724 may be a similar size and/or shape. In some aspects, the electrodes 2724 may be various sizes and/or shapes. In this example aspect, the electrodes 2724 are positioned within the sensor or module 2700 (e.g., within the opaque frame 2726) along ring defined by L₂. In various aspects described herein, the ring L₂ may include various radii which may advantageously provide improved contact between the electrodes 2724 and the skin of a wearer of the device.

The opaque frame 2726 includes one or more gaps (e.g., g₁, g₂) between electrodes 2724. The gaps g₁, g₂, (or other portions of the opaque frame 2726) may electrically insulate each of the electrodes 2724 from one another. Each of the electrodes 2724 includes substantially straight edge along a portion of respective gaps g₁, g₂. In some aspects, the gaps g₁, g₂, may be a similar or a same size. In some aspects, the gaps g₁, g₂, may be a different size than each other. In some aspects, the gaps g₁, g₂, may be less than about 1.6 mm. In some aspects, the gaps g₁, g₂, may be less than about 1.65 mm. In some aspects, the gaps g₁, g₂, may be less than about 1.7 mm. In some aspects, the gaps g₁, g₂, may be about 1.62 mm. As discussed above, in some implementations the frame 2726 includes recesses 2824 sized and/or shaped to receive the ECG electrodes 2724. In some implementations, each of such recesses 2824 includes first and second ends, the first ends of the recesses 2824 are separated from one another by gap g₁, and the second ends of the recesses 2824 are separated from one another by gap g₂.

The ring L₁ may be concentric with an outer perimeter of the sensor or module 2700. The ring L₂ may be concentric with an outer perimeter of the sensor or module 2700. The ring L₂ may be concentric with a ring defined by positions of the detector chambers 2788 such as ring L₁. Center point C₁ may define a geometric center of ring L₁. Center point C₁ may define a geometric center of ring L₂. Center point C₁ may define a geometric center of an outer perimeter of the sensor or module 2700. In some aspects, such as shown in FIG. 27A, each of L₁, L₂, and an outer perimeter of the sensor or module 2700 are concentric with each other and share a same geometric center shown as C₁.

The ring L₁ may include a radius r₁. In some aspects, radius r₁ may be less than about 6.25 mm. In some aspects, radius r₁ may be less than about 6.50 mm. In some aspects, radius r₁ may be less than about 6.75 mm. In some aspects, radius r₁ may be about 6.34 mm. In some aspects, the radius r₁ may be less (e.g., smaller) than radius r₂. In some aspects, radius r₁ may be less than about 55% of r₂. In some aspects, radius r₁ may be less than about 60% of r₂. In some aspects, radius r₁ may be less than about 65% of r₂. In some aspects, radius r₁ may be about 59% of r₂. In some aspects, the radius r₁ may be less (e.g., smaller) than radius r₃. In some aspects, radius r₁ may be less than about 40% of r₃. In some aspects, radius r₁ may be less than about 45% of r₃. In some aspects, radius r₁ may be less than about 50% of r₃. In some aspects, radius r₁ may be about 41.7% of r₃.

The ring L₂ may include a radius r₂. In some aspects, radius r₂ may be less than about 10.5 mm. In some aspects, radius r₂ may be less than about 10.75 mm. In some aspects, radius r₂ may be less than about 11.0 mm. In some aspects, radius r₂ may be about 10.73 mm. In some aspects, the radius r₂ may be less (e.g., smaller) than radius r₃. In some aspects, radius r₂ may be less than about 65% of r₃. In some aspects, radius r₂ may be less than about 70% of r₃. In some aspects, radius r₂ may be less than about 75% of r₃. In some aspects, radius r₂ may be about 70.6% of r₃.

In some aspects, the sensor or module 2700 (e.g., an outer perimeter of the sensor or module 2700) may include a radius r₃. In some aspects, radius r₃ may be less than about 14.5 mm. In some aspects, radius r₃ may be less than about 15.0 mm. In some aspects, radius r₃ may be less than about 15.50 mm. In some aspects, radius r₃ may be less than about 16.0 mm. In some aspects, radius r₃ may be about 15.19 mm.

FIGS. 27K-27L illustrate an example physiological parameter measurement sensor or module 2700′ and example light paths between emitters and detectors of the module 2700′.

FIG. 27K illustrates an example arrangement of emitter and detector chambers of the sensor or module 2700′. As shown, the sensor or module 2700′ can include a first emitter chamber 2736a′ enclosing a first emitter group comprising one or more emitters, a second emitter chamber 2736b′ enclosing a second emitter group comprising one or more emitters, one or more first detector chambers 2740′, one or more second detector chambers 2742′, and one or more third detector chambers 2738′. In some aspects, each detector chamber may enclose one detector.

The first emitter group of the first emitter chamber 2736a′ may comprise the same number and type of emitters as the second emitter group of the second emitter chamber 2736b′. In other words, each emitter of the first emitter group may correspond to an emitter of the same type (e.g., same wavelength) of the second emitter group. The emitters of the first emitter group may be arranged in a configuration that mirrors the emitters of the second emitter group across a centerline 2750 of the sensor or module 2700′ as shown in FIG. 27K. For example, each emitter of the first group of emitters may be located a distance away from a centerline 2750 of the sensor or module 2700′ that is a same distance that a corresponding emitter of the second group of emitters is located away from the centerline 2750 of the sensor or module 2700′. For example, the first and second emitter groups may each include an emitter that emits light of a first wavelength and that are positioned at locations that are mirror images of each other across a centerline 2750 of the sensor or module 2700′. Additionally, the first and second emitter groups may each include an emitter that emits light of a second wavelength and that are positioned at locations that are mirror images of each other across a centerline 2750 of the sensor or module 2700′. Each of the emitters of the first emitter group may correspond to an emitter of the second emitter group located at a mirror image position, and vice versa.

The one or more second detector chambers 2742′ may be bisected by a centerline 2750 of the sensor or module 2700′. Each of the detectors of the respective one or more second detector chambers 2742′ may be bisected by a centerline 2750 of the sensor or module 2700′. In other words, the one or more second detector chambers 2742′ and the respective detectors and the sensor or module 2700′ may each share a same (e.g., parallel) centerline 2750. The sensor or module 2700′ may be oriented (e.g., rotated) with respect to the tissue of a wearer in any orientation. In an example implementation where the sensor or module 2700′ is worn on a wrist of a user, the sensor or module 2700′ may be rotated in any direction with respect to the wrist or forearm of the wearer. In one example configuration, the sensor or module 2700′ may be oriented with respect to the forearm (or other body part) of a wearer such that the centerline 2750′ of the sensor or module is perpendicular to a line extending along a length of the forearm of the wearer (e.g., from the elbow to the wrist). Advantageously, such a configuration may improve physiological measurements by facilitating light emitted from the emitter chambers and detected at the detector chambers (e.g., light travelling from emitter chamber 2736a′ to detector chamber 2738′) to penetrate into soft tissue of the wearer (e.g., blood vessels) rather than other tissues such as bone. In another example configuration, the sensor or module 2700′ may be oriented with respect to the forearm (or other body part) of a wearer such that the centerline 2750′ of the sensor or module is parallel to a line extending along a length of the forearm of the wearer (e.g., from the elbow to the wrist). Advantageously, such a configuration may improve physiological measurements by facilitating light emitted from the emitter chambers and detected at the detector chambers (e.g., light travelling from emitter chamber 2736a′ to detector chamber 2742′) to penetrate into soft tissue of the wearer (e.g., blood vessels) rather than other tissues such as bone.

As shown in FIG. 27K, emitters of the first and second emitter groups that correspond to each other (e.g., emit the same wavelength and mirror each other) may each emit light that travels along respective paths to the detectors of the one or more second detector chambers 2742′. The respective paths of light from the corresponding emitters may be of equal length. This may be because the corresponding emitters are each positioned an equal distance away from a detector of a chamber 2742′. The corresponding emitters may each be an equal distance away from a detector of a chamber 2742′ because they are positioned at mirror images of each other across a centerline 2750 of the sensor or module 2700′ that bisects the one or more second detector chambers 2472′ and respective detectors.

The one or more second detector chambers 2742′ and their respective detectors may be used, at least in part, for calibration, for example to characterize the emitters, by providing known information such as a known ratio. For example, information corresponding to a wavelength detected at a detector of a chamber 2742′ from an emitter of the first group of emitters may be similar or the same as information information corresponding to that wavelength detected at the detector of the chamber 2742′ from an emitter of the second group of emitters and a comparison (e.g., subtracting, dividing, etc.) of the information resulting from the first and second groups of emitters may yield a known number such as zero or one because the corresponding emitters from the first and second emitter groups may be an equal distance from the detector of chamber 2742′ and light emitted therefrom may travel a same distance to the detector of chamber 2742′. As an example of normalization, ratios of wavelengths detected at detectors of chambers 2738′, 2740′ may be normalized (e.g., divided by) ratios of wavelengths detected at detectors of chambers 2742′. In instances where the information resulting from detection of light from the first and second groups of emitters is not the same or is substantially different (e.g., as a result of emission intensity variations or other such discrepancies) the information may be adjusted or normalized (e.g., calibrated) to account for such differences. This normalization or on-board calibration or characterization of the emitters may improve accuracy of the physiological measurements and provide for continuous calibration or normalization during measurements. In some aspects, a processor may be configured to calibrate or normalize the physiological parameter measurement of the sensor continuously. In some aspects, a processor may be configured to calibrate or normalize the physiological parameter measurement of the sensor while the optical physiological sensor measures physiological parameters of the wearer.

FIG. 27L illustrates an example arrangement of emitter and detector chambers of the sensor or module 2700′. As shown, the sensor or module 2700′ can include a first emitter chamber 2736a′, a second emitter chamber 2736b′, one or more first detector chambers 2740′, one or more second detector chambers 2742′, and one or more third detector chambers 2738′, for example as discussed elsewhere herein.

The first and second emitter chambers 2736a′, 2736b′ may be located at non-equal distances away from each of the chambers of the one or more detector chambers 2738′, 2740′. Thus, with respect to each detector chamber of the chambers 2738′, 2740′, the first and second emitter chamber 2736a′, 2736b′, may each be a “near” or “far” emitter chamber. In other words, each detector of the detector chambers 2738′, 2740′ may detect light, of any given wavelength, from both a “near” emitter and a “far” emitter, with the near and far emitters being included in either the first or second emitter group, respectively.

As an example, as shown in FIG. 27L, light of a given wavelength may travel along a path from an emitter in the first emitter group to the detector of detector chamber 2738′ and light of the same wavelength may travel along a path from an emitter in the second emitter group to the same detector. The light from the first emitter group may travel along a longer path than light from the second emitter group before reaching the detector of chamber 2738′. Thus, for any detector of detector chambers 2738′ or 2740′, the detector may receive light of a given wavelength from both a near (e.g., proximal) emitter and a far (e.g., distal) emitter. This may not be the case for detectors of chambers 2742′ because the first and second emitter groups may each be located a same distance away from any given detector of detector chambers 2742′, as described herein.

For convenience, the terms “proximal” and “distal” may be used herein to describe structures relative to any of the detector chambers or their respective detectors. For example, an emitter may be proximal to a detector chamber of the first detector chambers and distal to a detector of the second detector chambers. The term “distal” refers to one or more emitters that are farther away from a detector chamber than at least some of the other emitters. The term “proximal” refers to one or more emitters that are closer to a detector chamber than at least some of the other emitters. The term “proximal emitter” may be used interchangeably with “near emitter” and the term “distal emitter” may be used interchangeably with “far emitter”.

A single emitter may be both proximal to one detector and distal to another detector. For example, an emitter may be a proximal emitter relative to a detector of the first detector chambers and may be a distal emitter relative to a detector of the second detector chambers.

Light of a given wavelength that is detected at a detector may provide different information depending on the length of the path it has travelled from the emitter (e.g., along a long path from a distal emitter or along a short path from a proximal emitter). For example, light that has travelled along a long path from a distal emitter may penetrate deeper into the tissue of a wearer of the device and may provide information pertaining to pulsatile blood flow or constituents. The use of a proximal and distal emitter for each wavelength may improve accuracy of the measurement, for example information pertaining to light that has travelled along a long path from a distal emitter may be normalized by (e.g., divided by) information pertaining to light that has travelled along a short path from a proximal emitter.

FIGS. 27M-27P illustrate an example physiological parameter measurement sensor or module 2700′ and example light barriers or light blocks between emitter and detector chambers of the module 2700′.

FIG. 27M is a front view of an example aspect of a sensor or module 2700′. The sensor or module 2700′ includes an opaque frame opaque frame 2726′, one or more electrodes 2724′, one or more detector chambers 2788′, one or more emitter chambers 2778′, and a light barrier construct 2720′.

The opaque frame 2726′ can include one or more materials configured to prevent or block the transmission of light. In some aspects, the opaque frame 2726′ may form a single integrated unit. In some aspects, the opaque frame 2726′ may be formed of a continuous material. The light barrier construct 2720′ can include one or more materials configured to prevent or block the transmission of light. In some aspects, the light barrier construct 2720′ may form a single integrated unit. In some aspects, the light barrier construct 2720′ may be formed of a continuous material. In some aspects, the light barrier construct 2720′ and the opaque frame 2726′ may form a single integrated unit. In some aspects, the light barrier construct 2720′ and the opaque frame 2726′ may be separably connected.

The light barrier construct 2720′ may include one or more light barriers, such as light barriers 2720a′, 2720b′, 2720c′, 2720d′, which are provided as non-limiting examples. In some aspects, light barriers may be also be referred to as light blocks herein. The light barriers may form one or more portions of the light barrier construct 2720′. The light barrier construct 2720′ (or light barrier portions thereof) may prevent light from passing therethrough. The light barrier construct 2720′ may include spaces between various light barriers which may define one or more chambers (e.g., detector chambers 2788′, emitter chambers 2778′). In some aspects, the one or more chambers (e.g., detector chambers 2788′, emitter chambers 2778′) may be enclosed by the light barrier construct 2720′ or light barrier portions thereof, a surface of a substrate (e.g., PCB), and a lens or cover. In some aspects, light may only enter the chambers through the lens or cover.

An example of a light barrier is provided with reference to example light barrier 2720a′. Light barrier 2720a′ forms a portion of light barrier construct 2720′. Light barrier 2720a′ may prevent (e.g., block) light from passing therethrough between adjacent chambers. For example, light barrier 2720a′ may prevent light from passing through the light barrier construct 2720′ between an emitter chamber 2778′ and a detector chamber 2788′. Light barrier 2720a′, or portions thereof, may include a width 2771′. In some aspects, width 2771′ may be less than about 3.30 mm. In some aspects, width 2771′ may be less than about 3.25 mm. In some aspects, width 2771′ may be less than about 3.20 mm. In some aspects, width 2771′ may be about 3.24 mm. In some aspects, the width 2771′ may be greater (e.g., larger) than length 2779. In some aspects, width 2771′ may be less than about 165% of length 2779. In some aspects, width 2771′ may be less than about 160% of length 2779. In some aspects, width 2771′ may be less than about 155% of length 2779. In some aspects, width 2771′ may be about 160% of length 2779. Advantageously, a greater width 2771′ (e.g., a wider light barrier separating the emitter chambers 2778′ and detector chambers 2788′) may cause light emitted from the emitter chambers 2778′ to travel a greater distance before reaching the detector chambers 2788′. Light that travels a greater distance may penetrate deeper into the tissue of the wearer which may improve accuracy of a physiological measurement.

Another example of a light barrier is provided with reference to example light barrier 2720b′. Light barrier 2720b′ forms a portion of light barrier construct 2720′. Light barrier 2720b′ may prevent (e.g., block) light from passing therethrough between adjacent chambers. For example, light barrier 2720b′ may prevent light from passing through the light barrier construct 2720′ between an emitter chamber 2778′ and a detector chamber 2788′. Light barrier 2720b′, or portions thereof, may include a width 2772′. In some aspects, width 2772′ may be less than about 1.65 mm. In some aspects, width 2772′ may be less than about 1.60 mm. In some aspects, width 2772′ may be less than about 1.55 mm. In some aspects, width 2772′ may be about 1.59 mm. In some aspects, the width 2772′ may be less (e.g., smaller) than width 2771′. In some aspects, width 2772′ may be less than about 60% of width 2771′. In some aspects, width 2772′ may be less than about 55% of width 2771′. In some aspects, width 2772′ may be less than about 50% of width 2771′. In some aspects, width 2772′ may be about 49% of width 2771′. Advantageously, a greater width 2772′ may cause light emitted from the emitter chambers 2778′ to travel a greater distance before reaching the detector chambers 2788′. Light that travels a greater distance may penetrate deeper into the tissue of the wearer which may improve accuracy of a physiological measurement

Another example of a light barrier is provided with reference to example light barrier 2720c′. Light barrier 2720c′ forms a portion of light barrier construct 2720′. Light barrier 2720c′ may prevent (e.g., block) light from passing therethrough between adjacent chambers. For example, light barrier 2720c′ may prevent light from passing through the light barrier construct 2720′ between adjacent detector chamber 2788′.

Another example of a light barrier is provided with reference to example light barrier 2720d′. Light barrier 2720d′ forms a portion of light barrier construct 2720′. Light barrier 2720d′ may prevent (e.g., block) light from passing therethrough between adjacent chambers. For example, light barrier 2720d′ may prevent light from passing through the light barrier construct 2720′ between adjacent emitter chambers 2778′. In some aspects, light barrier 2720d′ may have a width 2775′ separating adjacent emitter chambers of less than about 1.40 mm. In some aspects, width 2775′ may be less than about 1.35 mm. In some aspects, width 2775′ may be less than about 1.30 mm. In some aspects, width 2775′ may be about 1.28 mm. In some aspects, width 2775′ may be less (e.g., smaller) than width 2771′. In some aspects, width 2775′ may be less than about 50% of width 2771′. In some aspects, width 2775′ may be less than about 45% of width 2771′. In some aspects, width 2775′ may be less than about 40% of width 2771′. In some aspects, width 2775′ may be less than about 35% of width 2771′. In some aspects, width 2775′ may be about 39.5% of width 2771′.

The emitter chambers 2778′ are positioned within a central region of the sensor or module 2700′. The emitter chambers 2778′ may be positioned adjacent to one another across a centerline of the sensor or module 2700′ as described in greater detail with reference to FIG. 27K, for example. The emitter chambers 2778′ may be positioned adjacent the center point C′₁. Each of the emitter chambers 2778′ may be a similar size and/or shape. The emitter chambers 2778′ may be separated, at least in part, by light barrier 2720d′ of the light barrier construct 2720′. In some aspects, as shown in this example, the light barrier 2720d′ may form an entire distance between emitter chambers 2778′. For example, emitter chambers 2778′ may be separated by only the light barrier 2720d′ such that other components (e.g., detectors, detector chambers, etc.) are not positioned between the emitter chambers 2778′.

A portion of the emitter chambers 2778′ may extend a length 2779 away from center point C′₁. In some aspects, length 2779 may be less than about 2.15 mm. In some aspects, length 2779 may be less than about 2.10 mm. In some aspects, length 2779 may be less than about 2.05 mm. In some aspects, length 2779 may be less than about 2.0 mm. In some aspects, length 2779 may be about 2.02 mm. In some aspects, the length 2779 may be less (e.g., smaller) than a width of a light barrier separating an emitter chamber from a detector chamber such as width 2771′. In some aspects, length 2779 may be less than about 70% of width 2771′. In some aspects, length 2779 may be less than about 65% of width 2771′. In some aspects, length 2779 may be less than about 60% of width 2771′. In some aspects, length 2779 may be about 62.3% of width 2771′.

As shown in this example aspect, the detector chambers 2788′ are arranged in a substantially circular pattern. Each of the detector chambers 2788′ houses a detector 2706 positioned on a substrate (e.g., PCB) in a substantially circular or annular pattern. The detectors 2706 may be positioned in a central region of each of the respective detector chambers 2778′. The detector chambers 2788′ are arranged along a ring defined by ring L′₁. In some aspects, such as shown in this example aspect, detectors 2706 of respective detector chambers 2788′ may also be arranged along a same ring along which the detector chambers 2788′ are arranged (such as in aspects where detectors are positioned in a central region of respective chambers). The ring L′₁ may intersect a central region of the detector chambers 2788′. In this example aspect, the ring L′₁ encloses an entirety of the emitter chambers emitter chambers 2778′ such that the emitter chambers 2778′ are positioned within an interior region (e.g., a central region) of the ring L′₁ defined by the detector chambers 2788′. In some aspects, each of the detector chambers 2788′ (and corresponding detectors 2706 within respective detector chambers 2788′) may be positioned at a substantially similar or same distance away from the center point C′₁ (e.g., center of sensor or module 2700′). In some aspects, the detectors 2706 may be rectangular including longer sides and shorter sides. The detectors 2706 may be positioned on a substrate of the sensor or module 2700′ such that a long side of each detector is orthogonal to a radius extending away from center point C′₁ (e.g., radius r′₁, radius r′₂, radius r′₃). Advantageously, orienting the detectors 2706 on the sensor or module 2700′ in an annular arrangement with a long side of the detectors 2706 orthogonal the center point C′₁ may improve an accuracy of physiological measurements by ensuring that light from emitters travels along a known path length from emitters to the detectors 2706 and may also reduce processing requirements of the sensor or module 2700′ by reducing the amount of variables (e.g., number of light path lengths) required to process in order to determine physiological data.

The electrodes 2724′ can include a reference electrode and a negative electrode (and/or a positive electrode). In some aspects, a wearable device such as a watch incorporating the sensor or module 2700′ can include another ECG electrode (e.g., a positive electrode) located on the housing of the wearable device configured to make contact with the wearer's skin. In some configurations, a surface of the electrodes 2724′ may be flush with a surface of the opaque frame 2726′.

The electrodes 2724′ are positioned within or along a portion of the opaque frame 2726′ such as shown in FIG. 27B for example. In some aspects, the electrodes 2724′ can be substantially semicircular. In some aspects, the electrodes 2724′ can be substantially semi-annular. In the example aspect shown, each of the electrodes 2724′ forms a substantial half annulus. Advantageously, an annular shaped electrode may improve contact with the skin of a wearer (e.g., by contacting a diverse area of skin) while simultaneously reducing the amount of surface area of the electrode. In some aspects, each of the electrodes 2724′ may be a similar size and/or shape. In some aspects, the electrodes 2724′ may be various sizes and/or shapes. In this example aspect, the electrodes 2724′ are positioned within the sensor or module 2700′ (e.g., within the opaque frame 2726′) along ring defined by L′₂. In various aspects described herein, the ring L′₂ may include various radii which may advantageously provide improved contact between the electrodes 2724′ and the skin of a wearer of the device. In some implementations, frame 2726′ includes recesses that 2824′ are sized and/or shaped to accommodate the ECG electrodes 2724′. In some implementations, recesses 2824′ have a depth (for example, measured from a plane of the frame 2726′) that is substantially equal to a thickness of the ECG electrodes 2724′. In some implementations, recesses 2824′ have a size and/or shape that matches a size and/or shape of the ECG electrodes 2724′. For example, in some implementations in which the ECG electrodes have a semi-annular shape, the recesses 2824′ can have a semi-annular shape.

The opaque frame 2726′ includes one or more gaps (e.g., g′₁, g′₂) between electrodes 2724′. The gaps g′₁, g′₂, (or other portions of the opaque frame 2726′) may electrically insulate each of the electrodes 2724′ from one another. Each of the electrodes 2724′ includes a curved edge along a portion of respective gaps g′₁, g′₂. In some aspects, the gaps g′₁, g′₂, may be a similar or a same size. In some aspects, the gaps g′₁, g′₂, may be a different size than each other. In some aspects, the gaps g′₁, g′₂, may be less than about 0.6 mm. In some aspects, the gaps g′₁, g′₂, may be less than about 0.65 mm. In some aspects, the gaps g′₁, g′₂, may be less than about 0.7 mm. In some aspects, the gaps g′₁, g′₂, may be about 0.62 mm. As discussed above, in some implementations the frame 2726′ includes recesses 2824′ sized and/or shaped to receive the ECG electrodes 2724′. In some implementations, each of such recesses 2824′ includes first and second ends, the first ends of the recesses 2824′ are separated from one another by gap g′₁, and the second ends of the recesses 2824′ are separated from one another by gap g′₂ (see FIG. 27M). In some implementations, such as that illustrated in at least FIG. 27M, ends of the recesses 2824′ and/or ends of ECG electrodes 2724′ have a rounded shape.

The ring L′₁ may be concentric with an outer perimeter of the sensor or module 2700′. The ring L′₂ may be concentric with an outer perimeter of the sensor or module 2700′. The ring L′₂ may be concentric with a ring defined by positions of the detector chambers 2788′ such as ring L′₁. Center point C′₁ may define a geometric center of ring L′₁. Center point C′₁ may define a geometric center of ring L′₂. Center point C′₁ may define a geometric center of an outer perimeter of the sensor or module 2700′. In some aspects, such as shown in FIG. 27M, each of L′₁, L′₂, and an outer perimeter of the sensor or module 2700′ are concentric with each other and share a same geometric center shown as C′₁.

The ring L′₁ may include a radius r′₁. In some aspects, radius r′₁ may be less than about 6.5 mm. In some aspects, radius r′₁ may be less than about 6.45 mm. In some aspects, radius r′₁ may be less than about 6.40 mm. In some aspects, radius r′₁ may be about 6.40 mm. In some aspects, the radius r′₁ may be less (e.g., smaller) than radius r′₂. In some aspects, radius r′₁ may be less than about 60% of r′₂. In some aspects, radius r′₁ may be less than about 55% of r′₂. In some aspects, radius r′₁ may be less than about 50% of r′₂. In some aspects, radius r′₁ may be about 50.9% of r′₂. In some aspects, the radius r′₁ may be less (e.g., smaller) than radius r′₃. In some aspects, radius r′₁ may be less than about 40% of r′₃. In some aspects, radius r′₁ may be less than about 45% of r′₃. In some aspects, radius r′₁ may be less than about 50% of r′₃. In some aspects, radius r′₁ may be about 42% of r′₃.

The ring L′₂ may include a radius r′₂. In some aspects, radius r′₂ may be less than about 13 mm. In some aspects, radius r′₂ may be less than about 12.75 mm. In some aspects, radius r′₂ may be less than about 12.5 mm. In some aspects, radius r′₂ may be about 12.59 mm. In some aspects, the radius r′₂ may be less (e.g., smaller) than radius r′₃. In some aspects, radius r′₂ may be less than about 80% of r′₃. In some aspects, radius r′₂ may be less than about 85% of r′₃. In some aspects, radius r′₂ may be less than about 90% of r′₃. In some aspects, radius r′₂ may be about 82.7% of r′₃.

In some aspects, the sensor or module 2700′ (e.g., an outer perimeter of the sensor or module 2700′) may include a radius r′₃. In some aspects, radius r′₃ may be less than about 15 mm. In some aspects, radius r′₃ may be less than about 15.0 mm. In some aspects, radius r′₃ may be less than about 15.25 mm. In some aspects, radius r′₃ may be less than about 15.5 mm. In some aspects, radius r′₃ may be about 15.22 mm.

FIG. 27N is a side cutaway view of an example aspect of a sensor or module 2700′. The sensor or module 2700′ includes a barrier construct 2720′, an outer surface 2791′, and a substrate 2716′. The outer surface 2791′ may include light barrier construct portions, lens portions, opaque frame portions, and/or electrode portions. The outer surface 2791′ of the sensor or module 2700′ may face and/or contact the skin of a wearer and may include a generally convex shape. A central region of the sensor or module 2700′ may have a height 2793′. For example, the height of the light barrier construct 2720′ at a central region of the sensor or module 2700′ may correspond to height 2793′. The height 2793′ may be a maximum distance the outer surface 2791′ extends perpendicularly away from the substrate 2716′ (e.g., toward the skin of a wearer). An outer region (e.g., along a perimeter of the substrate 2716′) of the sensor or module 2700′ may have a height 2795′. For example, the height of the light barrier construct 2720′ and/or opaque frame 2726′ at an outer region of the sensor or module 2700′ may correspond to height 2795′. The height 2795′ may be a minimum distance the outer surface 2791′ extends perpendicularly away from the substrate 2716′ (e.g., toward the skin of a wearer).

In some aspects, height 2793′ may be less than about 2.95 mm. In some aspects, height 2793′ may be less than about 2.90 mm. In some aspects, height 2793′ may be less than about 2.85 mm. In some aspects, height 2793′ may be less than about 2.80 mm. In some aspects, height 2793′ may be about 2.85 mm. In some aspects, height 2793′ may be less than about 2.70 mm. In some aspects, height 2793′ may be less than about 2.65 mm. In some aspects, height 2793′ may be less than about 2.60 mm. In some aspects, height 2793′ may be less than about 2.55 mm. In some aspects, height 2793′ may be about 2.58 mm.

In some aspects, height 2795′ may be less than about 1.40 mm. In some aspects, height 2795′ may be less than about 1.35 mm. In some aspects, height 2795′ may be less than about 1.30 mm. In some aspects, height 2795′ may be less than about 1.25 mm. In some aspects, height 2795′ may be about 1.29 mm. In some aspects, height 2795′ may be less than about 1.90 mm. In some aspects, height 2795′ may be less than about 1.85 mm. In some aspects, height 2795′ may be less than about 1.80 mm. In some aspects, height 2795′ may be less than about 1.75 mm. In some aspects, height 2795′ may be about 1.78 mm.

In some aspects, the height 2793′ may be greater (e.g., larger) than height 2795′. In some aspects, height 2793′ may be less than about 230% of height 2795′. In some aspects, height 2793′ may be less than about 225% of height 2795′. In some aspects, height 2793′ may be less than about 220% of height 2795′. In some aspects, height 2793′ may be less than about 215% of height 2795′. In some aspects, height 2793′ may be about 221% of height 2795′. In some aspects, height 2793′ may be less than about 155% of height 2795′. In some aspects, height 2793′ may be less than about 150% of height 2795′. In some aspects, height 2793′ may be less than about 145% of height 2795′. In some aspects, height 2793′ may be less than about 140% of height 2795′. In some aspects, height 2793′ may be about 145% of height 2795′.

Advantageously, a greater height 2793′ (and/or greater ratio of height 2793′ to 2795′) (for example, a taller light barrier at a central region of the sensor or module 2700 may cause light emitted from emitter chambers to travel a greater distance before reaching the detector chambers. Light that travels a greater distance may penetrate deeper into the tissue of the wearer which may improve accuracy of a physiological measurement. A smaller height 2793′ (and/or smaller ratio of height 2793′ to 2795′) may reduce discomfort to the wearer wearing the wearable device 10 or may reduce obstruction to blood flow of the wearer by reducing the amount of pressure the wearable device places on the wearer. The height 2793′ and/or height 2795′ may be selected to balance the above-mentioned considerations such as increasing the depth which light penetrates into the tissue and reducing discomfort or blood flow obstruction of the wearer.

FIG. 27O and FIG. 27P illustrate two example aspects of a sensor or module 2700′ with different light barrier construct configurations. FIGS. 27O and 27P also show an example light path from an emitter chamber to a detector chamber. The light barrier construct 2720′ (or portions thereof) shown in the example aspect of FIG. 27O may be taller (e.g., extending away from a surface of the substrate 2716) and/or wider than the light barrier construct 2720 (or portions thereof) shown in the example aspect of FIG. 27P. The greater height and/or width of the light barrier construct 2720′ in the aspect of FIG. 27O may cause the light emitted from an emitter chamber 2778′ to travel a greater distance before reaching a detector chamber and thus penetrate deeper into the tissue of the wearer than in the aspect of FIG. 27P. Thus, adjusting the height and/or width of the light barrier construct may affect the path the light travels from the emitter chamber to the detector chamber which may affect an accuracy of a physiological measurement. The height and/or width of the light barrier construct may be adjusted, according to various aspects, as required or desired.

FIG. 27Q illustrates a cutaway side view of an example sensor or module 2700′ showing light transmissive lens(es) or cover(s) 2702′ and light diffusing material. The light diffusing materials can be included in one or more of the emitter or detector chambers to improve distribution of emitted lighted and/or detected light. The diffusing materials or encapsulant, can include, for example, microspheres or glass microspheres. The encapsulant can eliminate air gaps between the surface of the light transmissive cover 2702′ and the emitters and/or the detectors. The encapsulant can be included around the emitters to more evenly spread the emitted light, causing the emitted light to appear to be emitted from an entire emitter chamber rather than from a point source (that is, a single LED emitter) if the encapsulant were absent. The light transmissive lens(es) or cover(s) 2702′ may include polycarbonate.

### Example Graphical User Interfaces of External Devices

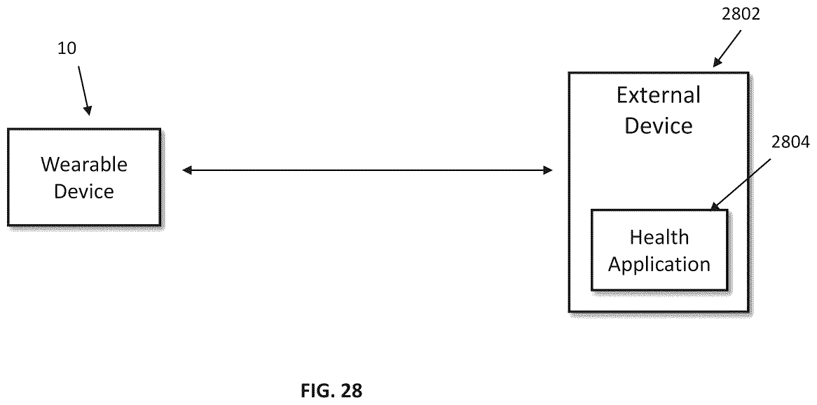

As discussed herein and as shown in FIG. 2, the wearable device 10 can be in communication, for example wirelessly, to an external device. FIG. 28 shows a block diagram illustrating an example aspect of the wearable device 10 in communication with an external device 2802. The communication may be wireless, such as, but not limited to, Bluetooth and/or near-field communication (NFC) wireless communication. As shown in FIG. 2, the wearable device 10 may be in communication with any number and/or types of external devices 2802 which may include a patient monitor 202 mobile communication device 204 (for example, a smartphone), a computer 206 (which can be a laptop or a desktop), a tablet 208, a nurses' station system 201, glasses such as smart glasses configured to display images on a surface of the glasses and/or the like. The external device 2802 may include a health application 2804. “External device” and “computing device” may be used interchangeably herein.

A user may operate the external device 2802 as described herein. A wearer may wear the wearable device 10. In some implementations, the user of the external device 2802 and the wearer of the wearable device 10 are different people. In some implementations, the user of the external device 2802 and the wearer of the wearable device 10 are the same person. The terms “user” and “wearer” and “patient” may be used interchangeably herein and may all refer to a person wearing the wearable device 10 and/or a person using the health application 2804 and their uses in any of the given examples are not meant to be limiting of the present disclosure.

The wearable device 10 may communicate information such as physiological data of the wearer/user to the external device 2802. The external device 2802 may display the physiological parameters received from the wearable device 10, as described herein.

The external device 2802 may control operation of the wearable device 10, for example via a wireless connection as described herein. For example, the external device 2802 may cause the wearable device 10 to start or stop taking measurements of a wearer's physiological parameters. In some aspects, the wearable device 10 may continuously measure and communicate a wearer's physiological parameters to the external device 2802. In some aspects, the external device 2802 may continuously display the wearer's physiological parameters received from the wearable device 10. In some aspects, the wearable device 10 may measure and communicate physiological parameters to an external device 2802 for a finite amount of time, such as 1 minute, upon receiving user input at the external device 2802 communicated to the wearable device 10.

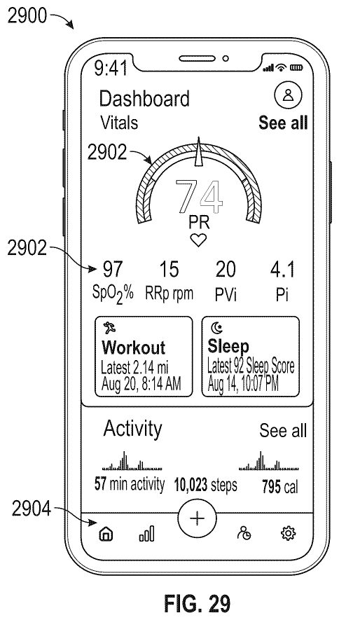

FIGS. 29, 30A-30H, 31A-31D, 32, 33A-33C, 34A-34C and 35A-35B illustrate example graphical user interfaces of a health application 2804, according to some aspects of the present disclosure. In various aspects, aspects of the user interfaces may be rearranged from what is shown and described below, and/or particular aspects may or may not be included. The health application 2804 can execute on the external device 2802 to present the graphical user interfaces of FIGS. 29, 30A-30H, 31A-31D, 32, 33A-33C, 34A-34C and 35A-35B. As described herein, the health application 2804 can receive a respective client configuration package that causes the presentation of the graphical user interfaces of FIGS. 29, 30A-30H, 31A-31D, 32, 33A-33C, 34A-34C and 35A-35B. The graphical user interfaces of FIGS. 29, 30A-30H, 31A-31D, 32, 33A-33C, 34A-34C and 35A-35B may have similar user interface elements and/or capabilities.

FIG. 29 illustrates an example dashboard user interface 2900 of the health application 2804. The dashboard user interface 2900 can display current physiological parameters 2902 of a wearer such as pulse rate, SpO₂, RRp, PVi, Pi and the like. In addition to the presentation of current wearer physiological parameter(s) 2902, the dashboard user interface 2900 can present indicator(s) associated with one or more of the physiological parameters 2902 that visually indicate a status of the parameters 2902 and various status ranges for each parameter 2902. The indicator(s) may be color coded or otherwise show a severity or status of a physiological parameter 2902. The dashboard user interface 2900 may additionally display a history of wearer statistics/information such as workout history information, sleep information, activity levels, steps taken, and/or calories burned.

The dashboard user interface 2900 may additionally display one or more navigation selectors 2904 configured for selection by a user. The one or more navigation selectors 2904 may include a home navigation selector, an activity navigation selector, a workout navigation selector, a vitals navigation selector, a sleep navigation selector, a history navigation selector, a share navigation selector and/or a settings navigation selector. Selection of the navigation selectors 2904 may cause the health application 2804 to display any of the graphical user interfaces described herein associated with the selected navigation selector 2904. The navigation selectors 2904 may be displayed in any of the graphical user interfaces described herein.

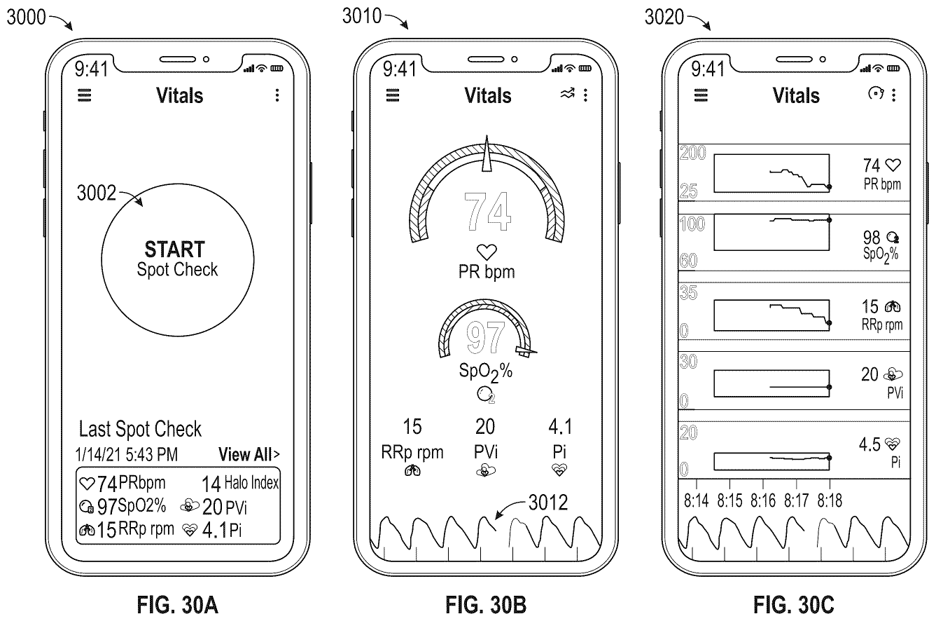

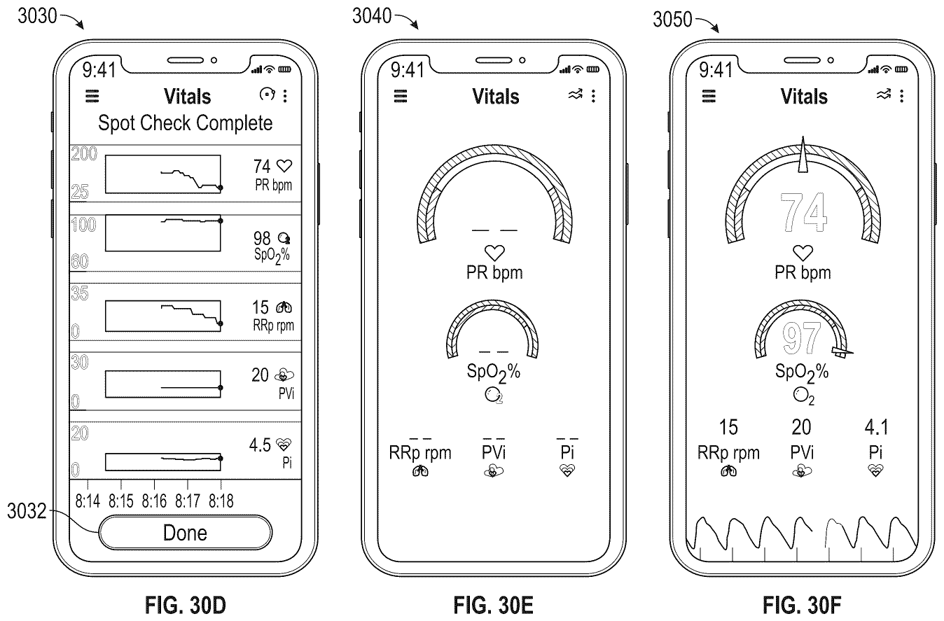

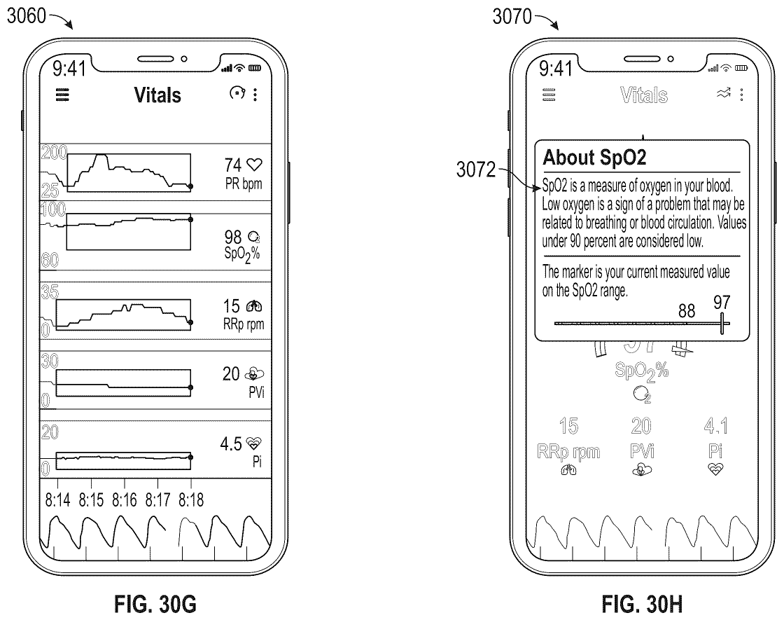

FIGS. 30A-30D illustrate example spot check monitoring user interfaces of the health application 2804. The wearable device 10 may be configured to perform spot checks. A spot check may be a discrete period of time during which the wearable device 10 performs physiological measurements of a wearer. For example, during a spot check the wearable device 10 may perform physiological measurements for one minute. A user/wearer may initiate a spot check at any desirable time, for example via the health application 2804 of the external device 2802.

FIG. 30A illustrates an example spot check monitoring user interface 3000 of the health application 2804. A user/wearer may initiate a spot check by selecting a start spot check selectable component 3002. Upon selection of the start spot check selectable component 3002 via the health application 2804, the external device 2802 may cause the wearable device 10 to commence measuring physiological parameters as described herein. In some aspects, upon selection of the start spot check selectable component 3002 via the health application 2804, the external device 2802 may check the power level of the wearable device 10 (for example, by checking a charge and/or voltage level of the power source 16) prior to causing the wearable device to start the spot check to determine if the wearable device 10 has sufficient power to perform the spot check. Upon determining that the wearable device 10 has sufficient power, the external application may cause the wearable device 10 to perform the spot check. Upon determining that the wearable device 10 does not have sufficient power, the external application may not cause the wearable device 10 to perform the spot check and/or may cause the spot check to be performed only for certain physiological parameters. In some aspects, upon receiving a command from the external device 2802 to perform a spot check, the wearable device 10 may check its power level (for example, by checking a charge and/or voltage level of the power source 16) prior to commencing physiological measurements for the spot check to determine if it has sufficient power to perform the spot check. Upon determining that it has sufficient power, the wearable device 10 may commence performing the spot check. Upon determining that it does not have sufficient power, the wearable device 10 may not perform the spot check.

In some aspects, the wearable device 10 may perform a spot check for a predetermined amount of time, for example three minutes, two minutes, one minute, 30 seconds, 15 seconds or any amount of time as required or desired. In some aspects, a user may select the predetermined amount of time for which the spot check is to be performed, for example before or after selecting the selectable component 3002. In some aspects, the wearable device 10 may perform a spot check until it receives a command to stop performing the spot check. For example, the external device 2802 may cause the wearable device 10 to stop performing a spot check upon receiving a user command, for example via a selectable component (e.g., similar to selectable component 3002) of the health application 2804.

FIG. 30B illustrates an example spot check monitoring user interface 3010 of the health application 2804. The spot check monitoring user interface 3010 may display the substantially real-time measurements of physiological parameters as measured by the wearable device 10 during the spot check such as pulse rate, SpO₂, RRp, PVi, Pi and the like. As described herein, substantially real-time measurements, waveforms, parameters and/or the like may refer to physiological measurements that are displayed (e.g., via user interfaces of the health application) at the same time or substantially the same time (e.g., neglecting any small delays such as those that are imperceptible to humans such as delays arising from electrical conduction or transmission) that they are obtained by the wearable device 10. The spot check monitoring user interface 3010 may display the substantially real-time waveforms 3012 of any of the physiological parameters such as pulse waveform and/or plethysmograph waveform obtained during the spot check.

FIG. 30C illustrates an example spot check monitoring user interface 3020 of the health application 2804. The spot check monitoring user interface 3020 may display substantially real-time measurements of wearer physiological parameters such as those described with reference to FIG. 30B. The spot check monitoring user interface 3020 can further include historical data. In particular, the spot check monitoring user interface 3020 can include a visualization(s) that present historical trends of wearer physiological parameters. As shown, the visualization(s) can include one or more graphs with an x-axis of time and a y-axis of parameter values.

FIG. 30D illustrates an example spot check monitoring user interface 3030 of the health application 2804. The spot check monitoring user interface 3030 may be displayed when the wearable device 10 has completed the spot check. In some aspects, as depicted in FIG. 30D, the user interface 3030 may display physiological parameters obtained during the spot check in a similar format to that shown in user interface 3020 of FIG. 30C. In some aspects, the user interface 3030 may display physiological parameters obtained during the spot check in a similar format to that shown in user interface 3010 of FIG. 30B.

In some aspects, the spot check monitoring user interface 3030 may be displayed for a predetermined length of time, such as one minute, after the spot check has terminated, and after the predetermined length of time the health application 2802 may display another user interface such as any of the user interfaces described herein, such as the dashboard user interface 2900 or the spot check monitoring user interface 3000. In some aspects, a user may navigate to another user interface such as any of the user interfaces described herein, such as the dashboard user interface 2900 or the spot check monitoring user interface 3000, upon selection of the selectable component 3032. In some aspects, the health application 2804 may display the user interface 3030 until a user selects the selectable component 3032.

FIGS. 30E-30H illustrate example continuous monitoring user interfaces of the health application 2804. The wearable device 10 may be configured to perform continuous measurements of physiological parameters of a wearer. For example, the wearable device may continuously measure a wearer's physiological parameters indefinitely, for example as long as the device is powered on or until a user selects to cease the continuous operation mode and/or until a user selects operation of a non-continuous monitoring mode of operation such as the spot check as described herein.

In some aspects, the external device 2802 may check the power level of the wearable device 10 (for example by checking a charge and/or voltage level of the power source 16) prior to causing the wearable device to enter a continuous monitoring mode and/or during continuous monitoring to determine if the wearable device 10 has sufficient power to perform continuous monitoring. Upon determining that the wearable device 10 has sufficient power, the external application may cause the wearable device 10 to initiate continuous monitoring and/or to continue continuous monitoring. Upon determining that the wearable device 10 does not have sufficient power, the external application may not cause the wearable device 10 to initiate continuous monitoring and/or may cause the wearable device 10 to discontinue continuous monitoring, and/or may cause the wearable device 10 to initiate a mode of operation that is different than continuous monitoring such as a modified continuous monitoring mode or a spot check mode. In some aspects, upon receiving a command, for example from the external device 2802 to perform continuous monitoring, the wearable device 10 may check its power level (for example by checking a charge and/or voltage level of the power source 16) prior to commencing and/or during continuous monitoring to determine if it has sufficient power to perform continuous monitoring. Upon determining that it has sufficient power, the wearable device 10 may commence performing continuous monitoring and/or may continue performing continuous monitoring. Upon determining that it does not have sufficient power, the wearable device 10 may not initiate continuous monitoring and/or may discontinue continuous monitoring, and/or may cause the wearable device 10 to initiate a mode of operation that is different than continuous monitoring such as a modified continuous monitoring mode or a spot check mode.

FIG. 30E illustrates an example continuous monitoring user interface 3040 of the health application 2804. User interface 3040 may be displayed when the wearable device 10 is operating in continuous monitoring mode and is simultaneously unable to obtain physiological measurements (or the physiological measurements obtained are determined to fall below an accuracy threshold), for example when the wearable device 10 not being worn (or not being worn properly) by a wearer.

FIG. 30F illustrates an example continuous monitoring user interface 3050 of the health application 2804. The continuous monitoring user interface 3050 may display the substantially real-time measurements of physiological parameters as measured by the wearable device 10 during continuous monitoring in a similar manner as explained with reference to the display of physiological parameters during a spot check in example user interface 3010 of FIG. 30B.

FIG. 30G illustrates an example continuous monitoring user interface 3060 of the health application 2804. The continuous monitoring user interface 3060 may display substantially real-time measurements of wearer physiological parameters such as those described with reference to FIG. 30F or FIG. 30B. The continuous monitoring user interface 3060 can further include historical data. In particular, the continuous monitoring user interface 3060 can include a visualization(s) that present historical trends of wearer physiological parameters. As shown, the visualization(s) can include one or more graphs with an x-axis of time and a y-axis of parameter values.

FIG. 30H illustrates an example continuous monitoring user interface 3070 of the health application 2804. The continuous monitoring user interface 3070 may display information 3702 relating to any of the physiological parameters discussed herein such as those displayed in any of the graphical user interfaces discussed herein. The information 3072 may help a user/wearer understand the physiological parameter. In the example of FIG. 30H, the continuous monitoring user interface 3070 displays information 3072 relating to SpO₂ physiological parameter. The continuous monitoring user interface 3070 may display information relating to a physiological parameter in response to a user selection of the displayed corresponding physiological parameter. In some aspects, as depicted in FIG. 30H, a user may select a physiological parameter from a displayed format similar to that shown in user interface 3050 of FIG. 30F to display information 3072. In some aspects, a user may select a physiological parameter from a displayed format similar to that shown in user interface 3060 of FIG. 30G to display information 3072.

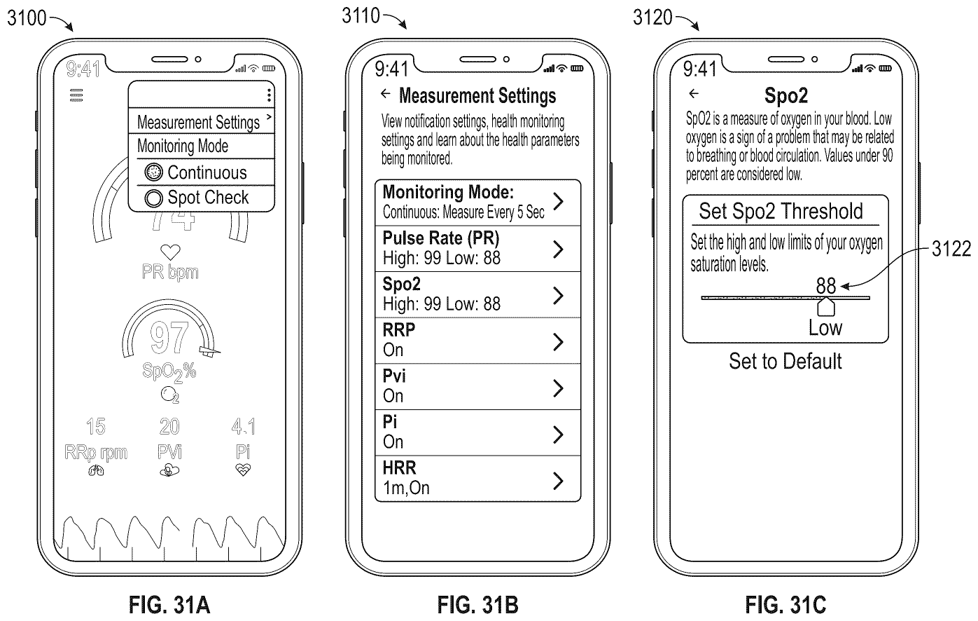

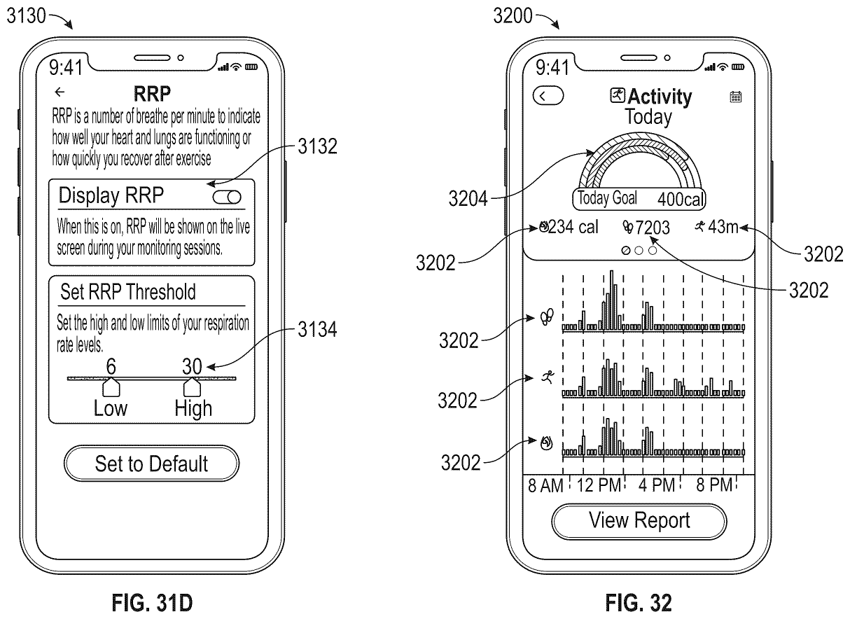

FIGS. 31A-31D illustrate example measurement settings user interfaces of the health application 2804. As discussed with reference to the example measurement settings interfaces, a user may adjust the settings of the health application 2804 with regards to at least the physiological measurements and/or may adjust the mode of operation of the wearable device 10.

FIG. 31A illustrates an example measurement settings user interface 3100 of the health application 2804. The measurement settings user interface 3100 may allow a user to select a mode of operation of the wearable device 10. For example, a user may select, via user interface 3100, whether the wearable device operates in continuous monitoring mode or spot check mode. A user may also navigate, via selection in the user interface 3100, to additional measurement settings user interfaces such as user interface 3110.

FIG. 31B illustrates an example measurement settings user interface 3110 of the health application 2804. User interface 3110 may display various physiological parameters, as shown. A user may select any of the displayed physiological parameters to edit and/or adjust the settings associated with the selected physiological parameter. User selection of the physiological parameters may navigate a user to additional measurement settings user interfaces such as user interfaces 3120 or 3130.

FIG. 31C illustrates an example measurement settings user interface 3120 of the health application 2804. User interface 3120 may allow a user to set one or more threshold levels relating to a physiological parameter such as any of the physiological parameters measured by the wearable device 10 and/or displayed by the graphical user interfaces as discussed herein. In some aspects, when the physiological parameter value as measured by the wearable device 10 reaches the threshold level, the external device 2802 may generate a notification. For example, if a threshold level is breached, then the external device 2802 can cause an emergency notification to be generated. The emergency notification may be displayed on the graphical user interface of the external device 2802 and/or communicated to another device such as the wearable device 10, another external device 2802 and/or a third-party device. In some aspects, the generation of physiological parameter notification(s) can be activated or deactivated by the user.

The example measurement settings user interface 3120 displays a slidable component 3122 relating to SpO₂ threshold. A user may slide the slidable component 3122 to adjust and/or select the threshold SpO₂ limit. As shown, a user has set the SpO₂ threshold limit to 88%. As discussed herein, in some aspects, when the wearable device 10 measures an SpO₂ level of the wearer to be 88% or less than 88%, the health application 2804 of the external device 2802 may generate a notification and/or alert.

FIG. 31D illustrates an example measurement settings user interface 3130 of the health application 2804. The example measurement settings user interface 3130 displays a slidable component 3134 relating to RRp threshold. A user may slide the slidable component 3134 to adjust and/or select an upper and lower RRp threshold limit. As shown, a user has set the RRp lower threshold limit to 6 and has set the RRp upper threshold limit to 30. As discussed herein, in some aspects, when the wearable device 10 measures an RRp level of the wearer to be less than 6 or greater than 30, the health application 2804 of the external device 2802 may generate a notification and/or alert.

The example measurement settings user interface 3130 displays a toggle component 3132 relating to RRp display. A user may select the toggle component 3132 to enable or disable the display of RRp physiological parameter measurements in any of the user interfaces discussed herein, for example user interfaces 29 or 30A-30H. A toggle component or other means similar to the toggle component 3132 shown in user interface 3130 may be displayed in other user interfaces relating to other physiological parameters. For example, a similar toggle component may be displayed in user interface 3120 to allow a user to enable or disable the display of SpO₂ physiological parameter measurement in the user interfaces discussed herein.

FIG. 32 illustrates an example activity user interface 3200 of the health application 2804. The activity user interface 3200 may display a history of activity level of the wearer for any number of activity categories 3202 such as the number of steps taken, distance travelled, and/or calories burned. The activity user interface 3200 may display the activity levels of the wearer throughout a time period such as a 12 hour time period, a 24 hour time period, a week time period or any other time period. For example, the activity user interface 3200 can include a visualization(s) that presents historical trends of wearer activity levels 3202. As shown, the visualization(s) can include one or more graphs with an x-axis of time and a y-axis of activity level. The activity user interface 3200 may display goals of the wearer/user for any of the activity categories 3202 and may display a visualization 3204 corresponding to the goal and/or a wearer's status in achieving the goal.

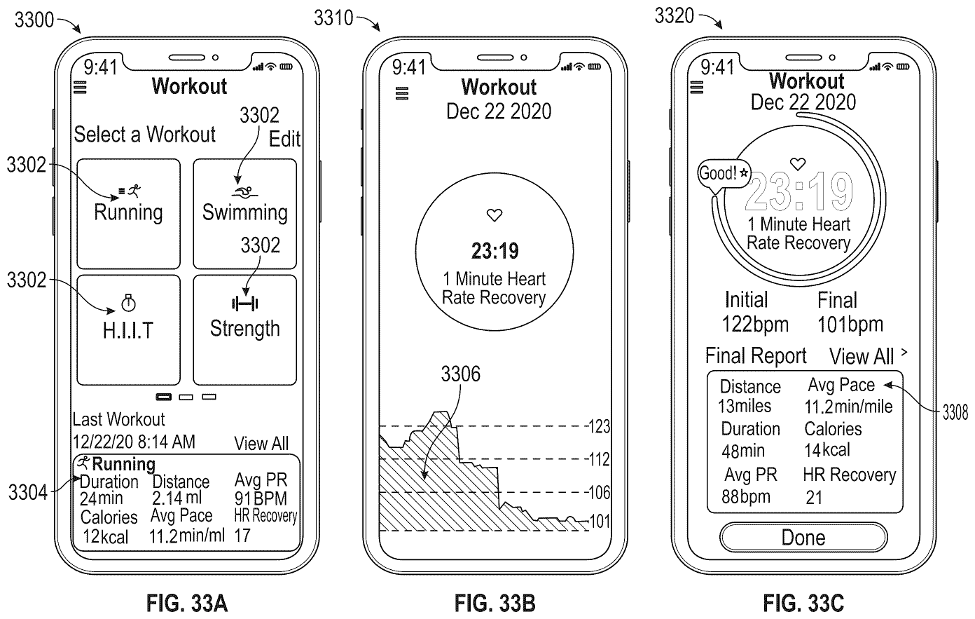

FIG. 33A illustrates an example workout user interface 3300 of the health application 2804. The user may select a selectable component 3302 of the workout user interface 3300 to cause the wearable device to begin monitoring wearer physiological parameters and/or to cause the health application 2804 to begin tracking/logging wearer physiological parameters during the workout. The selectable component 3302 may correspond to the type of workout such as running, swimming, high-intensity interval training (H.I.I.T), strength training and the like. The workout user interface 3300 may display information 3304 relating to a previous workout.

FIG. 33B illustrates an example workout user interface 3310 of the health application 2804. The workout user interface 3310 may be displayed by the health application 2804 during and/or after a workout of the wearer. The workout user interface 3310 may display physiological parameters of the wearer, such as received by the wearable device 10, and other information relating to the workout such as time duration of the workout. The workout user interface 3310 may display historical and/or substantially real-time physiological parameters of the wearer. The workout user interface 3310 may display a visualization(s) 3306 of any of the wearer physiological parameters. For example, the workout user interface 3310 can include a visualization(s) that present historical trends of wearer physiological parameters during the workout. As shown, the visualization(s) can include one or more graphs with an x-axis of time and a y-axis of parameter values.

FIG. 33C illustrates an example workout user interface 3320 of the health application 2804. The workout user interface 3320 may be displayed by the health application 2804 after a workout of the wearer. The workout user interface 3320 may display information 3308 relating to the workout of the wearer.

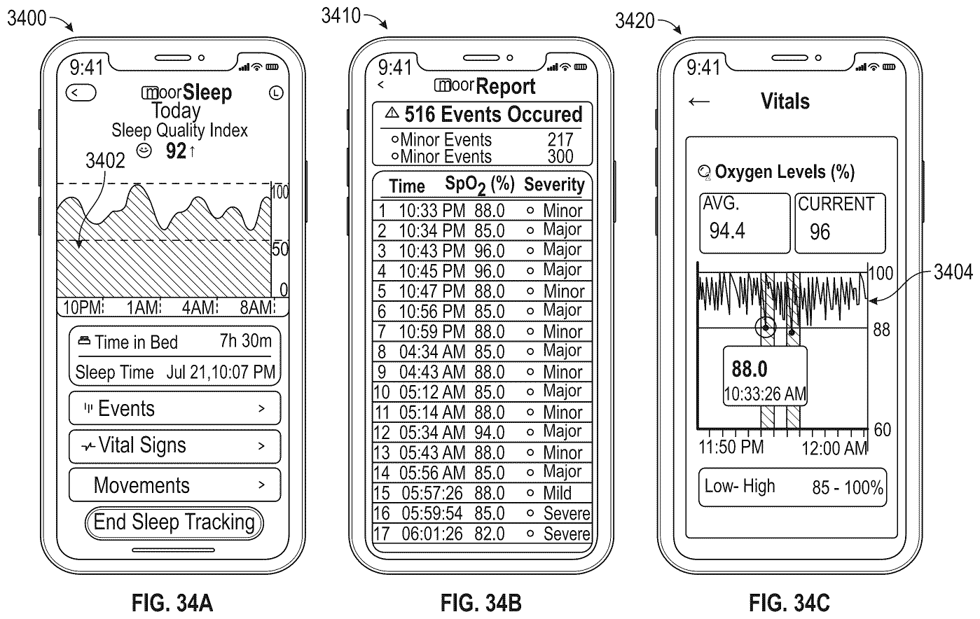

FIG. 34A illustrates an example sleep user interface 3400 of the health application 2804. The sleep user interface 3400 may display a sleep quality index which may be calculated based, at least in part, on one or more of the physiological parameters of the wearer received from the wearable device 10. The sleep user interface 3400 may display a visualization(s) 3402 of the sleep quality index. For example, the sleep user interface 3400 can include a visualization(s) that present historical trends of the sleep quality index during a time the wearer is asleep. As shown, the visualization(s) can include one or more graphs with an x-axis of time and a y-axis of sleep quality index.

FIG. 34B illustrates an example sleep user interface 3410 of the health application 2804. The sleep user interface 3410 may display a history of events occurring during a time the wearer is asleep. The events may be major or minor events as determined by, for example, a magnitude of the event. For example, the history of events may display all the times during which the wearer was asleep wherein a physiological parameter, such as SpO₂, exceeded a threshold. The threshold(s) may be set by the user/wearer as discussed with reference to FIGS. 31C-31D.

FIG. 34C illustrates an example sleep user interface 3420 of the health application 2804. The sleep user interface 3420 may display physiological parameters of the wearer, such as current physiological parameters and/or average physiological parameters of the wearer during a time the wearer was asleep. The sleep user interface 3420 may display a visualization(s) 3404 of the physiological parameters. For example, the sleep user interface 3420 can include a visualization(s) that present historical trends of the physiological parameters during a time the wearer is asleep. As shown, the visualization(s) can include one or more graphs with an x-axis of time and a y-axis of parameter value. The visualization may further include an indication of a threshold(s) and may show all times wherein the physiological parameter exceeded the threshold(s). The threshold(s) may correspond to the threshold(s) discussed with reference to FIG. 34B and may be set by the wearer as discussed with reference to FIGS. 31C-31D.

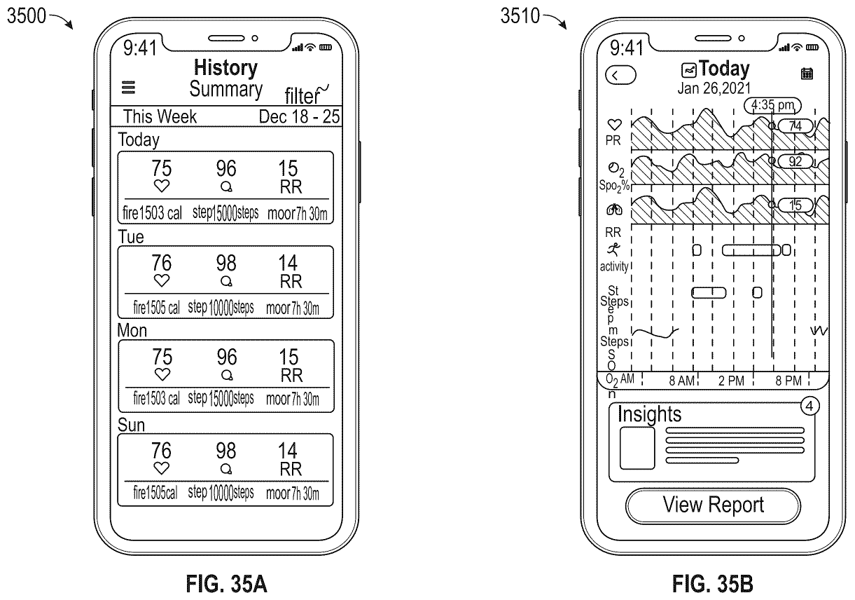

FIG. 35A illustrates an example history user interface 3500 of the health application 2804. The history user interface 3500 may display historical data, for example historical data of the wearer physiological parameters received from the wearable device 10. The history user interface 3500 may display historical data over any range of time, for example historical data for the day and/or historical data for the week. The history user interface 3500 may display historical data grouped according to any time frame, for example historical data grouped by day and/or historical data grouped by hour. As shown in FIG. 35A, the history user interface 3500 may display historical data for the current day and any number of preceding days.

FIG. 35B illustrates an example history user interface 3510 of the health application 2804. The history user interface 3510 may display historical data, such as historical physiological parameter values and/or activity levels of the wearer. The history user interface 3510 can include a visualization(s) that present historical trends of wearer physiological parameters and/or activities, such as calories burned or steps taken. As shown, the visualization(s) can include one or more graphs with an x-axis of time and a y-axis of values, such as physiological parameter values or activity levels. The history user interface 3510 may display the visualization(s) such that the x-axis of time of one graph corresponds with the x-axis of time of any of the other graphs. The history user interface 3510 may further display historical data value(s) of the visualization graph(s) corresponding to the time of the of the visualization graph(s) upon selection by a user such as by adjusting a slider along the x-axis of the graphs as shown in FIG. 35B.

Appendix A that is attached to this specification is hereby incorporated by reference in its entirety and is made a part of this specification. Appendix A shows additional example graphical user interfaces of the health application 2804. A1 illustrates example user interfaces of the wearable device 10 for connecting the wearable device 10 with an external device 2802. A2 illustrates an example user interface for initiating the health application 2804 on an external device 2802. A3-A5 illustrate example user interfaces for creating an account on the health application 2804. A6 illustrates an example user interface of a successful account creation for health application 2804. A7 illustrates example user interfaces of successfully pairing the wearable device 10 with an external device 2802. A8 illustrates example user interfaces for setting goals in the health application 2804. A9 illustrates example user interfaces of unsuccessfully logging in to the health application 2804.

A10 illustrates example user interfaces of a homepage of the health application 2804. A11-A13 illustrate example user interfaces of a dashboard of the health application 2804. A14 illustrates example user interfaces for spot check monitoring of wearer physiological parameters with the wearable device 10. As shown in A14, a user may select a component via the user interface to start a physiological measurement with the wearable device 10. The wearable device 10 may measure physiological parameters and display the measurements to the user interface for a predetermined amount of time (e.g., one minute) and/or until the user selects a component via the user interface to stop the physiological measurement. A15 illustrates an example user interface for displaying physiological parameters measured continuously with the wearable device 10. A16 illustrates example user interfaces for adjusting physiological measurement settings. A17 illustrates an example user interface for displaying physiological parameter measurements.

A18-A19 illustrates example user interfaces for displaying activity of a user. A20-A21 illustrate example user interfaces for displaying workout information of a user. A22-A24 illustrate example user interfaces for displaying sleep information of a user. A25-A26 illustrates example user interfaces for setting sleep settings for a user. A27-A29 illustrate example user interfaces for displaying a history of a user's physiological parameter measurements and activity.

A30-A36 illustrate example user interfaces for setting the settings of the wearable device 10.

A37 illustrates example user interfaces of a dashboard of the health application 2804. A38 illustrates example user interfaces of a history of physiological parameters of a user. A39 illustrates an example user interface for sharing information, such as physiological parameters, from the external device 2802 to another device. A40-A41 illustrate example user interfaces for navigating between interfaces of the health application 2804 using an interactive navigation bar displayed at the bottom of the user interface. A42 illustrates example user interfaces of a dashboard and account homepage of the health application 2804. A43 illustrates example user interfaces for navigating between interfaces of the health application 2804 using an interactive navigation bar displayed at the bottom of the user interface. A44 illustrates example user interfaces of a dashboard of the health application 2804.

### Example Graphical User Interfaces of the Wearable Device

As discussed herein, for example, with reference to FIG. 11A, the wearable device 10 can include a display 12, which can include the display screen and touch input from the wearer. For example, the display 12 may comprise a capacitive touchscreen configured to receive touch input from the wearer to control functionality of the wearable device 10. The wearable device 10 can also include user interfaces on the device 10 to receive input from the wearer. As shown in FIG. 1D, example user interfaces can include buttons 13.

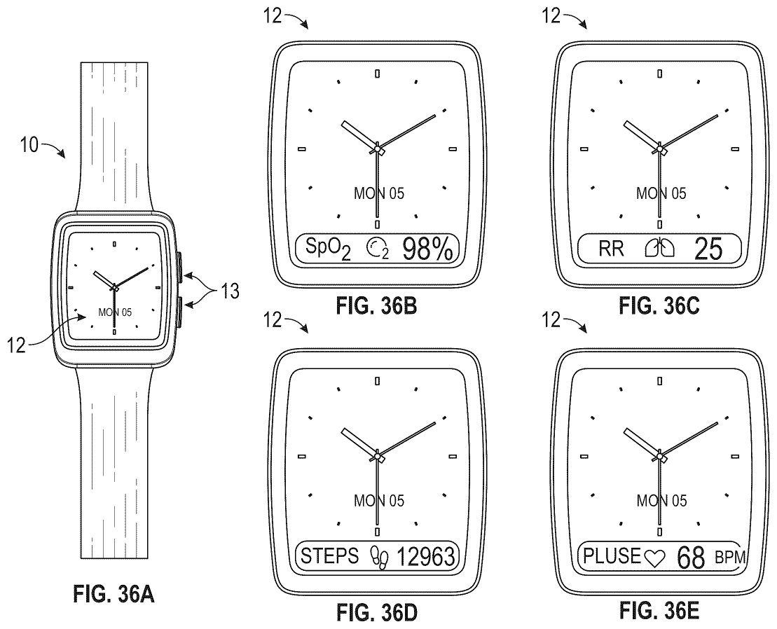

FIG. 36A shows an example wearable device 10 including a display 12 and buttons 13. FIGS. 36B-36E show example screens of the display 12 of the wearable device 10. The display 12 may be configured to display many different screens. For example, in some aspects, the display 12 may display a screen with various physiological parameter information (such as values and trends) and in other aspects, the display 12 may display a screen with no physiological parameter information. In some aspects, the display 12 may display a screen with non-physiological related information such as date, time and other notifications.

The display 12 may display various screens in a cyclical manner. For example, the display 12 may display a screen with a first physiological parameter information (such as value and trend) and then may display a screen with a second physiological parameter information and then may display a screen with a third physiological parameter information and so forth. In some aspects, the order of the screens the display 12 displays is constant. The display 12 may cycle through displaying various screens automatically and/or manually. For example, the display 12 may change the screen that is displayed automatically, without user input, every 1 minute, every 30 seconds, every 10 seconds, every 5 seconds, every 3 seconds, every 2 seconds, every 1 second or any other time frame that is required or desired. In some aspects, a wearer/user may select the time frame (e.g., frequency) in which the display 12 will change the screen. In some aspects, a wearer/user may manually change the screen that is displayed by the display 12. For example, a wearer may touch the display 12 to provide touch input to the wearable device 10 and/or may press a button 13 to provide input to the wearable device 10. Upon receiving said input from the wearer, the display 12 may change the screen that is displayed. The display 12 may not change the screen again until another user input, as described, is received.

In some aspects, the display 12 may cycle through displaying various screens automatically and manually, as simultaneous modes of operation. For example, the display 12 may cycle through the various screens automatically without user input (e.g., every 3 seconds, for example) unless or until a wearer/user provides input (via, the display 12 and/or buttons 13) upon which the display 12 will change the screen.

In some aspects, the display 12 may cycle through displaying the various screens automatically or manually, but not both modes of operation at the same time. For example, the display 12 may cycle through the various screens automatically (e.g., every 3 seconds, for example) regardless of user input that is received. As another example, the display 12 may cycle through the various screens manually, only upon receiving user input, regardless of time that has elapsed. In some aspects, the display 12 may toggle between cycling through the displaying the various screens automatically and manually, for example upon adjustment of user settings of the wearable device 10.

FIGS. 36A-36E show example screens of the display 12 of the wearable device 10. FIG. 36A shows a screen without physiological parameter information (such as values and trends). FIG. 36B shows a screen with SpO2 physiological parameter value of the wearer. FIG. 36C shows a screen with RR physiological parameter value of the wearer. FIG. 36D shows a screen with the number of steps taken by the wearer. FIG. 36E shows a screen with PR physiological parameter value of the wearer. The display 12 may cycle through displaying the various screens of FIGS. 36A-36E automatically and/or manually, as described above.

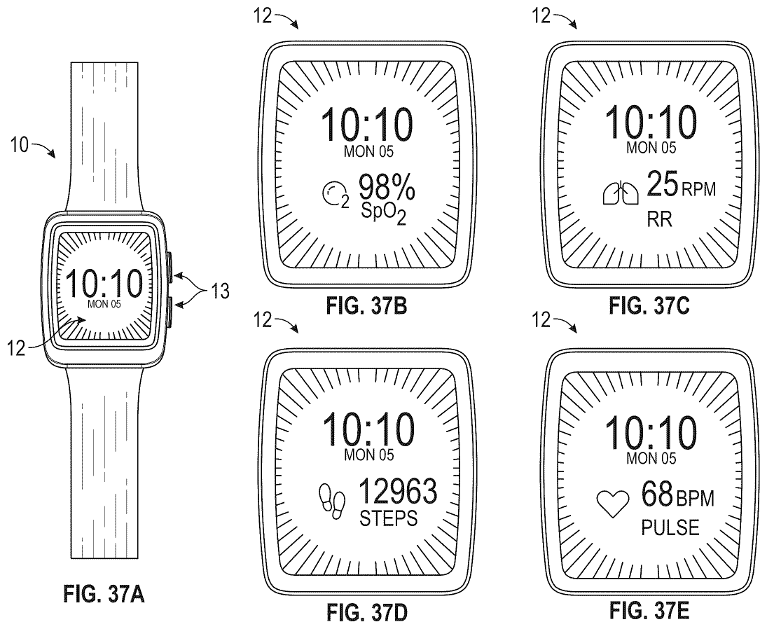

FIGS. 37A-37E show example screens of the display 12 of the wearable device 10. The display 12 may cycle through displaying the various screens of FIGS. 37A-37E automatically and/or manually, as described above.

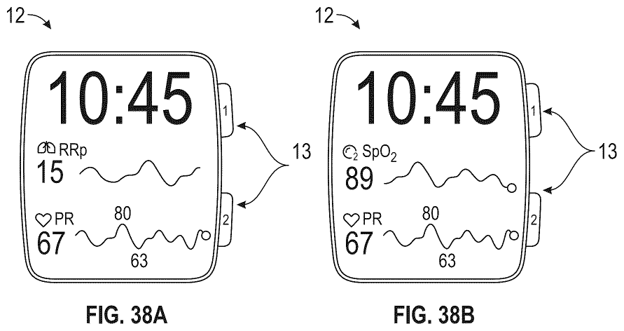

FIG. 38A shows an example screen of the display 12 of the wearable device 10. The display 12 displays a screen with time, and RRp physiological parameter value and trend and PR physiological parameter value and trend. In some aspects, the display 12 may cycle through displaying physiological information (such as values and trends) of other parameters (such as SpO2, steps taken etc.) automatically and/or manually as described above with reference to FIGS. 37A-37E, for example. In some aspects, a user/wearer may select physiological information to be displayed as a default by display 12, for example by adjusting the settings of the wearable device 10.

FIG. 38B shows an example screen of the display 12 of the wearable device 10. The display 12 displays a screen with time, and SpO2 physiological parameter value and trend and PR physiological parameter value and trend. In some aspects, the display 12 may automatically display physiological information (such as value and trend) relating to a parameter if that parameter has exceeded a threshold. For example, if a physiological parameter value exceeds a high or low threshold, the display 12 may automatically display that physiological parameter value and trend. This may be in place of physiological information of another parameter already displayed. For example, as shown in FIG. 38B, the wearer may have an SpO2 parameter value of 89. This value may be below a low limit threshold for SpO2 (for example, as set by a user/wearer as described with reference to FIG. 31C, for example). Upon determining that that the SpO2 has fallen below a low limit threshold, the wearable device 10 may cause the display 12 to display the physiological information (such as value and trend) for SpO2. This may replace physiological information of another parameter already displayed that has not exceeded a threshold. In this example, the display 12 replaces RRp (which has not exceeded any thresholds) in the screen (shown in FIG. 38A) with SpO2 in the screen (shown in FIG. 38B). Physiological parameters that have exceeded a threshold that are displayed in the screen by display 12 may differ in color or some other respect to physiological parameters that have not exceeded a threshold.

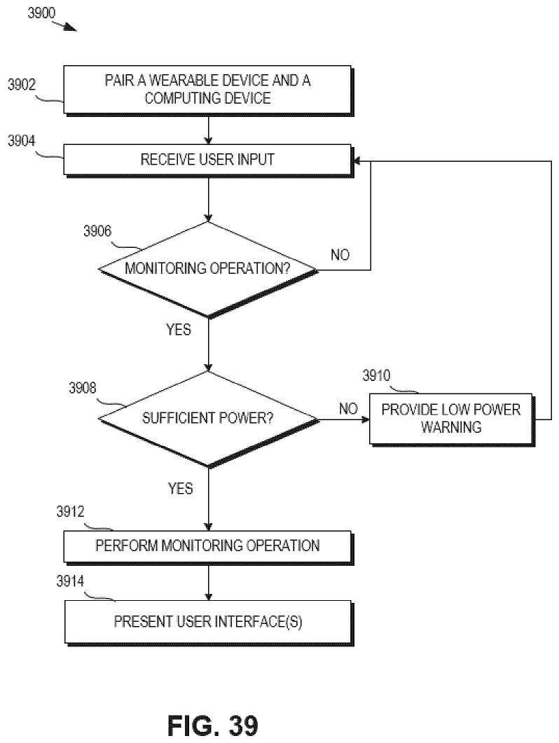

FIG. 39 is a flowchart illustrating an example process 3900 for performing monitoring operation(s) of one or more physiological parameters of a user/wearer. The process 3900 may be performed by a system that includes a wearable device 10 and a computing device 2802. The computing device 2802 may be, for example, any of the devices discussed with reference to FIG. 2, such as a patient monitor 202, a mobile communication device 204, a computer 206, a tablet 208, a nurses' station system 210 or any other device as required or desired. As discussed herein, the wearable device 10 and the computing device 2802 may be in communication with each other, for example, via Bluetooth technology or near-field communication (NFC) technology.

At block 3902, the wearable device 10 and the computing device 2802 may establish communication with each other. For example, the wearable device 10 may be paired with the computing device using Bluetooth or NFC.

At block 3904, the computing device 2802 may receive a user input. For example, a user can provide the user input via an interactive user interface of the computing device 2802. Example user input can include a user selection to initiate a monitoring operation, such as sport check or continuous monitoring. Additionally or alternatively, the wearable device 10 can receive user input, such as a user selection to initiate a monitoring operation, such as sport check or continuous monitoring.

At block 3906, the computing device 2802 may check to determine whether it has received user input corresponding to an instruction to perform a monitoring operation. In response to receiving the user input for a monitoring operation, the computing device 2802 may generate a signal to communicate to the wearable device 10, such as by sending a wireless communication to the wearable device 10. The signal may instruct the wearable device 10 to perform one or more operations according to the user input. For example, via a user interface of the computing device 2802, a user may select a spot check physiological monitoring operation. In response to the user input, the computing device 2802 may generate a signal to wirelessly communicate to the wearable device 10 to instruct the wearable device 10 to perform a spot check monitoring operation.

If the user input is not associated with a monitoring operation, the process 3900 can return to block 3904 to wait for additional input. In some aspects, the computing device 2802 can perform some other operation associated with the non-monitoring operation user input, as described herein.

The wearable device 10 may check to determine whether it has received a signal instructing it to perform a monitoring operation. If no signal has been received to perform a monitoring operation, the wearable device 10 may continue to determine whether it has received a signal at block 3906. If the wearable device 10 has received a signal, for example from the computing device 2802, instructing it to perform a monitoring operation, the process 3900 may continue to step 3908.

At block 3908, it may be determined whether the wearable device 10 has sufficient power to perform the requested monitoring operation. One or more techniques can be used to determine whether a battery has sufficient charge to initiate the particular monitoring operation. For example, the wearable device 10 may determine a current charge status of its battery. In some aspects, the wearable device 10 may include an estimate power consumption rate for respective operations and/or sensors. Additionally or alternatively, the wearable device 10 or another computing device 2802 may log power consumption for particular operations or sensors of wearable device(s) to determine an estimated power consumption rate. A determination of sufficient power for an operation can be based on the particular operation. For example, a spot check operation can use less power than a continuous monitoring operation. Moreover, an estimation of power consumption can be based on the particular operation. The estimation of power consumption can include an estimated duration of the operation. For example, a spot check operation can last a predetermined period of time, such as 1 second, 5 seconds, or 15 seconds, and the estimated power consumption can be based on that duration and the particular sensor(s) involved in the operation. Similarly, a continuous monitoring operation can have an estimated duration that is used for power consumption purposes, such as an estimated runtime for thirty minutes, one hour, four hours, or eight hours. The wearable device 10 or another computing device 2802 can estimate the power consumption for the operation by multiplying the estimated power consumption rate with the predetermined or estimated duration for the particular operation. The estimated power consumption can then be compared to the current battery charge level. For example, if the estimated power operation is greater than the current battery charge or is estimated to leave the battery at less than five percent or ten percent capacity, the wearable device 10 or another computing device 2802 can determine that there is insufficient power for the requested monitoring operation. If the wearable device 10 has sufficient power to perform the requested monitoring operation, the process proceeds to block 3912 to perform the monitoring operation.

If the wearable device 10 does not have sufficient power, the process 3900 proceeds to block 3910 to provide a lower power warning.

At block 3910, a low power warning can be provided. For example, the wearable device 10 may transmit a signal (such as a message) to the computing device 2802 that the wearable device 10 has insufficient power for the monitoring operation. The signal may cause the computing device 2802 to display a lower power warning message to a user.

In some aspects, if the wearable device 10 does not have sufficient power to perform the requested monitoring operation, the wearable device may continue to step 3912 to perform a monitoring operation that is different than the requested monitoring operation. For example, if the wearable device 10 does not have sufficient power to perform the requested operation, the wearable device 10 may determine a modified monitoring operation that may be performed, such as a modified continuous monitoring operation and/or a modified spot check monitoring operation. The determination may be based at least in part on the power of the monitoring device (e.g., remaining battery life). For example, a user may request a continuous monitoring operation and upon determining that there is insufficient power to perform the continuous monitoring operation (e.g., at step 3908) the wearable device 10 may instead perform a spot check operation (which may require less power than continuous monitoring) for an amount of time that is commensurate with the amount of power (e.g., remaining battery life) of the wearable device 10. As another example, a user may request a spot check monitoring operation to be performed for an amount of time and upon determining that there is insufficient power to perform the spot check monitoring operation (e.g., at step 3908) for the duration of the length of time the wearable device 10 may instead perform a spot check operation for an abbreviated length of time (which may require less power than the request spot check monitoring) which may be commensurate with the amount of power (e.g., remaining battery life) of the wearable device 10.

At block 3912, a monitoring operation may be performed. For example, the wearable device 10 may perform the monitoring operation. As described herein, example monitoring operations can include one or more of a spot-check monitoring operation, a continuous monitoring operation, and/or any other monitoring operation as discussed herein. The wearable device 10 may perform the monitoring operation on the wearer/user for one or more of the physiological parameters, such as, but not limited to, SpO2, RR, PR, RRp, etc.

The wearable device 10 may transmit physiological parameter measurement data to the computing device 2802. In some aspects, the wearable device 10 may transmit measurement data during the monitoring operation (such as while the wearable device 10 measures the physiological parameters and/or during a continuous monitoring operation).

At block 3914, one or more user interfaces may be presented. For example, the computing device 2802 may present the physiological parameter measurement data received from the wearable device 10 in a user interface. Similarly, the wearable device 10 can present the physiological parameter measurement data in a user interface on the wearable device 10. In response to receiving physiological parameter measurement data from the wearable device 10, the computing device 2802 may display the user interfaces described herein, such as with reference to FIGS. 30B, 30C, 30F and/or 30G. The computing device 2802 may display the physiological parameter measurement data in substantially real-time, for example, as the wearable device 10 performs the monitoring operation and measures the one or more physiological parameters.

In some implementations, the wearable device 10 may generate user interface data to display physiological parameter measurement data to a wearer/user of the wearable device 10. For example, the wearable device 10 may display the user interfaces described herein on a display screen 12 of the wearable device 10, such as with reference to FIGS. 38A-38B. The wearable device 10 may display the physiological parameter measurement data in substantially real-time, for example, as the wearable device 10 performs the monitoring operation and measures the one or more physiological parameters.

### Additional Implementation Details

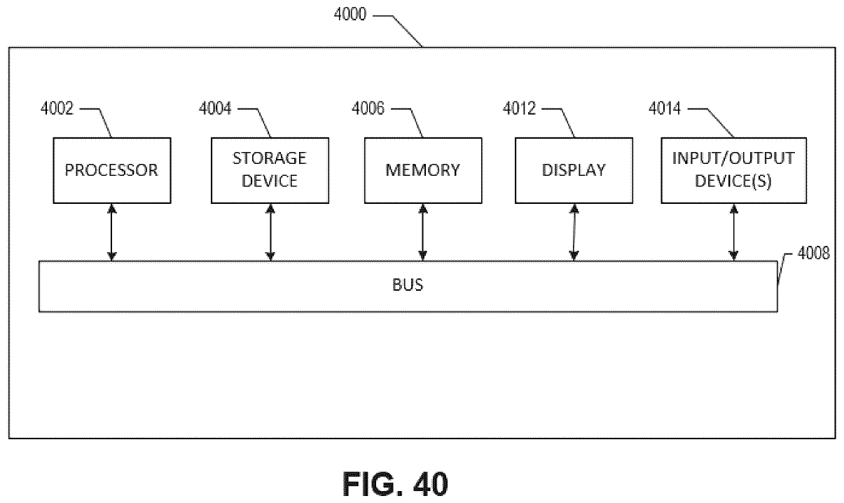

FIG. 40 is a block diagram that illustrates example components of a computing device 4000. The computing device 4000 can implement aspects of the present disclosure, and, in particular, aspects of the wearable device 10 and/or the computing device 2802 of FIG. 28. The computing device 4000 can communicate with other computing devices.

The computing device 4000 can include a hardware processor 4002, a data storage device 4004, a memory device 4006, a bus 4008, a display 4012, and one or more input/output devices 4014. A processor 4002 can also be implemented as a combination of computing devices, e.g., a combination of a digital signal processor and a microprocessor, a plurality of microprocessors, one or more microprocessors in conjunction with a digital signal processor, or any other such configuration. The processor 4002 can be configured, among other things, to process data, execute instructions to perform one or more functions, such as process one or more physiological signals to obtain one or measurements, as described herein. The data storage device 4004 can include a magnetic disk, optical disk, or flash drive, etc., and is provided and coupled to the bus 4008 for storing information and instructions. The memory 4006 can include one or more memory devices that store data, including without limitation, random access memory (RAM) and read-only memory (ROM). The computing device 4000 may be coupled via the bus 4008 to a display 4012, such as a LCD display or touch screen, for displaying information to a user. The computing device 4000 may be coupled via the bus 4008 to one or more input/output devices 4014. The input device 4014 can include, but is not limited to, a keyboard, mouse, digital pen, microphone, touch screen, gesture recognition system, voice recognition system, imaging device (which may capture eye, hand, head, or body tracking data and/or placement), gamepad, accelerometer, or gyroscope.

### Terminology

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain aspects, the terms “generally parallel” and “substantially parallel” refer to a value, amount, or characteristic that departs from exactly parallel by less than or equal to 10 degrees, 5 degrees, 3 degrees, or 1 degree. As another example, in certain aspects, the terms “generally perpendicular” and “substantially perpendicular” refer to a value, amount, or characteristic that departs from exactly perpendicular by less than or equal to 10 degrees, 5 degrees, 3 degrees, or 1 degree.

Many other variations than those described herein will be apparent from this disclosure. For example, certain acts, events, or functions of any of the algorithms described herein can be performed in a different sequence, can be added, merged, or left out altogether (for example, not all described acts or events are necessary for the practice of the algorithms). Moreover, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

It is to be understood that not necessarily all such advantages can be achieved in accordance with any particular example of the examples disclosed herein. Thus, the examples disclosed herein can be embodied or carried out in a manner that achieves or optimizes one advantage or group of advantages as taught herein without necessarily achieving other advantages as may be taught or suggested herein.

The various illustrative logical blocks, modules, and algorithm steps described in connection with the examples disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the examples disclosed herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can include electrical circuitry or digital logic circuitry configured to process computer-executable instructions. In another example, a processor can include an FPGA or other programmable device that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. A computing environment can include any type of computer system, including, but not limited to, a computer system based on a microprocessor, a mainframe computer, a digital signal processor, a portable computing device, a device controller, or a computational engine within an appliance, to name a few.

The steps of a method, process, or algorithm described in connection with the examples disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC.

The apparatuses and methods described herein may be implemented by one or more computer programs executed by one or more processors. The computer programs include processor-executable instructions that are stored on a non-transitory tangible computer readable medium. The computer programs may also include stored data. Non-limiting examples of the non-transitory tangible computer readable medium are nonvolatile memory, magnetic storage, and optical storage.

The term “substantially” when used in conjunction with the term “real-time” forms a phrase that will be readily understood by a person of ordinary skill in the art. For example, it is readily understood that such language will include speeds in which no or little delay occurs.

Conditional language used herein, such as, among others, “can,” “might,” “may,” “for example,” and the like, unless specifically stated otherwise, or otherwise understood within the context as used, is generally intended to convey that certain examples include, while other examples do not include, certain features, elements and/or states. Thus, such conditional language is not generally intended to imply that features, elements and/or states are in any way required for one or more examples or that one or more examples necessarily include logic for deciding, with or without author input or prompting, whether these features, elements and/or states are included or are to be performed in any particular example. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Disjunctive language such as the phrase “at least one of X, Y, or Z,” unless specifically stated otherwise, is otherwise understood with the context as used in general to present that an item, term, etc., may be either X, Y, or Z, or any combination thereof (for example, X, Y, and/or Z). Thus, such disjunctive language is not generally intended to, and should not, imply that certain examples require at least one of X, at least one of Y, or at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to various examples, it will be understood that various omissions, substitutions, and changes in the form and details of the devices or algorithms illustrated can be made without departing from the spirit of the disclosure. As will be recognized, the inventions described herein can be embodied within a form that does not provide all of the features and benefits set forth herein, as some features can be used or practiced separately from others.

Additionally, all publications, patents, and patent applications mentioned in this specification are herein incorporated by reference to the same extent as if each individual publication, patent, or patent application was specifically and individually indicated to be incorporated by reference.

1. (canceled)

2. A wearable health monitoring device configured to be secured to a wearer, comprising:
a housing including a front side and a back side, the back side configured to face tissue of the wearer when the device is worn; and
a frame positioned by the back side of the housing, said frame defining:
a first emitter chamber housing a first plurality of optical emitters;
a second emitter chamber housing a second plurality of optical emitters, wherein each emitter of the second plurality of optical emitters is configured to emit optical radiation having a same wavelength as optical radiation emitted from a corresponding emitter of the first plurality of optical emitters;
a plurality of detector chambers housing a plurality of optical detectors configured to detect optical radiation emitted from the first and second plurality of optical emitters, wherein the frame is configured to reduce light from the first and second plurality of optical emitters from reaching the plurality of optical detectors without being attenuated by the tissue of the wearer.

3. The wearable health monitoring device of claim 2, wherein the first and second plurality of optical emitters are configured to emit infrared optical radiation and green optical radiation.

4. The wearable health monitoring device of claim 2, wherein the first plurality of optical emitters comprises:
a first optical emitter configured to emit optical radiation having a first wavelength between 620 nm and 660 nm;
a second optical emitter configured to emit optical radiation having a second wavelength between 650 nm and 670 nm; and
a third optical emitter configured to emit optical radiation having a third wavelength between 900 nm and 910 nm.

5. The wearable health monitoring device of claim 4, wherein the second plurality of optical emitters is configured to emit optical radiation having the first, second, and third wavelengths.

6. The wearable health monitoring device of claim 4, wherein the first plurality of optical emitters comprises:
a fourth optical emitter configured to emit optical radiation having a fourth optical wavelength of about 970 nm.

7. The wearable health monitoring device of claim 6, wherein the first plurality of optical emitters comprises:
a fifth optical emitter configured to emit optical radiation having a fifth optical wavelength between 525 nm and 650 nm.

8. The wearable health monitoring device of claim 7, wherein the second plurality of optical emitters is configured to emit optical radiation having the first, second, third, fourth, and fifth wavelengths.

9. The wearable health monitoring device of claim 2, wherein the frame comprises a light barrier configured to separate the first emitter chamber from the second emitter chamber, wherein a width of the light barrier is less than 1.40 mm.

10. The wearable health monitoring device of claim 2, wherein the plurality of detector chambers surround the first and second emitter chambers in an annular formation.

11. The wearable health monitoring device of claim 2, wherein the plurality of detector chambers comprise six detector chambers.

12. The wearable health monitoring device of claim 2,
wherein the first plurality of optical emitters comprises:
a first optical emitter configured to emit optical radiation having a first wavelength; and
a second optical emitter configured to emit optical radiation having a second wavelength;
wherein the second plurality of optical emitters comprises:
a third optical emitter configured to emit optical radiation having the first wavelength; and
a fourth optical emitter configured to emit optical radiation having the second wavelength;
wherein the first and third optical emitters are separated from a first optical detector of the plurality of optical detectors by a first distance;
wherein the second and fourth optical emitters are separated from the first optical detector by a second distance.

13. The wearable health monitoring device of claim 12, wherein the first and second optical emitters are positioned at mirror images from each other across a centerline of the frame that bisects the first optical detector.

14. A wearable health monitoring device configured to be secured to a wearer, comprising:
a housing including a front side and a back side, the back side configured to face tissue of the wearer when the device is worn; and
a frame positioned by the back side of the housing, said frame defining:
a first emitter chamber housing a first plurality of optical emitters, wherein the first plurality of optical emitters is configured to emit at least infrared optical radiation and green optical radiation;
a second emitter chamber housing a second plurality of optical emitters, wherein the second plurality of optical emitters is configured to emit at least infrared optical radiation and green optical radiation;
a plurality of detector chambers housing a plurality of optical detectors configured to detect optical radiation emitted from the first and second plurality of optical emitters, wherein the frame is configured to reduce light from the first and second plurality of optical emitters from reaching the plurality of optical detectors without being attenuated by the tissue of the wearer.

15. The wearable health monitoring device of claim 14, wherein the first plurality of optical emitters comprises:
a first optical emitter configured to emit optical radiation having a first wavelength between 620 nm and 660 nm;
a second optical emitter configured to emit optical radiation having a second wavelength between 650 nm and 670 nm; and
a third optical emitter configured to emit optical radiation having a third wavelength between 900 nm and 910 nm.

16. The wearable health monitoring device of claim 15, wherein the second plurality of optical emitters is configured to emit optical radiation having the first, second, and third wavelengths.

17. The wearable health monitoring device of claim 15, wherein the first plurality of optical emitters comprises:
a fourth optical emitter configured to emit optical radiation having a fourth optical wavelength of about 970 nm; and
a fifth optical emitter configured to emit optical radiation having a fifth optical wavelength between 525 nm and 650 nm.

18. The wearable health monitoring device of claim 2,
wherein the first plurality of optical emitters comprises:
a first optical emitter configured to emit optical radiation having a first wavelength; and
a second optical emitter configured to emit optical radiation having a second wavelength;
wherein the second plurality of optical emitters comprises:
a third optical emitter configured to emit optical radiation having the first wavelength; and
a fourth optical emitter configured to emit optical radiation having the second wavelength;
wherein the first and third optical emitters are separated from a first optical detector of the plurality of optical detectors by a first distance;
wherein the second and fourth optical emitters are separated from the first optical detector by a second distance.

19. A wearable health monitoring device configured to be secured to a wearer, comprising:
a housing including a front side and a back side, the back side configured to face tissue of the wearer when the device is worn; and
a frame positioned by the back side of the housing, said frame defining:
a first emitter chamber housing a first plurality of optical emitters, wherein the first plurality of optical emitters comprises:
a first optical emitter configured to emit optical radiation having a first wavelength; and
a second optical emitter configured to emit optical radiation having a second wavelength;
a second emitter chamber housing a second plurality of optical emitters, wherein the second plurality of optical emitters comprises:
a third optical emitter configured to emit optical radiation having the first wavelength, wherein the first and third optical emitters are each separated from a first optical detector of the plurality of optical detectors by a first distance; and
a fourth optical emitter configured to emit optical radiation having the second wavelength, wherein the second and fourth optical emitters are each separated from the first optical detector by a second distance; and
a plurality of detector chambers housing a plurality of optical detectors configured to detect optical radiation emitted from the first and second plurality of optical emitters, wherein the frame is configured to reduce light from the first and second plurality of optical emitters from reaching the plurality of optical detectors without being attenuated by the tissue of the wearer.

20. The wearable health monitoring device of claim 19, wherein the first and second optical emitters are positioned at mirror images from each other across a centerline of the frame that bisects the first optical detector.

21. The wearable health monitoring device of claim 19, wherein the first plurality of optical emitters comprises:
a first optical emitter configured to emit optical radiation having a first wavelength between 620 nm and 660 nm;
a second optical emitter configured to emit optical radiation having a second wavelength between 650 nm and 670 nm; and
a third optical emitter configured to emit optical radiation having a third wavelength between 900 nm and 910 nm.
